SEQUENCE LISTING (104 sequences)

SEQ ID NO: 1 - protein, 10 aa, synthetic construct
ACSWGIEQRC

SEQ ID NO: 2 - protein, 13 aa, synthetic construct
AGSACSWGIEQRC

SEQ ID NO: 3 - protein, 19 aa, synthetic construct
ACSWGIEQRCGGGENLYFQ

SEQ ID NO: 4 - protein, 22 aa, synthetic construct
AGSACSWGIEQRCGGGENLYFQ

SEQ ID NO: 5 - protein, 10 aa, synthetic construct
  SITE 1: note = Acetylated residue
  SITE 10: note = Amidated residue
CAAAKAAACW

SEQ ID NO: 6 - protein, 6 aa, synthetic construct
  SITE 1: note = Acetylated residue
  SITE 6: note = Amidated residue
CAKACW

SEQ ID NO: 7 - protein, 6 aa, synthetic construct
  SITE 1: note = Acetylated residue
  SITE 6: note = Amidated residue
CYKSCW

SEQ ID NO: 8 - protein, 12 aa, synthetic construct
  SITE 1: note = Acetylated residue
  SITE 12: note = Amidated residue
CAAAAKAAAACW

SEQ ID NO: 9 - protein, 10 aa, synthetic construct
  SITE 1: note = Acetylated residue
  SITE 10: note = Amidated residue
CAEAKAEACW

SEQ ID NO: 10 - protein, 10 aa, synthetic construct
  SITE 1: note = Acetylated residue
  SITE 10: note = Amidated residue
CAHAKAHACW

SEQ ID NO: 11 - protein, 10 aa, synthetic construct
  SITE 1: note = Acetylated residue
  SITE 10: note = Amidated residue
CAKAHQAACW

SEQ ID NO: 12 - protein, 4 aa, synthetic construct
  SITE 1: note = Acetylated residue
  SITE 4: note = Amidated residue
CKCW

SEQ ID NO: 13 - protein, 7 aa, synthetic construct
  SITE 1: note = Acetylated residue
  SITE 7: note = Amidated residue
KACEFCW

SEQ ID NO: 14 - protein, 12 aa, synthetic construct
  SITE 12: note = Amidated residue
GLIGCPFPASWC

SEQ ID NO: 15 - protein, 12 aa, synthetic construct
  SITE 12: note = Amidated residue
ALIGCPFPASWC

SEQ ID NO: 16 - protein, 9 aa, synthetic construct
  SITE 9: note = Amidated residue
ACPFPASWC

SEQ ID NO: 17 - protein, 10 aa, synthetic construct
  SITE 10: note = Amidated residue
ACLIPTWGGC

SEQ ID NO: 18 - protein, 9 aa, synthetic construct
  SITE 9: note = Amidated residue
PCPFPASWC

SEQ ID NO: 19 - protein, 12 aa, synthetic construct
  SITE 12: note = Amidated residue
ACFMQEPLYICG

SEQ ID NO: 20 - protein, 16 aa, synthetic construct
  SITE 16: note = Amidated residue
ALIGCESAYKNTAQCW

SEQ ID NO: 21 - protein, 10 aa, synthetic construct
  SITE 1: note = Acetylated residue
  SITE 10: note = Amidated residue
CAAARAAACW

SEQ ID NO: 22 - protein, 12 aa, synthetic construct
  SITE 12: note = Amidated residue
ALIGCPFPARWC

SEQ ID NO: 23 - protein, 11 aa, synthetic construct
CAAAAKAAAAC

SEQ ID NO: 24 - DNA, 30 bp, synthetic construct
  source 1..30: mol_type = other DNA
ccgggtaccagtacattagaggttagatca

SEQ ID NO: 25 - DNA, 29 bp, synthetic construct
  source 1..29: mol_type = other DNA
cgggcggccgctaaattcacttcaatttt

SEQ ID NO: 26 - DNA, 25 bp, synthetic construct
  source 1..25: mol_type = other DNA
ccatggagagcaccaccaccaccac

SEQ ID NO: 27 - DNA, 27 bp, synthetic construct
  source 1..27: mol_type = other DNA
gctgcggcgagtgcggccgctaaattc

SEQ ID NO: 28 - DNA, 22 bp, synthetic construct
  source 1..22: mol_type = other DNA
tgagatccggctgctaacaaag

SEQ ID NO: 29 - DNA, 18 bp, synthetic construct
  source 1..18: mol_type = other DNA
gccaccaccccaacatgc

SEQ ID NO: 30 - DNA, 28 bp, synthetic construct
  source 1..28: mol_type = other DNA
caccaccacggcctgaacgatatttttg

SEQ ID NO: 31 - DNA, 27 bp, synthetic construct
  source 1..27: mol_type = other DNA
atgatgatgcatcggtaattgttcctc

SEQ ID NO: 32 - protein, 248 aa, synthetic construct
MHHHHHHGLNDIFEAQKIEWHEGTSTLEVRSQATQDLSEYYKGRGFELTNVTGYKYGNKVTFIDNSQQIDVTLTGNEKLTVKDDDEVSNVDVFVVREGSDKSAITTSIGGITKTNGTQHKDTVQNVNLSVSKSTGQHTTSVTSEYYSIYKEEISLKELDFKLRKHLIDKHDLYKTEPKDSKIRITMKNGGYYTFELNKKLQPHRMGDTIDSRNIEKIEVNLAAAENLYFQGGGCAAAAKAAAACWGGG

SEQ ID NO: 33 - protein, 6 aa, synthetic construct
ENLYFQ

SEQ ID NO: 34 - DNA, 24 bp, synthetic construct
  source 1..24: mol_type = other DNA
taccagcagagtgagaatagaaag

SEQ ID NO: 35 - DNA, 23 bp, synthetic construct
  source 1..23: mol_type = other DNA
gtgcttgtagttggggtattgag

SEQ ID NO: 36 - protein, 10 aa, synthetic construct
  SITE 1: note = Acetylated residue
  SITE 10: note = Amidated residue
CAAAKAAACW

SEQ ID NO: 37 - protein, 17 aa, synthetic construct
GGCAAAAKAAAACWGGG

SEQ ID NO: 38 - protein, 7 aa, synthetic construct
ENLYFQG

SEQ ID NO: 39 - protein, 7 aa, synthetic construct
ENLYFQS

SEQ ID NO: 40 - protein, 13 aa, synthetic construct
AIDSCRYNHGWQC

SEQ ID NO: 41 - protein, 13 aa, synthetic construct
AISECNHIAEWMC

SEQ ID NO: 42 - protein, 10 aa, synthetic construct
  SITE 1: note = Acetylated residue
  SITE 10: note = Amidated residue
  VARIANT 1..5: note = Residues linked via 3,5-bischlorooxime-benzoic acid tetrafluorophenol ester or 3,5-bischlorooxime-benzoic acid N-succinimidyl ester
  VARIANT 5..9: note = Residues linked via 3,5-bischlorooxime-benzoic acid tetrafluorophenol ester or 3,5-bischlorooxime-benzoic acid N-succinimidyl ester
  VARIANT 1..9: note = Residues linked via 3,5-bischlorooxime-benzoic acid tetrafluorophenol ester or 3,5-bischlorooxime-benzoic acid N-succinimidyl ester
CAAAKAAACW

SEQ ID NO: 43 - protein, 10 aa, synthetic construct
  SITE 1: note = Acetylated residue
  SITE 10: note = Amidated residue
  SITE 1..5: note = Residues linked via 1,3-dichloroacetone-N-succinimidyl ester
  SITE 5..9: note = Residues linked via 1,3-dichloroacetone-N-succinimidyl ester
  SITE 1..9: note = Residues linked via 1,3-dichloroacetone-N-succinimidyl ester
CAAAKAAACW

SEQ ID NO: 44 - protein, 6 aa, synthetic construct
  SITE 1: note = Acetylated residue
  SITE 6: note = Amidated residue
KSCAKW

SEQ ID NO: 45 - protein, 6 aa, synthetic construct
  SITE 1: note = Acetylated residue
  SITE 6: note = Amidated residue
  SITE 1..3: note = Residues linked via 1-chlorooxime-3,5-bisbenzoic acid-N-succinimidyl ester
  SITE 3..5: note = Residues linked via 1-chlorooxime-3,5-bisbenzoic acid-N-succinimidyl ester
  SITE 1..5: note = Residues linked via 1-chlorooxime-3,5-bisbenzoic acid-N-succinimidyl ester
KSCAKW

SEQ ID NO: 46 - protein, 10 aa, synthetic construct
  SITE 1: note = Acetylated residue
  SITE 10: note = Amidated residue
  SITE 1..9: note = Residues linked via 3,5-bischolorooxime-benzoic acid-difluorophenol ester
CAAAKAAACW

SEQ ID NO: 47 - protein, 6 aa, synthetic construct
  SITE 1: note = Acetylated residue
  SITE 6: note = Amidated residue
  SITE 1..3: note = Residues linked via 3,5-bischlorooxime-benzoic acid N-succinimidyl ester
  SITE 3..5: note = Residues linked via 3,5-bischlorooxime-benzoic acid N-succinimidyl ester
  SITE 1..5: note = Residues linked via 3,5-bischlorooxime-benzoic acid N-succinimidyl ester
CAKACW

SEQ ID NO: 48 - protein, 6 aa, synthetic construct
  SITE 1: note = Acetylated residue
  SITE 6: note = Amidated residue
  SITE 1..3: note = Residues linked via 3,5-bischlorooxime-benzoic acid tetrafluorophenol ester
  SITE 3..5: note = Residues linked via 3,5-bischlorooxime-benzoic acid tetrafluorophenol ester
  SITE 1..5: note = Residues linked via 3,5-bischlorooxime-benzoic acid tetrafluorophenol ester
CYKSCW

SEQ ID NO: 49 - protein, 12 aa, synthetic construct
  SITE 1: note = Acetylated residue
  SITE 12: note = Amidated residue
  SITE 1..6: note = Residues linked via 3,5-bischlorooxime-benzoic acid N-succinimidyl ester
  SITE 6..11: note = Residues linked via 3,5-bischlorooxime-benzoic acid N-succinimidyl ester
  SITE 1..11: note = Residues linked via 3,5-bischlorooxime-benzoic acid N-succinimidyl ester
CAAAAKAAAACW

SEQ ID NO: 50 - protein, 10 aa, synthetic construct
  SITE 1: note = Acetylated residue
  SITE 10: note = Amidated residue
  SITE 1..5: note = Residues linked via 3,5-bischlorooxime-benzoic acid tetrafluorophenol ester
  SITE 5..9: note = Residues linked via 3,5-bischlorooxime-benzoic acid tetrafluorophenol ester
  SITE 1..9: note = Residues linked via 3,5-bischlorooxime-benzoic acid tetrafluorophenol ester
CAEAKAEACW

SEQ ID NO: 51 - protein, 10 aa, synthetic construct
  SITE 1: note = Acetylated residue
  SITE 10: note = Amidated residue
  SITE 1..5: note = Residues linked via 3,5-bischlorooxime-benzoic acid N-succinimidyl ester
  SITE 5..9: note = Residues linked via 3,5-bischlorooxime-benzoic acid N-succinimidyl ester
  SITE 1..9: note = Residues linked via 3,5-bischlorooxime-benzoic acid N-succinimidyl ester
CAHAKAHACW

SEQ ID NO: 52 - protein, 10 aa, synthetic construct
  SITE 1: note = Acetylated residue
  SITE 10: note = Amidated residue
  SITE 1..3: note = Residues linked via 3,5-bischlorooxime-benzoic acid tetrafluorophenol ester
  SITE 3..9: note = Residues linked via 3,5-bischlorooxime-benzoic acid tetrafluorophenol ester
  SITE 1..9: note = Residues linked via 3,5-bischlorooxime-benzoic acid tetrafluorophenol ester
CAKAHQAACW

SEQ ID NO: 53 - protein, 4 aa, synthetic construct
  SITE 1: note = Acetylated residue
  SITE 4: note = Amidated residue
  SITE 1..2: note = Residues linked via 3,5-bischlorooxime-benzoic acid N-succinimidyl ester
  SITE 2..3: note = Residues linked via 3,5-bischlorooxime-benzoic acid N-succinimidyl ester
  SITE 1..3: note = Residues linked via 3,5-bischlorooxime-benzoic acid N-succinimidyl ester
CKCW

SEQ ID NO: 54 - protein, 7 aa, synthetic construct
  SITE 1: note = Acetylated residue
  SITE 7: note = Amidated residue
  SITE 1..3: note = Residues linked via 3,5-bischlorooxime-benzoic acid tetrafluorophenol ester
  SITE 3..6: note = Residues linked via 3,5-bischlorooxime-benzoic acid tetrafluorophenol ester
  SITE 1..6: note = Residues linked via 3,5-bischlorooxime-benzoic acid tetrafluorophenol ester
KACEFCW

SEQ ID NO: 55 - protein, 12 aa, synthetic construct
  SITE 12: note = Amidated residue
  SITE 1..5: note = Residues linked via 3,5-bischlorooxime-benzoic acid N-succinimidyl ester
  SITE 5..12: note = Residues linked via 3,5-bischlorooxime-benzoic acid N-succinimidyl ester
  SITE 1..12: note = Residues linked via 3,5-bischlorooxime-benzoic acid N-succinimidyl ester
GLIGCPFPASWC

SEQ ID NO: 56 - protein, 12 aa, synthetic construct
  SITE 12: note = Amidated residue
  SITE 1..5: note = Residues linked via 3,5-bischlorooxime-benzoic acid N-succinimidyl ester
  SITE 5..12: note = Residues linked via 3,5-bischlorooxime-benzoic acid N-succinimidyl ester
  SITE 1..12: note = Residues linked via 3,5-bischlorooxime-benzoic acid N-succinimidyl ester
ALIGCPFPASWC

SEQ ID NO: 57 - protein, 9 aa, synthetic construct
  SITE 9: note = Amidated residue
  SITE 1..2: note = Residues linked via 3,5-bischlorooxime-benzoic acid N-succinimidyl ester
  SITE 2..9: note = Residues linked via 3,5-bischlorooxime-benzoic acid N-succinimidyl ester
  SITE 1..9: note = Residues linked via 3,5-bischlorooxime-benzoic acid N-succinimidyl ester
ACPFPASWC

SEQ ID NO: 58 - protein, 10 aa, synthetic construct
  SITE 10: note = Amidated residue
  SITE 1..2: note = Residues linked via 3,5-bischlorooxime-benzoic acid tetrafluorophenol ester
  SITE 2..10: note = Residues linked via 3,5-bischlorooxime-benzoic acid tetrafluorophenol ester
  SITE 1..10: note = Residues linked via 3,5-bischlorooxime-benzoic acid tetrafluorophenol ester
ACLIPTWGGC

SEQ ID NO: 59 - protein, 9 aa, synthetic construct
  SITE 9: note = Amidated residue
  SITE 1..2: note = Residues linked via 3,5-bischlorooxime-benzoic acid tetrafluorophenol ester
  SITE 2..9: note = Residues linked via 3,5-bischlorooxime-benzoic acid tetrafluorophenol ester
  SITE 1..9: note = Residues linked via 3,5-bischlorooxime-benzoic acid tetrafluorophenol ester
PCPFPASWC

SEQ ID NO: 60 - protein, 12 aa, synthetic construct
  SITE 12: note = Amidated residue
  SITE 1..2: note = Residues linked via 3,5-bischlorooxime-benzoic acid tetrafluorophenol ester
  SITE 2..11: note = Residues linked via 3,5-bischlorooxime-benzoic acid tetrafluorophenol ester
  SITE 1..11: note = Residues linked via 3,5-bischlorooxime-benzoic acid tetrafluorophenol ester
ACFMQEPLYICG

SEQ ID NO: 61 - protein, 16 aa, synthetic construct
  SITE 16: note = Amidated residue
  SITE 1..5: note = Residues linked via 3,5-bischlorooxime-benzoic acid N-succinimidyl ester
  SITE 5..15: note = Residues linked via 3,5-bischlorooxime-benzoic acid N-succinimidyl ester
  SITE 1..15: note = Residues linked via 3,5-bischlorooxime-benzoic acid N-succinimidyl ester
ALIGCESAYKNTAQCW

SEQ ID NO: 62 - protein, 12 aa, synthetic construct
  SITE 12: note = Amidated residue
  SITE 1..5: note = Residues linked via 3,5-bischlorooxime-benzoic acid N-succinimidyl ester
  SITE 5..12: note = Residues linked via 3,5-bischlorooxime-benzoic acid N-succinimidyl ester
  SITE 1..12: note = Residues linked via 3,5-bischlorooxime-benzoic acid N-succinimidyl ester
ALIGCPFPARWC

SEQ ID NO: 63 - protein, 9 aa, synthetic construct
  SITE 9: note = Amidated residue
  SITE 2..9: note = Intrachain disulfide bond
PCPFPASWC

SEQ ID NO: 64 - protein, 24 aa, synthetic construct
ENLYFQGGGCAAAAKAAAACWGGG

SEQ ID NO: 65 - protein, 24 aa, synthetic construct
  SITE 10..15: note = Residues linked via 3,5-bischlorooxime-benzoic acid N-succinimidyl ester
  SITE 15..20: note = Residues linked via 3,5-bischlorooxime-benzoic acid N-succinimidyl ester
  SITE 10..20: note = Residues linked via 3,5-bischlorooxime-benzoic acid N-succinimidyl ester
ENLYFQGGGCAAAAKAAAACWGGG

SEQ ID NO: 66 - protein, 23 aa, synthetic construct
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 20: note = Any amino acid
  VARIANT 21: note = Any amino acid
  VARIANT 22: note = Any amino acid
  SITE 11..19: note = Residues linked via 3,5-bischlorooxime-benzoic acid N-succinimidyl ester
  SITE 19..23: note = Residues linked via 3,5-bischlorooxime-benzoic acid N-succinimidyl ester
  SITE 11..23: note = Residues linked via 3,5-bischlorooxime-benzoic acid N-succinimidyl ester
SQFYLNEGGGCXXXXXXXCXXXA

SEQ ID NO: 67 - protein, 22 aa, synthetic construct
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 19: note = Any amino acid
  VARIANT 20: note = Any amino acid
  VARIANT 21: note = Any amino acid
  SITE 10..18: note = Residues linked via 3,5-bischlorooxime-benzoic acid N-succinimidyl ester
  SITE 18..22: note = Residues linked via 3,5-bischlorooxime-benzoic acid N-succinimidyl ester
  SITE 10..22: note = Residues linked via 3,5-bischlorooxime-benzoic acid N-succinimidyl ester
QFYLNEGGGCXXXXXXXCXXXA

SEQ ID NO: 68 - protein, 10 aa, synthetic construct
  SITE 1: note = Acetylated residue
  SITE 10: note = Amidated residue
  VARIANT 1..5: note = Residues linked via 3,5-bischlorooxime-benzoic acid tetrafluorophenol ester, 3,5-bischlorooxime-benzoic acid N-succinimidyl ester, 3,5-bischolorooxime-benzoic acid-difluorophenol ester, bis-bromomethyl-benzoic acid N-succinimidyl ester, or 1,3-dichloroacetone-N-succinimidyl ester
  VARIANT 5..9: note = Residues linked via 3,5-bischlorooxime-benzoic acid tetrafluorophenol ester, 3,5-bischlorooxime-benzoic acid N-succinimidyl ester, 3,5-bischolorooxime-benzoic acid-difluorophenol ester, bis-bromomethyl-benzoic acid N-succinimidyl ester, or 1,3-dichloroacetone-N-succinimidyl ester
  VARIANT 1..9: note = Residues linked via 3,5-bischlorooxime-benzoic acid tetrafluorophenol ester, 3,5-bischlorooxime-benzoic acid N-succinimidyl ester, 3,5-bischolorooxime-benzoic acid-difluorophenol ester, bis-bromomethyl-benzoic acid N-succinimidyl ester, or 1,3-dichloroacetone-N-succinimidyl ester
CAAAKAAACW

SEQ ID NO: 69 - protein, 10 aa, synthetic construct
  SITE 1: note = Acetylated residue
CAAARAAACW

SEQ ID NO: 70 - protein, 5 aa, synthetic construct
  SITE 1: note = Acetylated residue
CAAAR

SEQ ID NO: 71 - protein, 4 aa, synthetic construct
CKCW

SEQ ID NO: 72 - protein, 5 aa, synthetic construct
GLIGC

SEQ ID NO: 73 - protein, 4 aa, synthetic construct
TWGG

SEQ ID NO: 74 - protein, 9 aa, synthetic construct
PCPFPASWC

SEQ ID NO: 75 - protein, 10 aa, synthetic construct
  SITE 1: note = Acetylated residue
CAAAKAAACW

SEQ ID NO: 76 - protein, 5 aa, synthetic construct
  SITE 1: note = Acetylated residue
CAAAK

SEQ ID NO: 77 - DNA, 92 bp, synthetic construct
  source 1..92: mol_type = other DNA
cgggcggccgcagaaaatctttattttcaaggtggtggttgtgcagcagcagcaaaggcagcagcagcatgttggggtggtggccatggcgg

SEQ ID NO: 78 - protein, 29 aa, synthetic construct
AAAENLYFQGGGCAAAAKAAAACWGGGHG

SEQ ID NO: 79 - protein, 12 aa, synthetic construct
CAAAAKAAAACW

SEQ ID NO: 80 - protein, 18 aa, synthetic construct
  SITE 4..9: note = Residues linked via 3,5-bischlorooxime-benzoic acid N-succinimidyl ester
  SITE 9..14: note = Residues linked via 3,5-bischlorooxime-benzoic acid N-succinimidyl ester
  SITE 4..14: note = Residues linked via 3,5-bischlorooxime-benzoic acid N-succinimidyl ester
GGGCAAAAKAAAACWGGG

SEQ ID NO: 81 - DNA, 140 bp, synthetic construct
  source 1..140: mol_type = other DNA
gctaaacaactttcaacagtttcggccgacgactgaaaatacaggttttcacctccaccacaacgctgctcaataccccaactacaagcagagtgagaatagaaaggtaccactaaaggaattgcgaataataatttttt

SEQ ID NO: 82 - protein, 46 aa, synthetic construct
LCSEVTEASSQFYLNEGGGCRQEIGWSCASHSYFPVVLPIAFLLKK

SEQ ID NO: 83 - DNA, 148 bp, synthetic construct
  source 1..148: mol_type = other DNA
ctaaacaactttcaacagtttcggccgacgactgaaaatacaggttttcacctccaccacaacgctgctcaataccccaactacaagcactaccagcagagtgagaatagaaaggtaccactaaaggaattgcgaataataatttttt

SEQ ID NO: 84 - protein, 49 aa, synthetic construct
LCSEVTEASSQFYLNEGGGCRQEIGWSCASGASHSYFPVVLPIAFLLKK

SEQ ID NO: 85 - protein, 22 aa, synthetic construct
  SITE 5..13: note = Intrachain disulfide bond
AGSACSWGIEQRCGGGENLYFQ

SEQ ID NO: 86 - protein, 22 aa, synthetic construct
  VARIANT 5..13: note = Residues linked via 3,5-bischlorooxime-benzoic acid tetrafluorophenol ester or 3,5-bischlorooxime-benzoic acid N-succinimidyl ester
AGSACSWGIEQRCGGGENLYFQ

SEQ ID NO: 87 - protein, 10 aa, synthetic construct
  SITE 1: note = Acetylated residue
  SITE 10: note = Amidated residue
  SITE 1..9: note = Residues linked via 3,5-bischolorooxime-benzoic acid-difluorophenol ester
CAAAKAAACW

SEQ ID NO: 88 - protein, 10 aa, synthetic construct
  SITE 1: note = Acetylated residue
  SITE 10: note = Amidated residue
  SITE 1..5: note = Residues linked via bis-bromomethyl-benzoic acid N-succinimidyl ester
  SITE 5..9: note = Residues linked via bis-bromomethyl-benzoic acid N-succinimidyl ester
  SITE 1..9: note = Residues linked via bis-bromomethyl-benzoic acid N-succinimidyl ester
CAAAKAAACW

SEQ ID NO: 89 - protein, 10 aa, synthetic construct
  SITE 1: note = Acetylated residue
  SITE 10: note = Amidated residue
  SITE 1..5: note = Residues linked via bis-bromomethyl-benzoic acid N-succinimidyl ester
CAAAKAAACW

SEQ ID NO: 90 - protein, 10 aa, synthetic construct
  SITE 1: note = Acetylated residue
  SITE 10: note = Amidated residue
  SITE 5..9: note = Residues linked via bis-bromomethyl-benzoic acid N-succinimidyl ester
CAAAKAAACW

SEQ ID NO: 91 - protein, 10 aa, synthetic construct
  SITE 1: note = Acetylated residue
  SITE 10: note = Amidated residue
  SITE 1..9: note = Residues linked via bis-bromomethyl-benzoic acid N-succinimidyl ester
CAAAKAAACW

SEQ ID NO: 92 - protein, 10 aa, synthetic construct
  SITE 1: note = Acetylated residue
  SITE 10: note = Amidated residue
  SITE 1..5: note = Residues linked via 1,3-dichloroacetone-N-succinimidyl ester
CAAAKAAACW

SEQ ID NO: 93 - protein, 10 aa, synthetic construct
  SITE 1: note = Acetylated residue
  SITE 10: note = Amidated residue
  SITE 5..9: note = Residues linked via 1,3-dichloroacetone-N-succinimidyl ester
CAAAKAAACW

SEQ ID NO: 94 - protein, 10 aa, synthetic construct
  SITE 1: note = Acetylated residue
  SITE 10: note = Amidated residue
  SITE 1..9: note = Residues linked via 1,3-dichloroacetone-N-succinimidyl ester
CAAAKAAACW

SEQ ID NO: 95 - protein, 10 aa, synthetic construct
  SITE 1: note = Acetylated residue
  SITE 10: note = Amidated residue
  SITE 1..9: note = Residues linked via 1,3-bischlorooxime-benzene
CAAAKAAACW

SEQ ID NO: 96 - protein, 10 aa, synthetic construct
  SITE 1: note = Acetylated residue
  SITE 10: note = Amidated residue
  SITE 1..9: note = Intrachain disulfide bond
CAAAKAAACW

SEQ ID NO: 97 - protein, 10 aa, synthetic construct
  SITE 1: note = Acetylated residue
  SITE 10: note = Amidated residue
  SITE 1..9: note = Residues linked via 3,5-bischlorooxime-benzoic acid N-succinimidyl ester
CAAARAAACW

SEQ ID NO: 98 - protein, 4 aa, synthetic construct
  SITE 1: note = Acetylated residue
  SITE 4: note = Amidated residue
  SITE 1..3: note = Residues linked via 3,5-bischlorooxime-benzoic acid tetrafluorophenol ester
CKCW

SEQ ID NO: 99 - protein, 4 aa, synthetic construct
  SITE 1: note = Acetylated residue
  SITE 4: note = Amidated residue
  SITE 1..3: note = Residues linked via 3,5-bischlorooxime-benzoic acid tetrafluorophenol ester
CKCW

SEQ ID NO: 100 - protein, 16 aa, synthetic construct
  SITE 16: note = Amidated residue
  SITE 5..10: note = Residues linked via 3,5-bischlorooxime-benzoic acid N-succinimidyl ester
  SITE 10..15: note = Residues linked via 3,5-bischlorooxime-benzoic acid N-succinimidyl ester
  SITE 5..15: note = Residues linked via 3,5-bischlorooxime-benzoic acid N-succinimidyl ester
ALIGCESAYKNTAQCW

SEQ ID NO: 101 - protein, 10 aa, synthetic construct
  SITE 1: note = Acetylated residue
  SITE 5: note = Chlorooxime-free tetrafluorophenol crosslinker modified residue
  SITE 10: note = Amidated residue
  SITE 1..9: note = Intrachain disulfide bond
CAAAKAAACW

SEQ ID NO: 102 - protein, 10 aa, synthetic construct
  SITE 10: note = Amidated residue
  SITE 2..10: note = Residues linked via 3,5-bischlorooxime-benzoic acid tetrafluorophenol ester
ACLIPTWGGC

SEQ ID NO: 103 - protein, 22 aa, synthetic construct
  VARIANT 1..5: note = Residues linked via 3,5-bischlorooxime-benzoic acid tetrafluorophenol ester or 3,5-bischlorooxime-benzoic acid N-succinimidyl ester
  VARIANT 5..13: note = Residues linked via 3,5-bischlorooxime-benzoic acid tetrafluorophenol ester or 3,5-bischlorooxime-benzoic acid N-succinimidyl ester
  VARIANT 1..13: note = Residues linked via 3,5-bischlorooxime-benzoic acid tetrafluorophenol ester or 3,5-bischlorooxime-benzoic acid N-succinimidyl ester
AGSACSWGIEQRCGGGENLYFQ

SEQ ID NO: 104 - protein, 18 aa, synthetic construct
  SITE 4..9: note = Residues linked via 3,5-bischlorooxime-benzoic acid N-succinimidyl ester
  SITE 9..14: note = Residues linked via 3,5-bischlorooxime-benzoic acid N-succinimidyl ester
  SITE 4..14: note = Residues linked via 3,5-bischlorooxime-benzoic acid N-succinimidyl ester
GGGCAAAAKAAAACWGGG

(19) United States
(12) Patent Application Publication
(10) Pub. No.: US 2026/0132547 A1
(43) Pub. Date: May 14, 2026

(54) NOVEL PHAGE LIBRARIES OF BICYCLIC PEPTIDES

(71) Applicant: The Trustees of Boston College, Chestnut Hill, MA (US)

(72) Inventor: Jianmin GAO, Newton, MA (US)

(73) Assignee: The Trustees of Boston College, Chestnut Hill, MA (US)

(21) Appl. No.: 19/392,299

(22) Filed: Nov. 18, 2025

Related U.S. Application Data
(63) Continuation of application No. PCT/US2024/030810, filed on May 23, 2024.

(51) Int. Cl. C40B 40/02; C07K 1/13; C07K 2/00; C40B 30/04; C40B 50/06

(52) U.S. Cl. CPC C40B 40/02; C07K 1/13; C07K 2/00; C40B 30/04; C40B 50/06

(57) ABSTRACT

A cysteine-directed proximity-driven strategy for the construction of bicyclic peptides from simple natural peptide precursors. This linear to bicycle transformation initiates with rapid cysteine labeling, which then triggers proximity-driven amine-selective cyclization. This bicyclization proceeds rapidly under physiologic conditions, yielding bicyclic peptides with a Cys-Lys-Cys, Lys-Cys-Lys or N-terminus-Cys-Cys stapling pattern. Novel phage libraries of bicyclic peptides and method of use thereof for screening therapeutic proteins.

### CROSS-REFERENCE

This application is continuation of a PCT International Application No. PCT/US2024/030810, filed on May 23, 2024, which claims the benefit of U.S. Provisional Application No. 63/504,195, filed May 24, 2024, the entire content of each of which is incorporated herein by reference.

### STATEMENT REGARDING FEDERALLY SPONSORED RESEARCH OR DEVELOPMENT

This invention was made with government support under grant number CHE2204078 awarded by the National Science Foundation. The government has certain rights in the invention.

### SEQUENCE LISTING

The instant application contains a Sequence Listing which has been submitted electronically in XML format and is hereby incorporated by reference in its entirety. Said XML copy, created on Oct. 15, 2025, is named 940203-1150US_SL.xml and is 181,822 bytes in size.

### FIELD AND BACKGROUND

Multicyclic peptides are commonly seen in bioactive natural products, such as the antibiotic of last resort vancomycin and the cytotoxin α-amanitin (FIG. 1A).^{[1]} Compared to linear or simple disulfide-cyclized peptides, chemically multicyclized peptides have greater conformational rigidity as well as metabolic stability.^{[2]} Importantly, sitting in the chemical space between small molecule drugs and biologics, multicyclic peptides are considered an appealing modality for drug discovery.^{[3]} In particular, they have been shown to exhibit high potency for inhibiting protein-protein interactions (PPIs),^{[4]} which are often deemed undruggable by synthetic molecules. However, it remains difficult to discover multicyclic peptides with desired bioactivities, at least in part due to their synthetic challenges, which prohibit facile access to diverse multicyclic peptides for library screening.^{[5]} Peptide libraries can be arguably best prepared through genetically encoded approaches such as phage display or mRNA display.^{[5-6]}

Multicyclization of library peptides demands site-specific transformation of native peptides under physiologic conditions. Although many methods are reported for test-tube cyclization of peptides, most require nonnatural peptide substrates or non-biocompatible conditions (e.g., water-free media).^{[7]} Only a handful of methods allow efficient and site-specific bicyclization of native peptides on phage (FIG. 1B).^{[8]} Although clever in design, these methods are less ideal as they require a heavily engineered cysteine-free phage,^{[9]} harsh oxidation conditions,^{[10]} or produce bicyclic isomers.^{[11-12]} As an alternative to phage display, mRNA display only achieved bicyclic peptide libraries using engineered systems that incorporate unnatural amino acids.^{[13]}

Efficient and site-specific modification of native peptides and proteins is desirable for synthesizing antibody-drug conjugates as well as for constructing chemically modified peptide libraries using genetically encoded platforms such as phage display. In particular, there is much interest in efficient multicyclization of native peptides due to the appeals of multicyclic peptides as therapeutics. However, conventional approaches for multicyclic peptide synthesis require orthogonal protecting groups or non-proteinogenic clickable handles. Therefore, additional methods are still needed to allow facile cyclization of native peptides to access structurally novel libraries.

### SUMMARY

The present disclosure provides a cysteine-directed proximity-driven strategy for bicyclizing native peptides, which enables facile construction of phage libraries of peptide bicycles (FIG. 1C). The reaction initiates with rapid cysteine conjugation to chlorooxime-based crosslinkers, which then set up proximity-driven conjugation with amines to give bicyclic peptides. This strategy is general and robust, enabling rapid Cys-Lys-Cys stapling, Lys-Cys-Lys stapling, as well as N-terminus-Cys-Cys stapling of diverse peptide sequences. In certain embodiments, a panel of backbone and/or sidechain bicyclized peptides were constructed with high efficiency. The biocompatibility and exquisite site selectivity of this novel protocol were further demonstrated by bicyclization of peptides fused to a carrier protein and bacteriophage.

The present disclosure also provides a novel and highly efficient way of constructing bicyclic peptide libraries. Previously known methods for such a purpose involve the use of heavily engineered carrier phage, harsh oxidation conditions, or the risk of generating multiple bicyclic isomers. The method disclosed herein allows one-step modification of wild-type carrier phage to give structurally complex bicyclic peptide libraries, which can be used to identify potent inhibitors for various biological targets.

The present disclosure further provides useful screenings for inhibitors of therapeutically important proteins. Many therapeutically important proteins, particularly those involved in protein-protein interactions, cannot be inhibited by traditional small molecule drugs. Bicyclic peptides offer a privileged scaffold as well as enormous sequence diversity, which make them a rich ground for discovering potent inhibitors. The present disclosure provides methods to build libraries with more structurally complex peptides and screen against various proteins of therapeutic indications.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIGS. 1A-1C. Biocompatible peptide bicyclization. FIG. 1A (Prior art). Multicyclic peptides in biologically active natural products. FIG. 1B (Prior art). Encoded bicyclic peptide libraries. FIG. 1C. Proximity-driven strategy for native peptide bicyclization under biocompatible conditions.

FIGS. 2A-2F. Design and evaluation of protocols for native peptide bicyclization. General reaction conditions unless otherwise noted: linear peptide (10 μM), crosslinker (20 μM) in 10 mM PBS/acetone (v/v 80/20), pH 7.4, incubated at room temperature for 10 minutes. FIG. 2A. Structures of crosslinkers. Chlorooxime, alkyl halides are cysteine-reactive handles. Activated esters are lysine-reactive handles. FIG. 2B. Bicyclization of model peptide P1 with crosslinkers 2a-e through Cys-Lys-Cys stapling. Figure discloses SEQ ID NOS 5, 42, and 88, and 43, respectively, in order of appearance. FIG. 2C. LC traces (280 nm) of P1 bicyclization with 3a-e. FIG. 2D. Bicyclization of model peptide P17 with crosslinkers 3h through Lys-Cys-Lys stapling. Figure discloses SEQ ID NOS 44-45, respectively, in order of appearance. FIG. 2E. LC traces (280 nm) of crude reaction mixtures for P17 bicyclization with 3h. FIG. 2F. Monitor of the monocyclic intermediate. BC: bischlorooxime. TFP: tetrafluoro phenol ester. OSu: N-succinimidyl ester. DFP: difluorophenol ester. CO: chlorooxime. Figure discloses SEQ ID NO: 46.

FIGS. 3A-3D. Substrate scope of peptide bicyclization. General reaction conditions unless otherwise noted: linear peptide (10 μM), crosslinker 3a or 3b (20 μM) in PBS/acetone (v/v 80/20), pH 7.4, incubated at room temperature for 10 minutes. [m,n]: numbers of spacer amino acids between modified residues. FIG. 3A. Peptide bicyclization through endo-Cys-Lys-Cys stapling. Figure discloses SEQ ID NOS 47-53, respectively, in order of appearance. FIG. 3B. Peptide bicyclization through exo-Lys-Cys-Cys stapling. Figure discloses SEQ ID NO: 54. FIG. 3C. Peptide bicyclization through N-terminus-Cys-Cys stapling. Figure discloses SEQ ID NOS 55-62, respectively, in order of appearance. FIG. 3D. Disulfide peptide re-bridging through TCEP reduction/bicyclization. ^{a}4 hours. ^{b}120 hours. ^{c}mixture of products bicyclized at lysine side chain and N-terminus. ^{d}linear peptide (10 μM), crosslinker 3b (30 μM), TCEP (10 μM) in PBS/acetonitrile (v/v 80/20), pH 7.4, incubated at room temperature for 10 minutes. ^{e}yield of 1 mM scale. Figure discloses SEQ ID NOS 63, 18, and 59, respectively, in order of appearance.

FIGS. 4A-4C. Cysteine-directed bicyclization of a fusion protein (PBD: 2RDH). FIG. 4A. SSL11 modification and TEV cleavage. Bicyclization conditions: reduced SSL11 protein 6a (40 μM), crosslinker 3b (80 μM) in PBS/acetone (v/v 92/8), pH 7.4, incubated at room temperature for 15 minutes. TEV cleavage conditions: bicyclic protein 6b (36 μM), TEV protease (0.8 μM) in PBS, pH 7.4, incubated at room temperature overnight. Figure discloses SEQ ID NOS 64-65, and 33, respectively, in order of appearance. FIG. 4B. Deconvoluted mass for SSL11 protein modification. FIG. 4C. peptide fragment 6d obtained by TEV cleavage. Figure discloses SEQ ID NO: 104.

FIGS. 5A-5C. Cysteine-directed bicyclization on M13 bacteriophage. FIG. 5A. The modified phage is cleaved with TEV protease to release the bicyclic peptide fragment, which is then analyzed by LC-MS. Figure discloses SEQ ID NOS 66-67, respectively, in order of appearance. FIG. 5B. The chemical structure of the bicyclic ACX₇C peptide, 7a. The [M+2H]²⁺ extracted masses of the unmodified (purple), bicyclic (pink), and hydrolyzed (blue) peptides in the sample. FIG. 5C. The chemical structure of the bicyclic AX₃CX₇C peptide, 7b. The [M+2H]²⁺ extracted masses of the unmodified (purple), bicyclic (pink), and hydrolyzed (blue) peptides in the sample. *Mass spectrometry artifact. [m,n]: numbers of spacer amino acid. EIC: extracted ion chromatogram. ACX₇C: ACSWGIEQRC (SEQ ID NO: 1). AX₃CX₇C: AGSACSWGIEQRC (SEQ ID NO: 2).

FIGS. 6a-6c illustrate a reaction scheme of peptide bicyclization (FIG. 6a; SEQ ID NOS 5 and 68, respectively, in order of appearance); structures of the crosslinkers used for the kinetic studies (FIG. 6b); and a conversion of model peptide to the bicyclic product using crosslinkers 3a to 3e at various time points (FIG. 6c).

FIG. 7 illustrates a LC-MS (280 nm) trace of the reaction mixture of model peptide P1 with BC-TFP (3a). The results show 91% conversion of model peptide to bicyclic product 2a after 10 min incubation, and full conversion within 50 minutes.

FIG. 8 illustrates LC-MS (280 nm) trace of the reaction mixture of model peptide P1 and BC-OSu (3b). The results show 94% conversion of model peptide to bicyclic product 2a after 10 min incubation, and full conversion within 50 minutes.

FIGS. 9a-9c. Monitoring the intermediate for peptide bicyclization. FIG. 9a. LC-MS (280 nm) trace of model peptide cyclization P1 with BC-DFP (3c). The results show the rapid generation of intermediate 2-Int and relatively slow transformation to bicyclic product. 92% of conversion to bicyclic product was obtained after 290 min incubation. FIG. 9b. ESI⁺-MS spectrum of the intermediate 2-Int. Figure discloses SEQ ID NO: 87. FIG. 9c. The percentage of starting peptide P1, monocyclic peptide intermediate (2-Int) and bicyclic peptide (2a) versus reaction time.

FIGS. 10a-10d. FIG. 10a. LC-MS (280 nm) trace of the reaction mixture of model peptide P1 and DBB-OSu 3d. The results show 74% conversion to intermediate Br-Int and 5% of bicyclic product 2b were obtained after 10 min incubation. 30% Conversion to bicyclic product was observed after 6.5 hours. FIG. 10b. ESI⁺-MS spectrum of the intermediate Br-Int. FIG. 10c. ESI⁺-MS spectrum of the bicyclic peptide 2b. FIG. 10d. The percentage of linear peptide (P1), monocyclic peptide intermediate (Br-Int) and bicyclic peptide (2b) versus reaction time.

FIGS. 11a-11c. FIG. 11a. LC-MS (280 nm) trace of the reaction mixture of model peptide P1 and DCA-OSu 3e. The results show 10 minutes reaction gave 27% conversion to monocyclic product CI-Int. 46% of conversion to monocyclic product was observed after 3.5 hours. The bicyclic product was not observed. The 50 min chromatogram showed a slight shift of the peaks due to a LC-MS system glitch. The molecualr identify of the peaks were confirmed by the mass-spec data. FIG. 11b. ESI⁺-MS spectrum of the monocyclic peptide intermediate CI-Int. FIG. 11c. The percentage of linear peptide (P1), monocyclic peptide intermediate (CI-Int) and bicyclic peptide (2c) versus reaction time.

FIGS. 12a-12b. FIG. 12a. LC-MS (280 nm) trace of model peptide P1 cyclization with 3f. The results show quantitative conversion to bicyclic product within 10 min incubation. FIG. 12b. ESI⁺-MS spectrum of the major peak of the reaction mixture (10 min incubation). The observed product belongs to the cyclic peptide 2d. Figure discloses SEQ ID NO: 95.

FIG. 13. LC-MS (280 nm) trace of coupling reaction of model peptide P1-disulfide with 3g at various time points. The results show that the lysine side chain didn't react with tetrafluorophenol ester 3g in the absence of chlorooxime moiety. Note: The peak at 20 min retention time belongs to small molecules from crosslinker decomposition.

FIGS. 14a-14c. FIG. 14a. LC-MS (280 nm) trace of the starting peptide P1-R. Figure discloses SEQ ID NO: 69. FIG. 14b. LC-MS (280 nm) trace of the P1-R peptide modified with BS-OSu (3b). The result shows 86% conversion of the starting peptide to cyclic product. Figure discloses SEQ ID NO: 70. FIG. 14c. ESI⁺-MS spectrum of the major peak of the reaction mixture. The observed peak corresponds to the monocyclic peptide 5i with OSu intact. Figure discloses SEQ ID NO: 70.

FIG. 15. Hydrolysis of OSu ester (S18) in 80% acetone/PBS (pH 7.4) monitored by ¹H NMR.

FIG. 16. Hydrolysis of OSu ester (S18) in 50% acetone/PBS (pH 7.4) monitored by ¹H NMR.

FIGS. 17a-17b. FIG. 17a. LC-MS (280 nm) trace of the peptide P1 bicyclization with 3b. The result shows 95% conversion of the starting peptide to bicyclic product 2a. FIG. 17b. ESI⁺-MS spectrum of the major peak of the reaction mixture. The observed peak corresponds to the bicyclic peptide 2a.

FIGS. 18a-18c. FIG. 18a. LC-MS (280 nm) trace of the peptide P2. Note: the minor peak exhibits the same mass as the major, likely originating from an amino acid racimerization during peptide synthesis. FIG. 18b. LC-MS (280 nm) trace of the P2 bicyclization with 3b. The result shows 97% total conversion of the starting peptide to bicyclic product. Note: the minor product peaks might result from the minor isomer of starting peptide P2. FIG. 18c. ESI⁺-MS spectrum of the major peak of the reaction mixture. The observed peak corresponds to the bicyclic peptide 4a.

FIGS. 19a-19c. FIG. 19a. LC-MS (280 nm) trace of the peptide P3. FIG. 19b. LC-MS (280 nm) trace of the P3 bicyclization with BC-TFP (3a). The result shows 92% conversion of the starting peptide to bicyclic product. FIG. 19c. ESI⁺-MS spectrum of the major peak of the reaction mixture. The observed peak corresponds to the bicyclic peptide.

FIGS. 20a-20b. FIG. 20a. LC-MS (280 nm) trace of the peptide P4 bicyclization with BC-OSu (3b). The result shows 95% conversion of the starting peptide to bicyclic product. Figure discloses SEQ ID NO: 79. FIG. 20b. ESI⁺-MS spectrum of the major peak of the reaction mixture. The observed peak corresponds to the bicyclic peptide 4c. Figure discloses SEQ ID NO: 79.

FIGS. 21a-21c. FIG. 21a. LC-MS (280 nm) trace of the peptide P5. FIG. 21b. LC-MS (280 nm) trace of the P5 bicyclization with BC-TFP (3a). The result shows 94% total conversion of the starting peptide to bicyclic product. *The minor peak presumably corresponds to an isomer of amino acid racemerzation in P5, for which a broadend park was obsered in FIG. 21a. FIG. 21c. ESI⁺-MS spectrum of the major peak of the reaction mixture. The observed peak corresponds to the bicyclic peptide.

FIGS. 22a-22c. FIGS. 22a & 22b. LC-MS (280 nm) trace of the peptide P6 bicyclization with BC-OSu (3b) for 10 min and 4 hours, respectively. The result shows 35% (incubation for 10 min) and 76% (incubation for 4 hours) conversion of the starting peptide to bicyclic product. FIG. 22c. ESI⁺-MS spectrum of the major peak of the reaction mixture. The observed peak corresponds to the bicyclic peptide.

FIGS. 23a-23c. FIG. 23a. LC-MS (280 nm) trace of the starting peptide P7. FIG. 23b. LC-MS (280 nm) trace of the P7 bicyclization with BC-TFP (3a). The result shows 89% conversion of the starting peptide to bicyclic product. FIG. 23c. ESI⁺-MS spectrum of the major peak of the reaction mixture. The observed peak corresponds to the bicyclic peptide 4f.

FIGS. 24a-24f. FIG. 24a. LC-MS (280 nm) trace of the starting peptide P8. Figure discloses SEQ ID NO: 71. FIGS. 24b & 24c. LC-MS (280 nm) trace of the P7 bicyclization with BC-OSu (3b) for 10 min and 20 hours, respectively. The result shows 27% (10 min incubation) and 45% (20 hours incubation) conversion of the starting peptide to bicyclic product. FIG. 24B discloses SEQ ID NO: 71. FIG. 24d. ESI⁺-MS spectrum of the bicyclic peptide 4g. Figure discloses SEQ ID NO: 71. FIG. 24e. ESI⁺-MS spectrum of the reaction intermediate 4g-OSu. Figure discloses SEQ ID NO: 71. FIG. 24f. ESI⁺-MS spectrum of the reaction intermediate 4g-OH.

FIGS. 25a-25c. FIG. 25a. LC-MS (280 nm) trace of the starting peptide P9. FIG. 25b. LC-MS (280 nm) trace of the P8 bicyclization with BC-TFP (3a). The result shows 96% conversion of the starting peptide to bicyclic product. FIG. 25c. ESI⁺-MS spectrum of the major peak of the reaction mixture. The observed peak corresponds to the bicyclic peptide 4g.

FIGS. 26a-26c. FIG. 26a. LC-MS (280 nm) trace of the P10. Figure discloses SEQ ID NO: 72. FIG. 26b. LC-MS (280 nm) trace of the P10 bicyclization with BC-TFP (3a). The result shows 90% total conversion of the starting peptide to bicyclic product. FIG. 26c. ESI⁺-MS spectrum of the major peak of the reaction mixture. The observed peak corresponds to the bicyclic peptide 5a.

FIGS. 27a-27c. FIG. 27a. LC-MS (280 nm) trace of the starting peptide P11. FIG. 27b. LC-MS (280 nm) trace of the P11 bicyclization with BC-TFP (3a). The result shows 93% conversion of the starting peptide to bicyclic product. FIG. 27c. ESI⁺-MS spectrum of the major peak of the reaction mixture. The observed peak corresponds to the bicyclic peptide.

FIGS. 28a-28c. FIG. 28a. LC-MS (280 nm) trace of the starting peptide P12. FIG. 28b. LC-MS (280 nm) trace of the P12 bicyclization with BC-OSu (3b). The result shows 87% total conversion of the starting peptide to bicyclic product. FIG. 28c. ESI⁺-MS spectrum of the major peak of the reaction mixture. The observed peak corresponds to the bicyclic peptide.

FIGS. 29a-29d. FIG. 29a. LC-MS (280 nm) trace of the starting peptide P13. Figure discloses SEQ ID NO: 73. FIG. 29b. LC-MS (280 nm) trace of the P13 bicyclization with BC-OSu (3a). The result shows 64% conversion of the starting peptide to bicyclic product. 24% of TFP intermediate 5d-TFP was also observed. FIG. 29c. ESI⁺-MS spectrum of the major peak of the reaction mixture. The observed peak corresponds to the bicyclic peptide 5d. FIG. 29d. ESI⁺-MS spectrum of the intermediate 5d-TFP. Figure discloses SEQ ID NO: 102.

FIGS. 30a-30b. FIG. 30a. LC-MS (280 nm) trace of the P14 bicyclization with 3a. The result shows 94% conversion of the starting peptide to bicyclic product. Figure discloses SEQ ID NO: 74. FIG. 30b. ESI⁺-MS spectrum of the major peak of the reaction mixture. The observed peak corresponds to the bicyclic peptide 5e. Figure discloses SEQ ID NO: 74.

FIGS. 31a-31c. FIG. 31a. LC-MS (280 nm) trace of the starting peptide P15. FIG. 31b. LC-MS (280 nm) trace of the P15 bicyclization with BC-TFP (3a). The result shows 75% total conversion of the starting peptide to bicyclic product. Note: two peaks of identical mass was observed. The exact origin of the isomers needs further exploration. It could be due to amino acid racemerization or topological isomer formation of the peptide bicycles. FIG. 31c. ESI⁺-MS spectrum of the major peak of the reaction mixture. The observed peak corresponds to the bicyclic peptide.

FIGS. 32a-32d. a) LC-MS (280 nm) trace of the starting peptide P16. **denotes an unidentificable column contamination. FIG. 32b. LC-MS (280 nm) trace of the P16 bicyclization with BC-OSu (3b) for 10 minutes. The result shows over 90% total conversion of the starting peptide to bicyclic product. Four peaks of identical mass were observed, presumably with each pair corresponding to 5g-1 and 5g-2 respectively. The exact origin of the isomers needs further exploration, possibly due to amino acid racemerization or formation of topological isomers of the peptide bicycles. FIGS. 32c & 32d. ESI⁺-MS spectrum of the major peak of the reaction mixture at 15.7 and 16.3 min resepctively.

FIGS. 33a-33c. FIG. 33a. LC-MS (280 nm) trace of the starting peptide P11-R. FIG. 33b. LC-MS (280 nm) trace of the P11-R bicyclization with 3b in PBS/acetonitrile (80/20). The result shows 96% conversion of the starting peptide to bicyclic product. FIG. 33c. ESI⁺-MS spectrum of the major peak of the reaction mixture. The observed peak corresponds to the bicyclic peptide 5h.

FIGS. 34a-34b. FIG. 34a. LC-MS (280 nm) trace of the starting peptide P11-R. FIG. 34b. LC-MS (280 nm) trace of the P11-R bicyclization with 3b in PBS/acetonitrile/DMF (80/17/3). The result shows 93% conversion of the starting peptide to bicyclic product 5h.

FIGS. 35a-35c. FIG. 35a. LC-MS (280 nm) trace of peptide P14 obtained by TCEP reduction. FIG. 35b. LC-MS (280 nm) trace of the reaction mixture of P14 with BC-TFP (3b). The result shows 83% conversion of the starting peptide to bicyclic product. FIG. 35c. ESI⁺-MS spectrum of the major peak of the reaction mixture. The observed product belongs to the bicyclic peptide.

FIGS. 36a-36f. FIG. 36a. LC-MS (280 nm) trace of the starting peptide P1. Figure discloses SEQ ID NO: 75. FIG. 36b. LC-MS (280 nm) trace of the P1 bicyclization with 3b in PBS/acetonitrile (80/20). The result shows 88% conversion of the starting peptide to bicyclic product 2a. Figure discloses SEQ ID NO: 76. FIG. 36c. LC-MS (280 nm) trace of the P1 bicyclization with 3b in PBS/acetonitrile (90/10). The result shows 77% conversion of the starting peptide to bicyclic peptide 2a. Figure discloses SEQ ID NO: 75. FIG. 36d. LC-MS (280 nm) trace of the P1 bicyclization with 3b in PBS/DMF (99/1). The result shows 87% conversion of the starting peptide to bicyclic peptide 2a. Figure discloses SEQ ID NO: 76. FIG. 36e. LC-MS (280 nm) trace of the P1 bicyclization with 3b in PBS/DMSO (99/1). The result shows 100% conversion of the starting peptide to bicyclic peptide 2a. Figure discloses SEQ ID NO: 75. FIG. 36f. ESI⁺-MS spectrum of the major peak of the reaction mixture. The observed peak corresponds to the bicyclic peptide 2a. Figure discloses SEQ ID NO: 76.

FIGS. 37a-37c. FIG. 37a. LC-MS (280 nm) trace of the bicyclic peptide 5h in PBS pH 5.1 over 16 days. FIG. 37b. LC-MS (280 nm) trace of the bicyclic peptide 5h in PBS pH 7.4 over 16 days. FIG. 37c. LC-MS (280 nm) trace of the bicyclic peptide 5h in PBS pH 9.21 over 16 days. The results revealed no change of the LC-MS profile over time under all pH tested, showcasing the robust chemical stability of the bicyclic peptide.

FIG. 38. Stability studies of 5h (0.96 mM) in DMSO-d₆/PBS (pH 5.9)/D₂O (1/8/1) monitored by ¹H NMR. The results reveal no change of NMR profile after 1 week.

FIG. 39 illustrates the plasmid sequencing results highlighting the insertion of TEV cleavage site and an exemplary peptide. Figure discloses SEQ ID NOS 77-79, respectively, in order of appearance.

FIG. 40 illustrates a final construct.

FIGS. 41a-41b illustrate Deconvoluted mass (FIG. 41a) and crude mass envelope (FIG. 41b) of SSL11 protein.

FIGS. 42a-42b illustrate Deconvoluted mass (FIG. 42a) and crude mass envelope (FIG. 42b) of the reduced SSL11 protein (6a).

FIGS. 43a-43b illustrate Deconvoluted mass (FIG. 43a) and crude mass envelope (FIG. 43b) of SSL11 protein after modification with 3b. The observed mass belongs to the SSL11 protein (6b) with a single site modification.

FIGS. 44a-44c. FIGS. 44a-44b illustrate Deconvoluted mass (FIG. 44a) and crude mass envelope (FIG. 44b) of SSL11 protein fragment (6c) after TEV cleavage. The observed mass belongs to the SSL11 protein fragment with surface-exposed lysine and N-terminus unmodified. FIG. 44c. ESI⁺-MS spectrum of the peptide fragment (6d) after TEV cleavage. Figure discloses SEQ ID NO: 80.

FIGS. 45a-45b illustrate Deconvoluted mass (FIG. 45a) and crude mass envelope (FIG. 45b) of reduced SSL11 fusion protein 6a after treatment with 3a. The fully conversion to bicyclization was observed. The major mass peak belongs to bicyclic protein.

FIG. 46 shows cleaved ACX₇C peptide sequence: ACSWGIEQRCGGGENLYFQ (SEQ ID NO: 3) of the N-terminus of the ACX₇C phage variant. Figure discloses SEQ ID NOS 81-82, respectively, in order of appearance.

FIG. 47 shows Cleaved AX₃CX₇C peptide sequence: AGSACSWGIEQRCGGGENLYFQ (SEQ ID NO: 4) of the N-terminus of the AX₃CX₇C phage variant. Figure discloses SEQ ID NOS 83-84, respectively, in order of appearance.

FIG. 48 shows chemical structure of the AX₃CX₇C peptide unmodified, bicyclic, and hydrolyzed. Figure discloses SEQ ID NOS 85, 103, and 86, respectively, in order of appearance.

FIGS. 49a-49d. FIG. 49a. LC-MS TIC trace of the unmodified ACX₇C peptide from phage after TEV cleavage. FIG. 49b. Extracted [M+2H]²⁺ mass range of the unmodified peptide, m/z 1058-1059. FIG. 49c. Extracted [M+2H]²⁺ mass range of the bicyclic peptide if the phage was modified with 3b, m/z 1152-1153. FIG. 49d. Extracted [M+2H]²⁺ mass range of the hydrolyzed peptide if the phage was modified with 3b, m/z 1161-1162.

FIGS. 50a-50d. FIG. 50a. LC-MS TIC trace of the modified ACX₇C peptide from phage after TEV cleavage. FIG. 50b. Extracted [M+2H]²⁺ mass range of the unmodified peptide, m/z 1058-1059. FIG. 50c. Extracted [M+2H]²⁺ mass range of the bicyclic peptide modified with 3b, m/z 1152-1153. FIG. 50d. Extracted [M+2H]²⁺ mass range of the hydrolyzed peptide, m/z 1161-1162.

FIGS. 51a-51d. FIG. 51a. LC-MS TIC trace of the unmodified AX₃CX₇C peptide from phage after TEV cleavage. FIG. 51b. Extracted [M+2H]²⁺ mass range of the unmodified peptide, m/z 1166-1167. FIG. 51c. Extracted [M+2H]²⁺ mass range of the bicyclic peptide if the phage was modified with 3b, m/z 1260-1261. FIG. 51d. Extracted [M+2H]²⁺ mass range of the hydrolyzed peptide if the phage was modified with 3b, m/z 1269-1270. *Mass spectrometry artifact.

FIGS. 52a-52d. FIG. 52a. LC-MS TIC trace of the modified AX₃CX₇C peptide from phage after TEV cleavage. FIG. 52b. Extracted [M+2H]²⁺ mass range of the unmodified peptide, m/z 1166-1167. FIG. 52c. Extracted [M+2H]²⁺ mass range of the bicyclic peptide modified with 3b, m/z 1260-1261. FIG. 52d. Extracted [M+2H]²⁺ mass range of the hydrolyzed peptide, m/z 1269-1270. *Mass spectrometry artifact.

FIGS. 53a-53d. FIG. 53a. LC-MS TIC trace of the modified AX₃CX₇C peptide from phage after TEV cleavage. FIG. 53b. Extracted [M+2H]²⁺ mass range of the unmodified peptide, m/z 1166-1167. FIG. 53c. Extracted [M+2H]²⁺ mass range of the bicyclic peptide modified with aged, partially hydrolyzed 3b, m/z 1260-1261. FIG. 53d. Extracted [M+2H]²⁺ mass range of the hydrolyzed peptide, m/z 1269-1270. *Mass spectrometry artifact.

FIG. 54. Binding curve of bicyclized NP8 to the spike protein determiend from an ELISA assay. Curve fitting yielded a Kd value of 3 μM.

FIG. 55. Binding curve of bicyclized NP10 to the spike protein determiend from an ELISA assay. Curve fitting yielded a Kd value of 400 nM.

### DETAILED DESCRIPTION

The present disclosure provides a cysteine-directed proximity-driven crosslinking strategy for bicyclizing native peptides. Initiated with rapid cysteine labeling followed by proximity-driven acylation of amines, peptide bicycles with varied ring topology are constructed with high efficiency. The utility of this method is demonstrated by bicyclizing peptides fused to proteins as well to the M13 phage, paving the way to phage display of novel peptide bicycles.

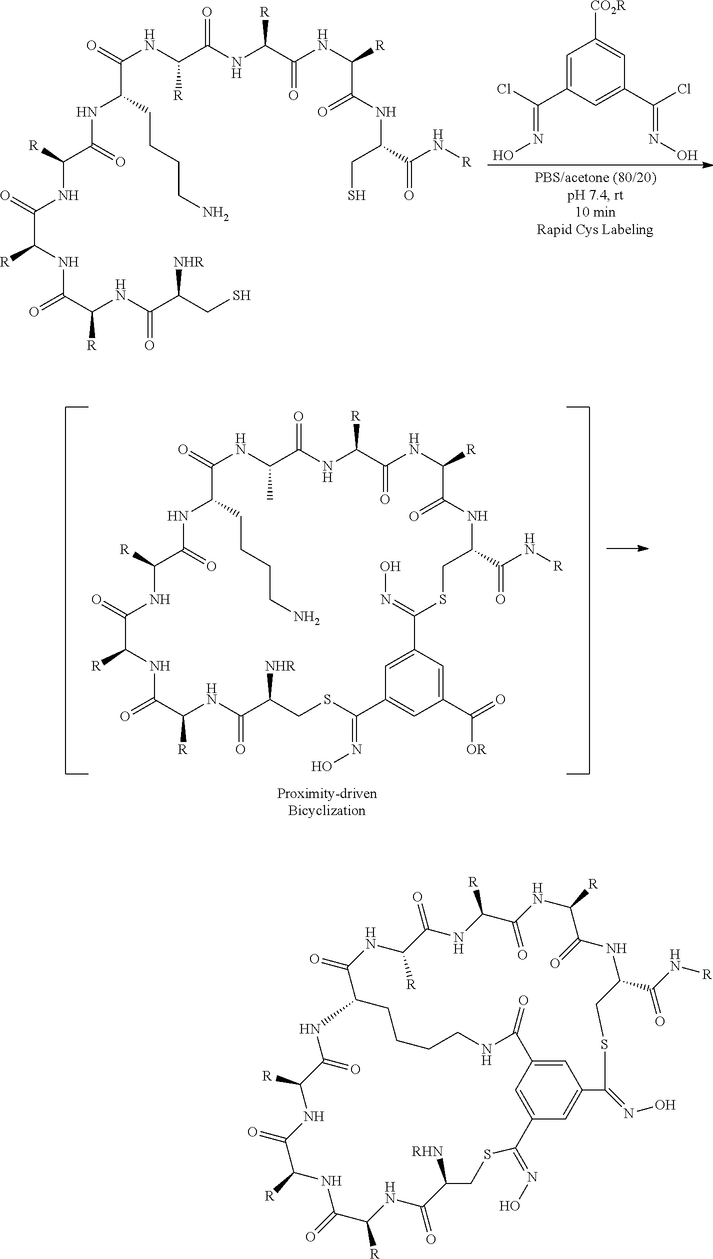

In certain embodiments, the present disclosure provides a cysteine-directed proximity-driven strategy for the constructing bicyclic peptides from simple natural peptide precursors. This linear to bicycle transformation initiates with rapid cysteine labeling, which then triggers proximity-driven amine-selective cyclization. This bicyclization proceeds rapidly under physiologic conditions, yielding bicyclic peptides with a Cys-Lys-Cys, Lys-Cys-Lys or N-terminus-Cys-Cys stapling pattern. The present disclosure further provides the utility and power of this strategy by constructing bicyclic peptides fused to proteins as well as to the M13 phage, paving the way to phage display of novel bicyclic peptide libraries.

The present disclosure also provides novel phage libraries of bicyclic peptides and method of use thereof for screening therapeutic proteins. The present disclosure includes the following document, the content of which is incorporated by reference herewith in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a monomer,” “a catalyst,” or “a polymer,” includes, but is not limited to, mixtures or combinations of two or more such monomers, catalysts, or polymers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

The present disclosure provides a cysteine-directed proximity-driven strategy for native peptide bicyclization using novel chlorooxime-based crosslinkers. The peptide bicyclization was enabled by rapid Cys conjugation, followed by proximity-driven intramolecular amide bond formation. The protocol features fast kinetics, high efficiency, and biocompatible reaction conditions. In certain embodiments, a wide range of backbone and/or sidechain bicyclized peptides were constructed, including N-terminus-Cys-Cys, Cys-Lys-Cys, and Lys-Cys-Lys stapling. The site-specific bicyclization of a peptide-protein fusion highlighted the exquisite site selectivity of this cysteine-directed cyclization.

The present disclosure further provides the efficient bicyclization of peptides displayed on bacteriophage, which paves the way to construct genetically encoded bicyclic peptide libraries. In comparison to previously known methods for phage bicyclization,^{[9-12]} the method disclosed herein avoids the use of engineered cysteine-free phage, harsh oxidation conditions, as well as the generation of bicyclic isomers. These advantages allow the disclosed method to be easily adopted by other laboratories. Note that the disclosed method of bicyclization would require the avoidance of lysines in the peptide substrates, a drawback that can be mitigated by the use of arginines. Further, the disclosed method can be expanded to include additional reactive residues (e.g., histidine, tyrosine and aspartate) as cyclization anchors, which would greatly expand the structural diversity of native peptide cyclization. Display of such libraries on bacteriophage would empower and accelerate molecular discovery as biological probes as well as potential therapeutics.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### General Information

### 1.1 General Information for Reagents and Methods

All Fmoc-protected amino acids, N,N,N′,N′-Tetramethyl-O-(1H-benzotriazol-1-yl)uranium hexafluorophosphate, O-(Benzotriazol-1-yl)-N,N,N′,N′-tetramethyluronium hexafluorophosphate (HBTU), amino acids were purchased from Chem-Impex International (Wood Dale, IL) or Advanced Chemtech (Louisville, KY). Fmoc-Rink Amide MBHA resin was purchased from Novabiochem (San Diego, CA). D-Biotin was purchased from Fisher Scientific. 5-Carboxyfluorescein was purchased from J&K scientific (Beijing, China). Peptide synthesis was carried out on a Tribute peptide synthesizer (Protein Technologies, Tucson, AZ). Peptides were purified on a Waters PrepLC System using a Phenomenex Jupiter C18 column (Torrance, CA). LC-MS spectrometry data were collected using an Agilent 6230 LC TOF mass spectrometer. LC-MS data were processed using Agilent MassHunter software package. Deconvoluted mass data was processed using mMass 5.5.0 and MagTran 1.03 b3 software. ¹H NMR and ¹³C NMR spectra were collected using a VNMRS 400 MHz, 500 MHz or 600 MHz NMR spectrometer. NMR data were processed using MestReNova 14.1.2 software. ¹H and ¹³C chemical shifts were referenced to internal solvent resonances and reported relative to SiMe₄; multiplicities are indicated by s (singlet), d (doublet), t (triplet), q (quartet), m (multiplet) and br (broad). Coupling constants, J, are reported in Hz (Hertz). Mass spectrometry data were collected using an Agilent 6230 LC TOF mass spectrometer.

### 1.2 LC-MS Analysis Methods

Solvent compositions used are 0.1% formic acid, 5% acetonitrile in H₂O (Solvent A) and 0.1% formic acid, 5% H₂O in acetonitrile (Solvent B).

### Method A:

Column: Agilent Poroshell 120 EC-C18 column: 3.0×50 mm, 2.7 μm

### Gradient:

TABLE 1
Gradient for LC-MS Method A

| Time (min) | Solvant A (%) | Solvent B (%) | Flow (m [symbol] /min) |
|---|---|---|---|
| 0.00 | 100 | 0 | 0.200 |
| 5.00 | 100 | 0 | 0.200 |
| 20.00 | 0 | 100 | 0.200 |
| 25.00 | 0 | 100 | 0.200 |
| 26.00 | 100 | 0 | 0.200 |
| 30.00 | 100 | 0 | 0.200 |

[symbol]

indicates data missing or illegible when filed

MS conditions: positive electrospray ionization (ESI) extended dynamic mode in mass range 100-2000 m/z, temperature of drying gas=325° C., flow rate of drying gas=8 L/min, pressure of nebulizer gas=35 psi, the capillary=4.286 uA, fragmentor=175 V, octupole rf voltages=750.

### Method B:

Column: Aeris™ 3.6 μm WIDEPORE XB-C8 200 Å, LC Column 100×4.6 mm, Ea
Gradient:

TABLE 2
Gradient for LC-MS Method B

| | | | |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

indicates data missing or illegible when filed

MS conditions: positive electrospray ionization (ESI) extended dynamic mode in mass range 100-2000 m/z, temperature of drying gas=325° C., flow rate of drying gas=8 L/min, pressure of nebulizer gas=35 psi, the capillary=0.059 uA, fragmentor=175 V, octupole rf voltages=750 V.

### Method C:

Column: Agilent Poroshell 120 EC-C18 column: 3.0×50 mm, 2.7 μm
Gradient:

TABLE 3
Gradient for LC-MS Method C

| | | | |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

indicates data missing or illegible when filed

MS conditions: positive electrospray ionization (ESI) extended dynamic mode in mass range 100-2000 m/z, temperature of drying gas=325° C., flow rate of drying gas=8 L/min, pressure of nebulizer gas=35 psi, the capillary=4.286 uA, fragmentor=175 V, octupole rf voltages=750.

### 1.3 General Procedure for Solid Phase Peptide Synthesis

All peptides were synthesized using standard Fmoc-based Solid Phase Peptide Synthesis (SPPS) chemistry with the Rink Amide MBHA resin as the solid support. All coupling reactions were carried out on a 0.1 mmol scale and used commercially available Fmoc-protected amino acids building blocks. For peptide elongation, the resin was shaken with Fmoc-protected amino acids (5 eq), HBTU (5 eq), 0.40 M N-methylmorpholine in DMF. After each coupling reaction, the Fmoc group was cleaved using 20% piperidine in DMF. The alloc group was removed by the treatment of the resin with Ph(PPh₃)₄/PhSiH₃ in DCM. The cleavage of peptides from resin and the globally deprotection were carried out with Reagent B (a solution of 9.5 mL of TFA, 0.5 mL H₂O, 0.25 mL TIPS and 700 mg of phenol) at room temperature for 3 hours. The reaction mixture was filtered and precipitated out by the addition of cold ethyl ether, and the obtained crude product was purified by RP-HPLC (Waters Prep LC, Jupiter C18 Column) using acetonitrile/H₂O (with 0.1% trifluoroacetic acid) as the gradient elution. The purity of all peptides was determined by LC-MS analysis. The pure peptides were obtained as a solid compound after lyophilization.

TABLE 4
Sequences of Peptides

| | | | | Calc. m/z | Obs. m/z |
|---|---|---|---|---|---|
| Entry | Peptide | SEQ ID NO: | Peptide sequence | [M + 2H]²⁺ | [M + 2H]²⁺ |
| 1 | P1 | 5 | (Ac)CAAAKAAACW-NH₂ | 503.73 | 503.69 |
| 2 | P2 | 6 | (Ac)CAKACW-NH₂ | 722.30 | 722.28 |
| | | | | ([M + H]⁺) | ([M + H]⁺) |
| 3 | P3 | 7 | (Ac)CYKSCW-NH₂ | 830.33 | 830.33 |
| 4 | P4 | 8 | (Ac)CAAAAKAAAACW-NH₂ | 574.76 | 574.77 |
| 5 | P5 | 9 | (Ac)CAEAKAEACW-NH₂ | 561.73 | 561.64 |
| 6 | P6 | 10 | (Ac)CAHAKAHACW-NH₂ | 569.75 | 569.69 |
| 7 | P7 | 11 | (Ac)CAKAHQAACW-NH₂ | 565.25 | 565.27 |
| 8 | P8 | 12 | (Ac)CKCW-NH₂ | 580.23 | 580.21 |
| | | | | ([M + H]⁺) | ([M + H]⁺) |
| 9 | P9 | 13 | (Ac)KACEFCW-NH₂ | 927.38 | 927.34 |
| | | | | ([M + H]⁺) | ([M + H]⁺) |
| 10 | P10 | 14 | GLIGCPFPASWC-NH₂ | 625.29 | 625.25 |
| 11 | P11 | 15 | ALIGCPFPASWC-NH₂ | 632.30 | 632.27 |
| 12 | P12 | 16 | ACPFPASWC-NH₂ | 490.70 | 490.68 |
| 13 | P13 | 17 | ACLIPTWGGC-NH₂ | 510.24 | 510.19 |
| 14 | P14 | 18 | PCPFPASWC-NH₂ | 503.71 | 503.75 |
| 15 | P15 | 19 | ACFMQEPLYICG-NH₂ | 687.30 | 687.15 |
| 16 | P16 | 20 | ALIGCESAYKNTAQCW-NH₂ | 878.91 | 878.92 |
| 17 | P1-R | 21 | (Ac)CAAARAAACW-NH₂ | 517.73 | 517.73 |
| 18 | P11-R | 22 | ALIGCPFPARWC-NH₂ | 666.83 | 666.77 |

### Example 2

### Procedure for the Synthesis of Crosslinkers

### 2.1 Synthesis of Crosslinker 3a, 3b, 3c and 3g

The crosslinker precursor S2 to S6 was prepared from commercially available trimethyl 1,3,5-benzenetricarboxylate (S1) following the method previously reported^{[1]}

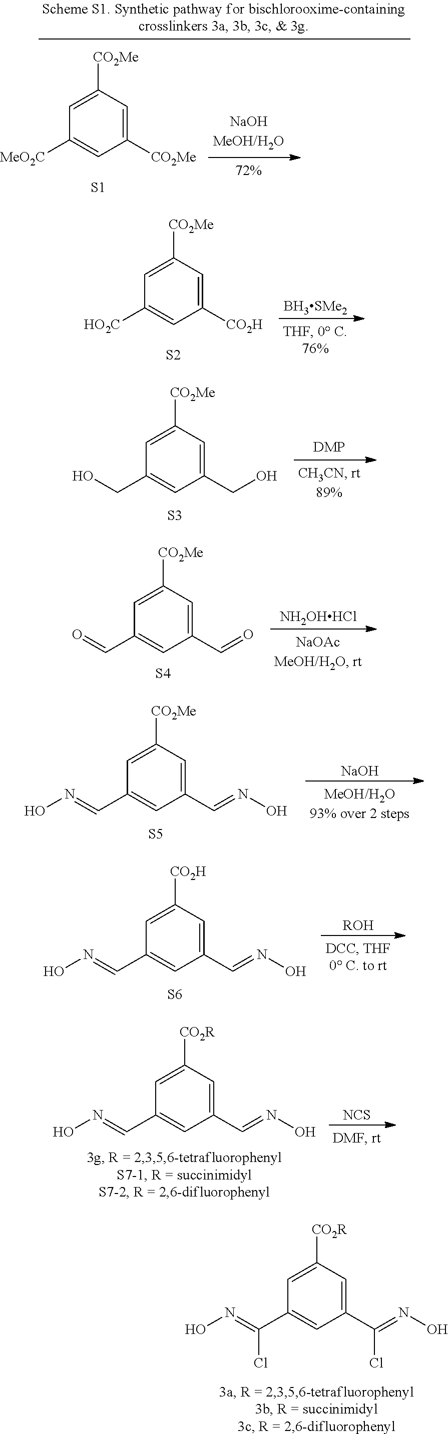

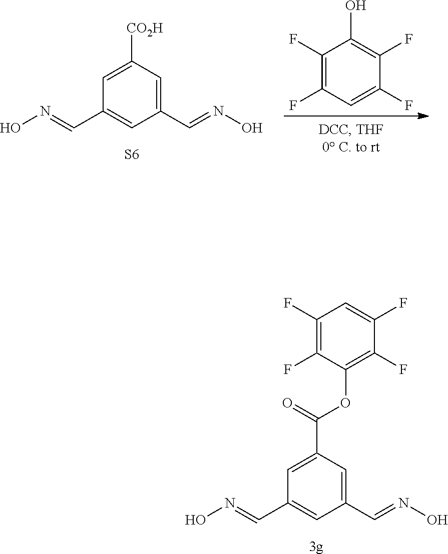

Carboxylic acid S6 (416 mg, 2.0 mmol) was dissolved in 15 mL of dry THF under N₂ and the solution was cooled to 0° C. 2,3,5,6-tetrafluorophenol (364.4 mg, 2.2 mmol) and N,N′-dicyclohexylcarbodiimide (453.9 mg, 2.2 mmol) were added sequentially to the mixture. The mixture was warmed to room temperature slowly and stirred overnight. After filtration, the supernatant was concentrated under reduced pressure. Cold water was added, and the mixture was extracted with ethyl acetate for 3 times and the combined organic layer was washed with sat. NaHCO₃, brine and dried over Na₂SO₄. The residue was purified by flash column chromatography (hexane:ethyl acetate=1:1, R_{f}=0.6, UV). The TFP ester 3g was obtained as a white solid 267 mg, 74%). ¹H NMR (600 MHz, acetone-d₆) δ 10.74 (s, 2H), 8.45 (d, J=1.6 Hz, 2H), 8.35 (s, 2H), 8.29 (t, J=1.7 Hz, 1H), 7.67-7.59 (m, 1H). ¹³C NMR (126 MHz, acetone-d₆) δ 162.76, 147.98, 147.88, 146.13 (dd, J_{C-F}=12.2, 4.4 Hz), 141.60 (dd, J_{C-F}=253.2, 15.5 Hz), 136.10, 130.97, 129.48, 128.91, 105.04 (t, J=23.5 Hz). ¹⁹F NMR (564 MHz, acetone) δ −140.72-−140.81 (m), −154.91 (dd, J=11.7, 7.4 Hz). HRMS (TOF-ESI⁺) m/z calc. for C₁₅H₉F₄N₂O₄ [M+H]⁺ 357.0498, found 357.0305.

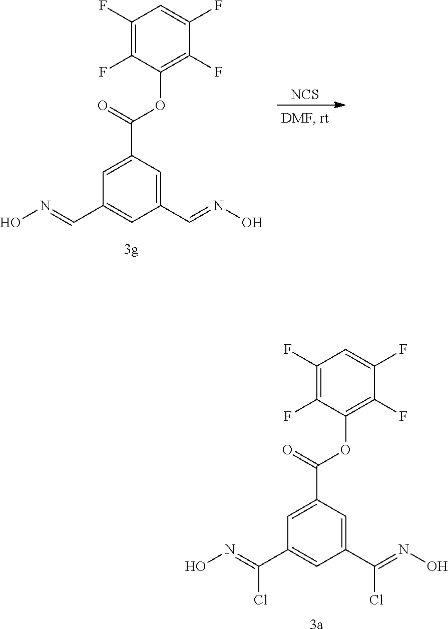

N-Chlorosuccinimide (146.9 mg, 1.1 mmol) was added portion-wise to a stirred solution of oxime 3g (178.0 mg, 0.5 mmol) in DMF (5 mL) at room temperature and rection mixture was stirred overnight. The reaction mixture was diluted with cold 5% LiCl aqueous solution and diethyl ether, the organic layer was separated, and the aqueous phage was extracted with diethyl ether. The combined organic layer was washed with cold 5% LiCl aqueous solution, brine, and dried over anhydrous sodium sulfate. Organic solvent was removed under reduced pressure. The bischlorooxime 3a was obtained as a white solid (212 mg, 99%). ¹H NMR (500 MHz, acetone-d₆) δ 11.91 (s, 2H), 8.71 (s, 2H), 8.68 (s, 1H), 7.68-7.58 (m, 1H). ¹³C NMR (126 MHz, acetone-d₆) δ 162.15, 148.12 (dt, J=12.7, 4.6 Hz), 146.15 (dd, J=12.1, 4.3 Hz), 141.56 (dd, J=248.2, 14.1 Hz), 135.77, 135.65, 131.04, 130.38, 128.99, 105.14 (t, J=23.5 Hz). HRMS (TOF-ESI⁺) m/z calc. for C₁₅H₇Cₗ₂F₄N₂O₄ [M+H]⁺ 424.9719, found 424.9468.

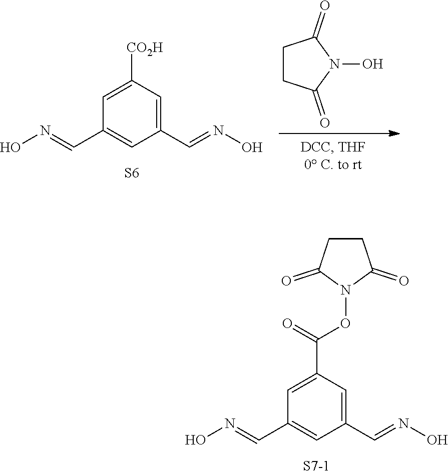

Carboxylic acid S6 (125 mg, 0.6 mmol) was dissolved in 4 mL of dry THF under N₂ and the solution was cooled to 0° C. N-hydroxysuccinimide (82.9 mg, 0.72 mmol) and N,N′-dicyclohexylcarbodiimide (123.8 mg, 0.6 mmol) were added sequentially to the mixture and stirred at 0° C. until completion (monitored by TLC). After filtration, cold water was added, and the mixture was extracted with ethyl acetate for 3 times and the combined organic layer was washed with sat. NaHCO₃, brine and dried over Na₂SO₄. NHS ester S7-1 was obtained as white solid (176 mg, 96%). ¹H NMR (500 MHz, DMSO-d₆) δ 11.62 (s, 2H), 8.33 (s, 2H), 8.26 (s, 2H), 8.24 (s, 1H), 2.91 (s, 4H). ¹³C NMR (125 MHz, DMSO-d₆) δ 170.22, 161.30, 146.78, 135.03, 129.75, 127.84, 125.65, 25.58. HRMS (TOF-ESI⁺) m/z calc. for C₁₃H₁₂N₃O₆ [M+H]⁺ 306.0726, found 306.0681.

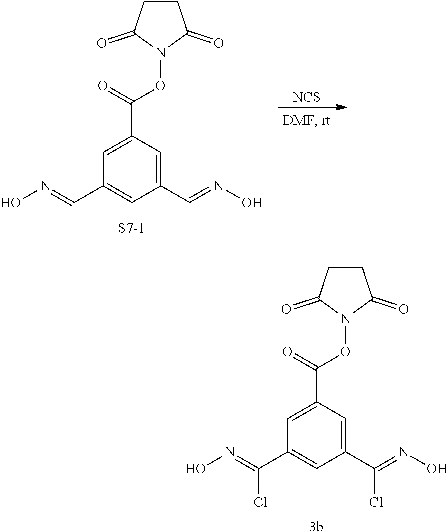

N-Chlorosuccinimide (58.8 mg, 0.44 mmol) was added portion-wise to a stirred solution of oxime S7-1 (61.0 mg, 0.20 mmol) in DMF (2 mL) at room temperature and rection mixture was stirred overnight. The reaction mixture was diluted with cold 5% LiCl aqueous solution and diethyl ether, the organic layer was separated, and the aqueous phage was extracted with diethyl ether. The combined organic layer was washed with cold 5% LiCl aqueous solution, brine, and dried over anhydrous sodium sulfate. Organic solvent was removed under reduced pressure. The bischlorooxime 3b was obtained as a white solid (68 mg, 91%). ¹H NMR (500 MHz, acetone-d₆) δ 11.89 (s, 2H), 8.69 (t, J=1.8 Hz, 1H), 8.61 (d, J=1.8 Hz, 2H), 3.00 (s, 4H). ¹³C NMR (126 MHz, acetone-d₆) δ 170.32, 161.69, 135.66, 135.59, 131.04, 129.90, 127.39, 26.33. HRMS (TOF-ESI⁺) m/z calc. for C₁₃H₁₀Cl₂N₃O₆ [M+H]⁺ 373.9947, found 373.9735.

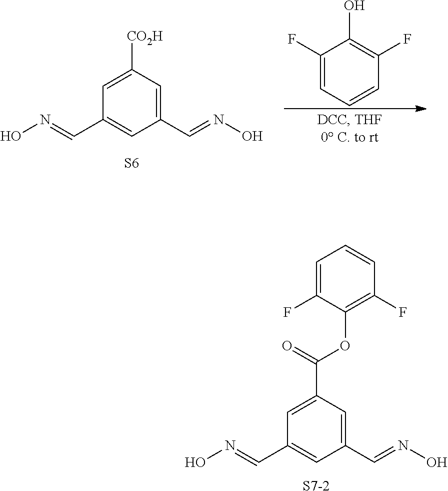

Carboxylic acid S6 (208 mg, 1.0 mmol) was dissolved in 7 mL of dry THF under N₂ and the solution was cooled to 0° C. 2,3,5,6-tetrafluorophenol (143 mg, 1.1 mmol) and N,N′-dicyclohexylcarbodiimide (227 mg, 1.1 mmol) were added sequentially to the mixture. The mixture was warmed to room temperature slowly and stirred overnight. After filtration, the supernatant was concentrated under reduced pressure. Cold water was added, and the mixture was extracted with ethyl acetate for 3 times and the combined organic layer was washed with sat. NaHCO₃, brine and dried over Na₂SO₄. The residue was purified by flash column chromatography (hexane:ethyl acetate=1:1, R_{f}=0.6, UV). The BFP ester S7-2 was obtained as a white solid 87 mg, 27%). ¹H NMR (600 MHz, acetone-d₆) δ 10.76 (s, 2H), 8.44 (d, J=1.1 Hz, 2H), 8.34 (s, 2H), 8.26 (s, 1H), 7.46-7.38 (m, 1H), 7.23 (t, J=8.2 Hz, 2H). ¹³C NMR (151 MHz, acetone-d₆) δ 163.02, 155.82 (dd, J_{C-F}=249.2, 3.9 Hz), 147.77, 147.67, 135.58, 130.23, 129.32, 129.05, 128.03 (t, J_{C-F}=9.2 Hz), 112.93 (dd, J_{C-F}=18.2, 4.0 Hz). HRMS (TOF-ESI⁺) m/z calc. for C₁₅H₁₁F₂N₂O₄ [M+H]⁺ 321.0687, found 321.0505.

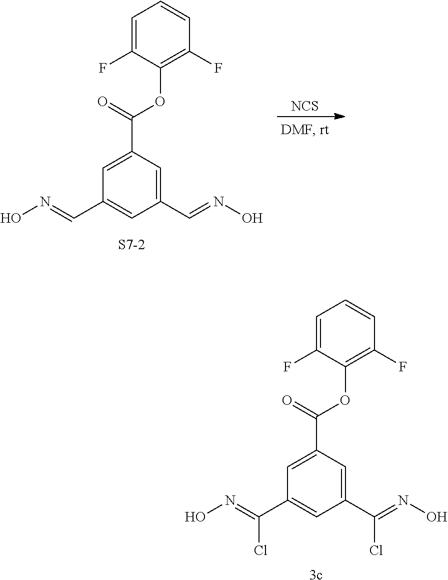

N-Chlorosuccinimide (10 mg, 0.075 mmol) was added portion-wise to a stirred solution of oxime S7-2 (11 mg, 0.034 mmol) in DMF (1 mL) at room temperature and the rection mixture was stirred overnight. The reaction mixture was diluted with cold 5% LiCl aqueous solution and diethyl ether, the organic layer was separated, and the aqueous phage was extracted with diethyl ether. The combined organic layer was washed with cold 5% LiCl aqueous solution, brine, and dried over anhydrous sodium sulfate. Organic solvent was removed under reduced pressure. The bischlorooxime 3c was obtained as a white solid (13 mg, 97%). ¹H NMR (600 MHz, acetone-d₆) δ 11.92 (s, 2H), 8.71 (d, J=1.7 Hz, 2H), 8.67 (t, J=1.7 Hz, 1H), 7.48-7.40 (m, 1H), 7.28-7.21 (m, 2H). ¹³C NMR (151 MHz, acetone-d₆) δ 162.63, 155.92 (dd, J_{C-F}=249.4, 3.8 Hz), 135.69, 135.41, 130.57, 130.10, 129.55, 128.39 (t, J_{C-F}=9.2 Hz), 113.15 (dd, J_{C-F}=18.3, 3.9 Hz). HRMS (TOF-ESI⁺) m/z calc. for C₁₅H₉Cₗ₂F₂N₂O₄ [M+H]⁺ 388.9907, found 388.9682.

### 2.2 Synthesis of Crosslinker 3d and 3e

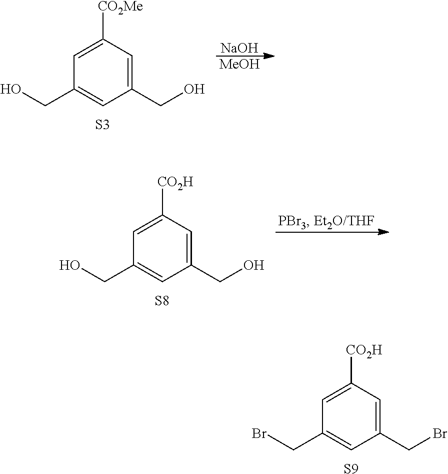

4 mL of 1 M NaOH aqueous solution was added to the solution of ester S3 (392 mg, 2 mmol) in MeOH (5 mL), and the mixture was stirred at room temperature overnight. After concentrated, the residue was diluted with water, acidified to pH 1 with 6 N HCl to precipitate the white solid. The mixture was extracted with ethyl acetate for 3 times and the combined organic layers was washed with brine, dried over anhydrous Na₂SO₄. The organic solvent was removed under reduced pressure. The pure carboxylic acid S8 was obtained as a white solid without further purification (317 mg, 87%). ¹H NMR (600 MHz, CD₃OD) δ 7.94 (d, J=1.7 Hz, 2H), 7.59-7.57 (m, 1H), 4.66 (s, 4H). ¹³C NMR (151 MHz, acetone-d₆) δ 167.84, 143.85, 131.33, 130.01, 127.09, 64.26. HRMS (TOF-ESI⁺) m/z calc. for C₉H₁₁O₄[M+H]⁺ 183.0657, found 183.0546. The mixture of carboxylic acid S8 (104.8 mg, 0.58 mmol), phosphorus tribromide (33.9 μL, 0.357 mmol) in diethyl ether (3 mL) and THF (0.6 mL) was stirred at room temperature for 3 hours. The mixture was diluted with water, extracted with ethyl acetate for 3 times. The combined organic layers were washed with brine, dried over anhydrous Na₂SO₄. The organic solvent was removed under reduced pressure, and the residue was purified by flash column chromatography (ethyl acetate, R_{f}=0.5, UV). The product S9 was obtained as a white solid (58 mg, 32%). ¹H NMR (600 MHz, acetone-d₆) δ 8.06 (s, 2H), 7.81 (s, 1H), 4.74 (s, 4H).¹³C NMR (151 MHz, acetone-d₆) δ 166.59, 140.31, 134.75, 132.47, 130.79, 32.83. HRMS (TOF-ESI⁺) m/z calc. for C₉H₉Br₂O₂ [M+H]⁺ 306.8969, found 306.8785.

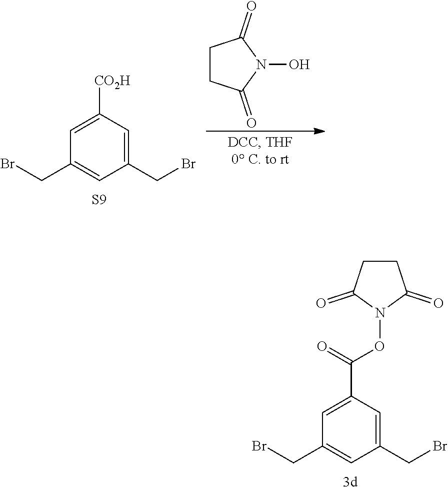

Carboxylic acid S9 (44 mg, 0.14 mmol) was dissolved in 1 mL of dry THF under N₂ and the solution was cooled to 0° C. N-hydroxysuccinimide (17.7 mg, 0.154 mmol) and N,N′-dicyclohexylcarbodiimide (31.8 mg, 0.154 mmol) were added sequentially to the mixture and stirred at 0° C. until completion (monitored by TLC). After filtration, cold water was added, and the mixture was extracted with ethyl acetate for 3 times and the combined organic layer was washed with sat. NaHCO₃, brine and dried over Na₂SO₄. NHS ester 3d was obtained as white solid (56 mg, 99%). ¹H NMR (600 MHz, acetone-d₆) δ 8.16 (d, J=1.1 Hz, 2H), 7.99 (s, 1H), 4.79 (s, 4H), 2.98 (s, 4H). ¹³C NMR (151 MHz, acetone-d₆) δ 170.47, 162.06, 141.51, 137.07, 131.20, 127.32, 32.36, 26.41. HRMS (TOF-ESI⁺) m/z calc. for C₁₃H₁₁Br₂NO₄ [M]⁺ 402.9055, found 403.0070.

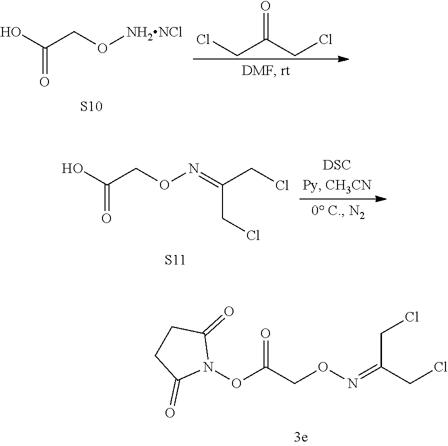

The mixture of amine S10 (2 mmol) with 1,3-dichloropropan-2-one (2.9 mmol, 1.45 equiv.) in DMF (5 mL) was stirred at room temperature. Upon completion (monitored the reaction by TLC), the reaction mixture was diluted with saturated brine solution and extracted with diethyl ether for 3 times. The combined organic phase was washed with saturated brine solution and dried over anhydride Na₂SO₄. The solvent was removed under reduced pressure and the residue was purified by flash column chromatography (hexane:ethyl acetate=1:1 to 1:2). The product S11 was obtained as white solid (340 mg, 85%). ¹H NMR (500 MHz, cdcl₃) δ 4.74 (s, 2H), 4.41 (s, 2H), 4.27 (s, 2H). Pyridine (0.48 mL, 6 mmol) was added to the solution of acid S11 (400 mg, 2 mmol) in dry acetonitrile (20 mL) under nitrogen, and the mixture was cooled to 0° C. with ice bath. N,N′-disuccinimidyl carbonate (DSC) (768 mg, 3 mmol) was added and the mixture was stirred for 6 hours. The solvent was removed under reduced pressure and the residue was dissolved with ethyl acetate (100 mL). The organic phase was washed with cold water (20 mL), cold sat. NaHCO₃ (20 mL), cold brine (20 mL) and dried over anhydride Na₂SO₄. The solvent was removed under reduced pressure. The NHS-ester 3e was obtained as white solid (541 mg, 91%) and used without further purification. ¹H NMR (500 MHz, CDCl₃) δ 4.99 (s, 2H), 4.37 (s, 2H), 4.25 (s, 2H), 2.81 (s, 4H). ¹³C NMR (126 MHz, acetone-d₆) δ 170.32, 161.69, 135.66, 135.59, 131.04, 129.90, 127.39, 26.33. HRMS (TOF-ESI⁺) m/z calc. for C₉H₁₁Cl₂N₂O₅ [M+H]⁺ 297.0045, found 296.9865.

### 2.3 Synthesis of Crosslinker 3h

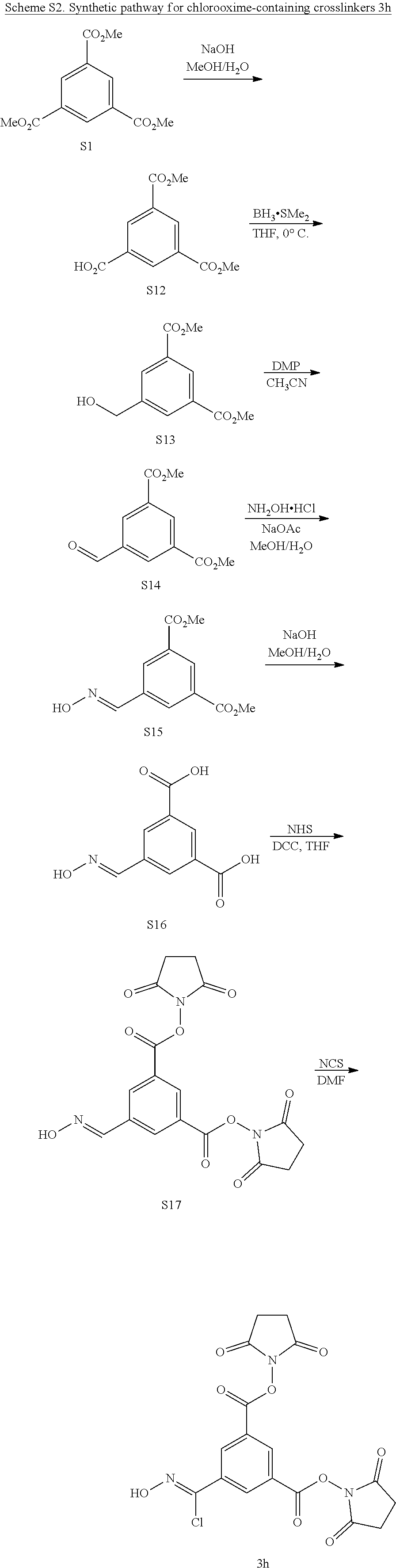

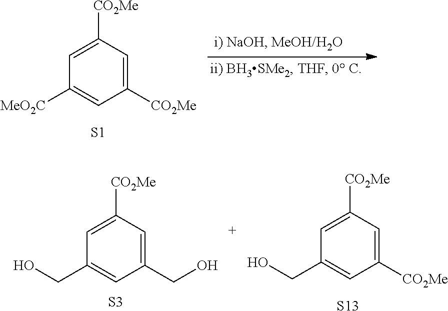

To a suspension of trimethyl benzene-1,3,5-tricarboxylate S1 (5.0 g, 20 mmol) in 300 mL of methanol was added 0.8 M sodium hydroxide aqueous solution (51.8 mL, 2.07 eq.). The cloudy mixture was stirred vigorously, and it turned to a clear solution slowly. The solution was stirred overnight. Upon completion, the solvent was removed under reduced pressure and the residue was dissolved with 100 mL of water. The mixture was acidified with 1 N HCl to pH 2 and extracted with ethyl acetate 3 times. The combined organic layer was washed with water, brine, and dried over anhydrous sodium sulfate. After filtration, the organic solvent was removed under reduced pressure, and the acid was obtained as white solid and directly used for the next step. The acid was dissolved in 80 mL of dry THF, and the solution was cooled to 0° C. under N₂. BH₃·SMe₂ (8.0 mL, 80 mmol) was added dropwise to the solution and the reaction mixture was stirred at 0° C. until the reaction was completed (monitored by TLC). The reaction was quenched with water at 0° C. and concentrated under reduced pressure. The residue was extracted with ethyl acetate for 3 times and the combined organic layer was washed with sat. NaHCO₃, brine and dried over Na₂SO₄. After filtration, organic solvent was removed under reduced pressure and the residue was purified by flash column chromatography (hexane:ethyl acetate=1:2 to ethyl acetate). Product S13 (R_{f}=0.6 in ethyl acetate, UV) was obtained as white solid (1.86 g, 40% over 2 steps), and S3 (R_{f}=0.4 in ethyl acetate, UV) was obtained as white solid (2.40 g, 60% over 2 steps). S13: ¹H NMR (500 MHz, CDCl₃) δ 8.60 (s, 1H), 8.23 (s, 2H), 4.81 (s, 2H), 3.95 (s, 6H). S3: ¹H NMR (500 MHz, CDCl₃) δ 7.95 (s, 2H), 7.61 (s, 1H), 4.76 (s, 4H), 3.93 (s, 3H). HRMS (TOF-ESI⁺) m/z calc. for C₁₁H₁₃O₅ [M+H]⁺ 225.0763, found 225.0696.

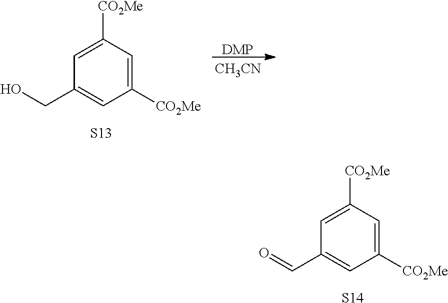

To a solution of alcohol S13 (1.79 g, 8.0 mmol) in 200 mL of CH₃CN was added Dess-Martin periodinance (5.09 g, 12.0 mmol) and the mixture was stirred at room temperature until completion (monitored by TLC). After filtration and the supernatant was concentrated, the residue was purified by flash column chromatography (hexane:ethyl acetate=3:1 to 2:1). Aldehyde S14 was obtained as a white solid (1.59 g, 89%). ¹H NMR (600 MHz, CDCl₃) δ 10.10 (s, 1H), 8.88 (s, 1H), 8.68 (d, J=1.3 Hz, 2H), 3.97 (s, 6H). ¹³C NMR (151 MHz, cdcl3) δ 190.46, 165.24, 136.98, 135.77, 134.37, 131.94, 52.85. HRMS (TOF-ESI⁺) m/z calc. for C₁₁H₁₁O₅ [M+H]⁺ 223.0606, found 223.0535.

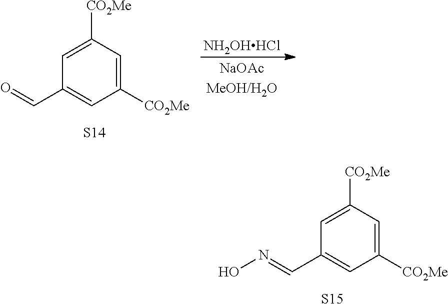 [SMILES: COC(=O)c1cc(C=O)cc(C(=O)OC)c1 ; "NH2OH·HCl" ; "S14" ; COC(=O)c1cc(/C=N/O)cc(C(=O)OC)c1 ; "S15"]

NaOAc·3H₂O (2.45 g, 18 mmol) and hydroxylamine hydrochloride (0.834 g, 12 mmol) were added to a solution of aldehyde S14 (1.33 g, 6 mmol) in 80 mL of methanol and 8 mL of water. The mixture was stirred at room temperature for 1.5 hours. Methanol was removed under reduced pressure and the residue was dissolved in water, extracted with ethyl acetate, washed with brine and dried over Na₂SO₄. After filtration and concentration, the residue was purified by flash column chromatography (hexane:ethyl acetate=2:1, R_{f}=0.3, UV). Oxime S15 was obtained as white solid (1.28 g, 90%). ¹H NMR (500 MHz, acetone) δ 10.71 (s, 1H), 8.53 (t, J=1.6 Hz, 1H), 8.44 (d, J=1.4 Hz, 2H), 8.33 (s, 1H), 3.94 (s, 6H). ¹³C NMR (151 MHz, cdcl₃) δ 190.46, 165.24, 136.98, 135.77, 134.37, 131.94, 52.85. HRMS (TOF-ESI⁺) m/z calc. for C₁₁H₁₂NO₅ [M+H]⁺ 238.0715, found 238.0638.

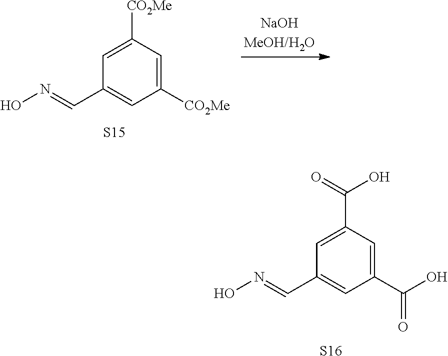 [SMILES: COC(=O)c1cc(/C=N/O)cc(C(=O)OC)c1 ; "S15" ; O=C(O)c1cc(/C=N/O)cc(C(=O)O)c1 ; "S16"]

S15 (1.0 g, 4.2 mmol) was dissolved in 100 mL of methanol and added 50 mL of 1 M NaOH. Upon complete consumption of the oxime (monitored by TLC), the mixture was concentrated under reduced pressure, diluted with water and acidified to pH 2 with 6 N HCl. The mixture was diluted with ethyl acetate and centrifuged to give a white solid. The solid was mixed with acetone and dried under reduced pressure. Pure product S16 was obtained as white solid (0.87 g, 99%). ¹H NMR (600 MHz, acetone) δ 10.72 (s, 1H), 8.65 (s, 1H), 8.51 (s, 2H), 8.36 (s, 1H). ¹³C NMR (151 MHz, acetone) δ 166.60, 148.19, 135.56, 132.65, 132.33, 131.89. HRMS (TOF-ESI⁺) m/z calc. for C₉H₈NO₅ [M+H]⁺ 210.0402, found 210.0303.

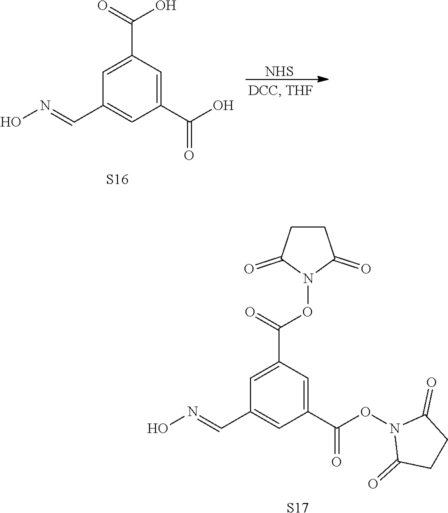 [SMILES: O=C(O)c1cc(/C=N/O)cc(C(=O)O)c1 ; "NHS" ; "S16" ; O=C(ON1C(=O)CCC1=O)c1cc(/C=N/O)cc(C(=O)ON2C(=O)CCC2=O)c1 ; "S17"]

Carboxylic acid S16 (104.5 mg, 0.5 mmol) was dissolved in 4 mL of dry THF under N₂ and the solution was cooled to 0° C. N-hydroxysuccinimide (126.6 mg, 1.1 mmol) and N,N′-dicyclohexylcarbodiimide (206.3 mg, 1.0 mmol) were added sequentially to the mixture and stirred at 0° C. until completion (monitored by TLC). After filtration, cold water was added, and the mixture was extracted with ethyl acetate for 3 times and the combined organic layer was washed with sat. NaHCO₃, brine and dried over Na₂SO₄. After filtration, organic solvent was removed under reduced pressure. 2 mL of ethyl acetate and 30 mL of hexane were added to the residue to precipitate out a white solid. Pure NHS ester S19 was collected by filtration as white solid (174 mg, 86%). ¹H NMR (500 MHz, acetone) b 10.99 (s, 1H), 8.72 (s, 3H), 8.46 (s, 1H), 2.99 (s, 8H). ¹³C NMR (126 MHz, acetone) δ 170.40, 161.68, 147.36, 137.28, 134.26, 132.09, 128.24, 26.54. HRMS (TOF-ESI⁺) m/z calc. for C₁₇H₁₄N₃O₉ [M+H]⁺ 404.0730, found 404.0529.

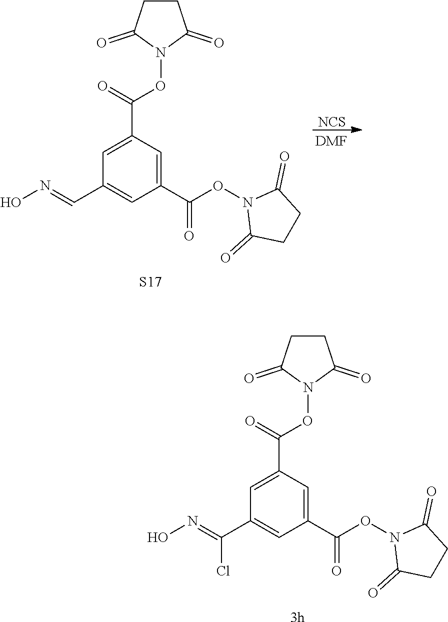

N-chlorosuccinimide (29.4 mg, 0.22 mmol) was added portion-wise to a stirred solution of oxime S17 (80.6 mg, 0.2 mmol) in DMF (1 mL) at room temperature and the rection mixture was stirred overnight. The reaction mixture was diluted with cold 5% LiCl aqueous solution and diethyl ether (white solid formed), the organic layer was separated, and the aqueous phage was extracted with diethyl ether. The combined organic layer was washed with cold 5% LiCl aqueous solution, brine, and dried over anhydrous sodium sulfate. The remaining white solid was dissolved with acetone. The organic phases were combined, and organic solvent was removed under reduced pressure. Acetone was added to the residue and the mixture was concentrated under reduced pressure to remove the water completely. Ether was added to the residue to give fine powder, and the residue was dried slowly by N₂ flow. The powder was further dried under reduced pressure. The product 3h was obtained as a white powder (73 mg, 83%). ¹H NMR (500 MHz, acetone) δ 12.05 (s, 1H), 8.87 (d, J=1.5 Hz, 2H), 8.82 (s, 1H), 3.00 (s, 8H). ¹³C NMR (126 MHz, acetone) δ 170.30, 161.35, 136.55, 135.30, 134.21, 133.11, 128.28, 26.48. HRMS (TOF-ESI⁺) m/z calc. for C₁₇H₁₃ClN₃O₉ [M+H]⁺ 438.0340, found 438.0093.

### Example 3

### Kinetic Studies and Control Experiments

### 3.1 Summary of Kinetic Studies for Peptide Bicyclization

General procedure: 20 μL of crosslinker (100 μM stock solution in acetone) and 80 μL of model peptide (Ac)CAAAKAAACW-NH₂ (SEQ ID NO: 5) (12.5 μM freshly made solution in PBS pH 7.4) were added to a 200 μL vial, resulting 10 μM of peptide and 20 μM of crosslinker. The mixture was pipetted several times and incubated at room temperature. The reaction mixture was analyzed by LC-MS at different time points (Method A; SEQ ID NOS 5 and 68, respectively).

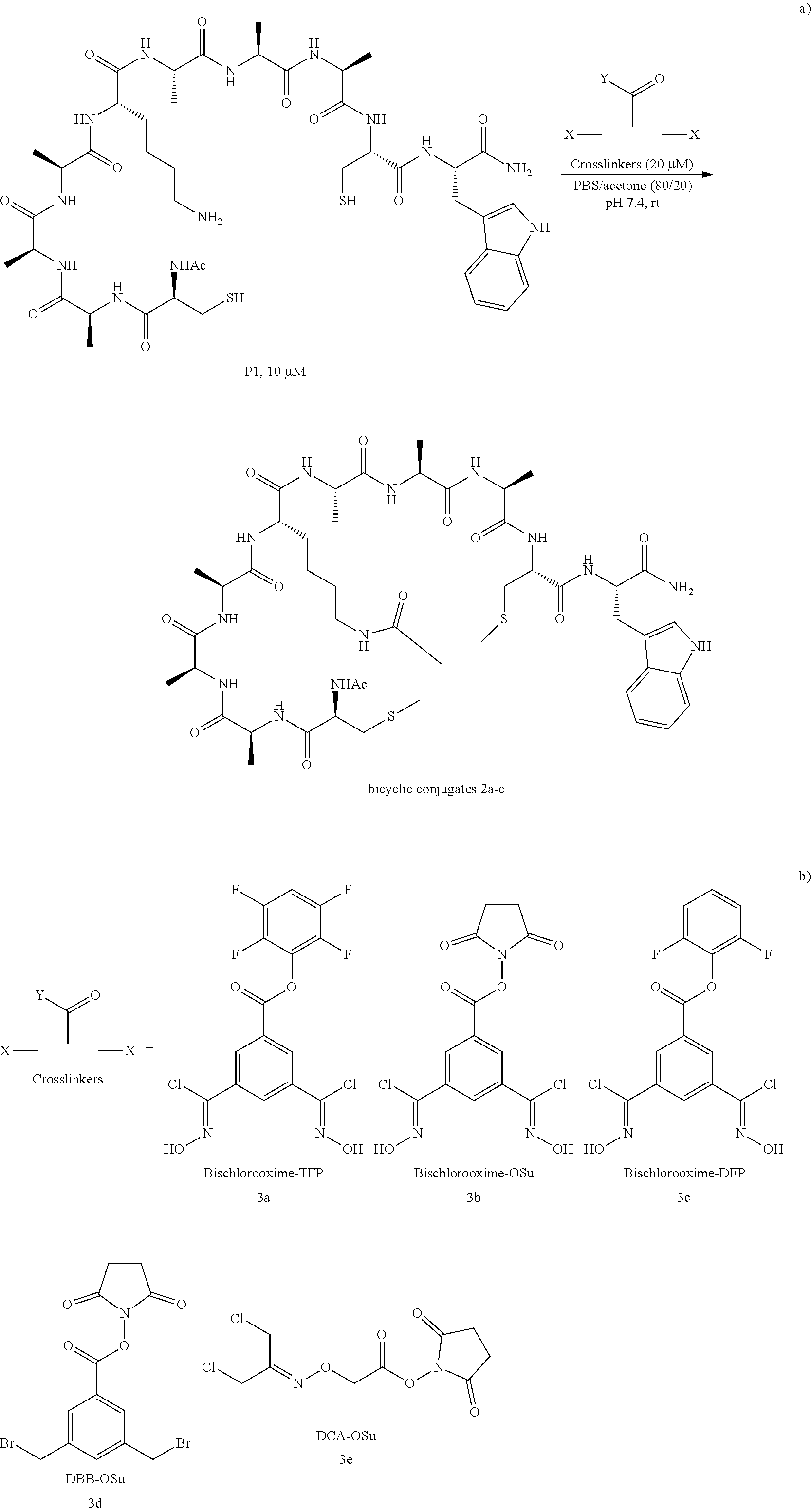

3.1.1 Kinetics of Peptide Bicyclization with BC-TFP (3a); SEQ ID NOS 5 and 42, Respectively.

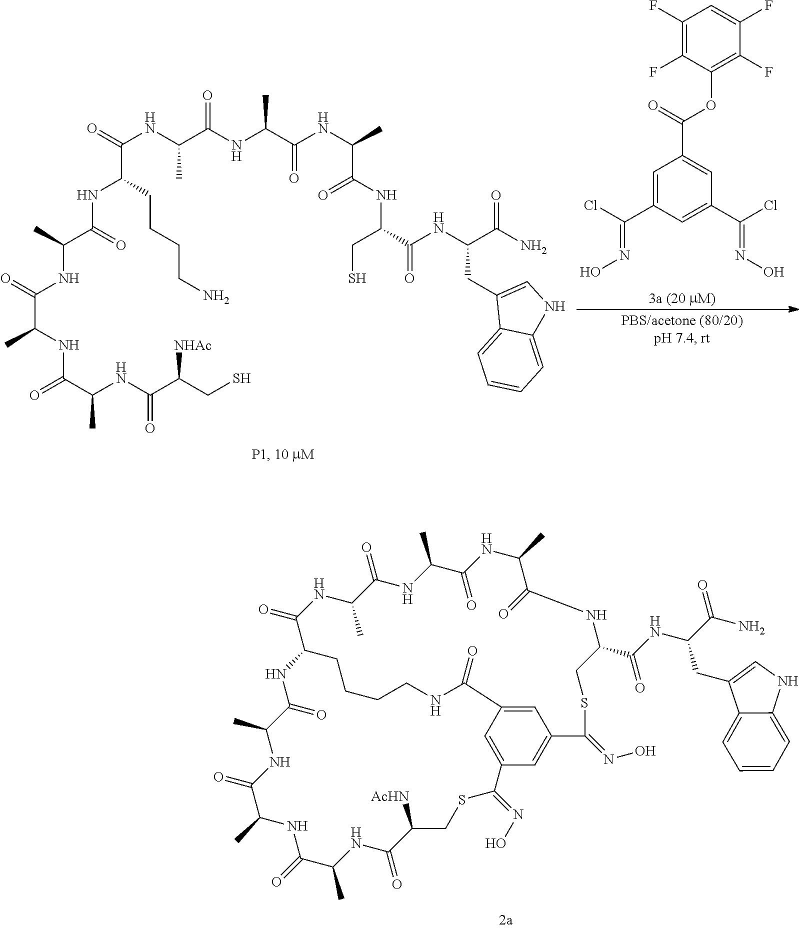 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(Oc1c(F)c(F)cc(F)c1F)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](C)NC(=O)[C@H](CCCCNC3=O)NC(=O)[C@H](C)NC(=O)[C@H](C)NC(=O)[C@H](C)NC1=O]

3.1.2 Kinetics of Peptide Bicyclization with BC-OSu (3b); SEQ ID NOS 5 and 42, Respectively.

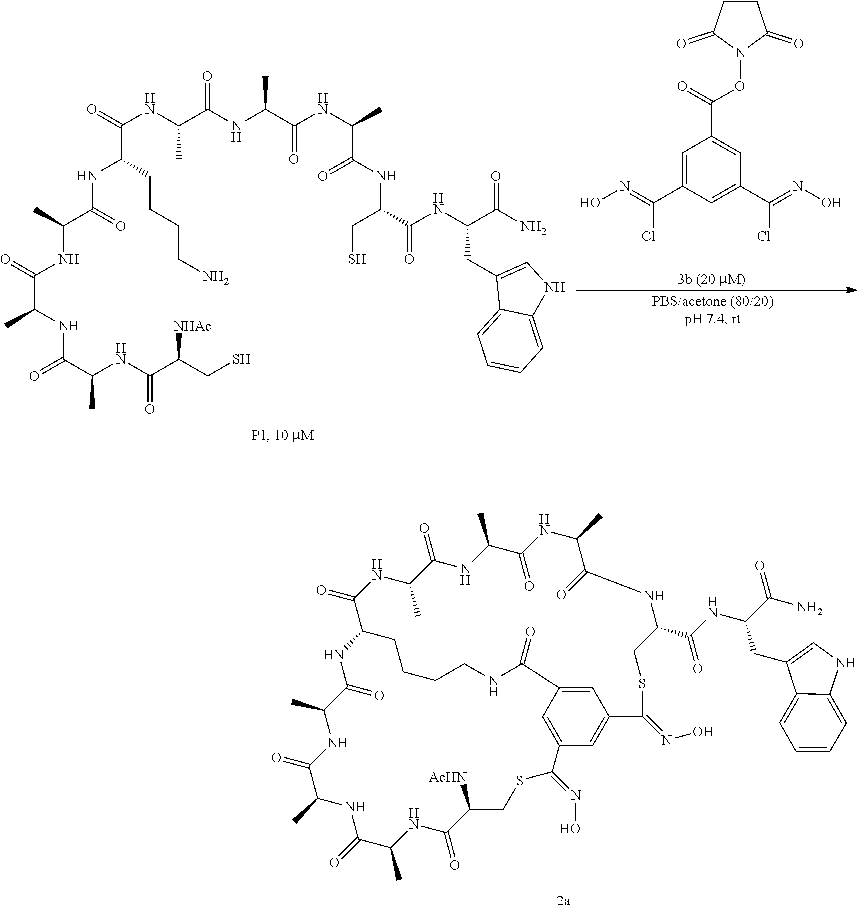 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](C)NC(=O)[C@H](CCCCNC3=O)NC(=O)[C@H](C)NC(=O)[C@H](C)NC(=O)[C@H](C)NC1=O]

3.1.3 Kinetics of Peptide Bicyclization with BC-DFP (3c); SEQ ID NOS 5, 87, and 42, Respectively.

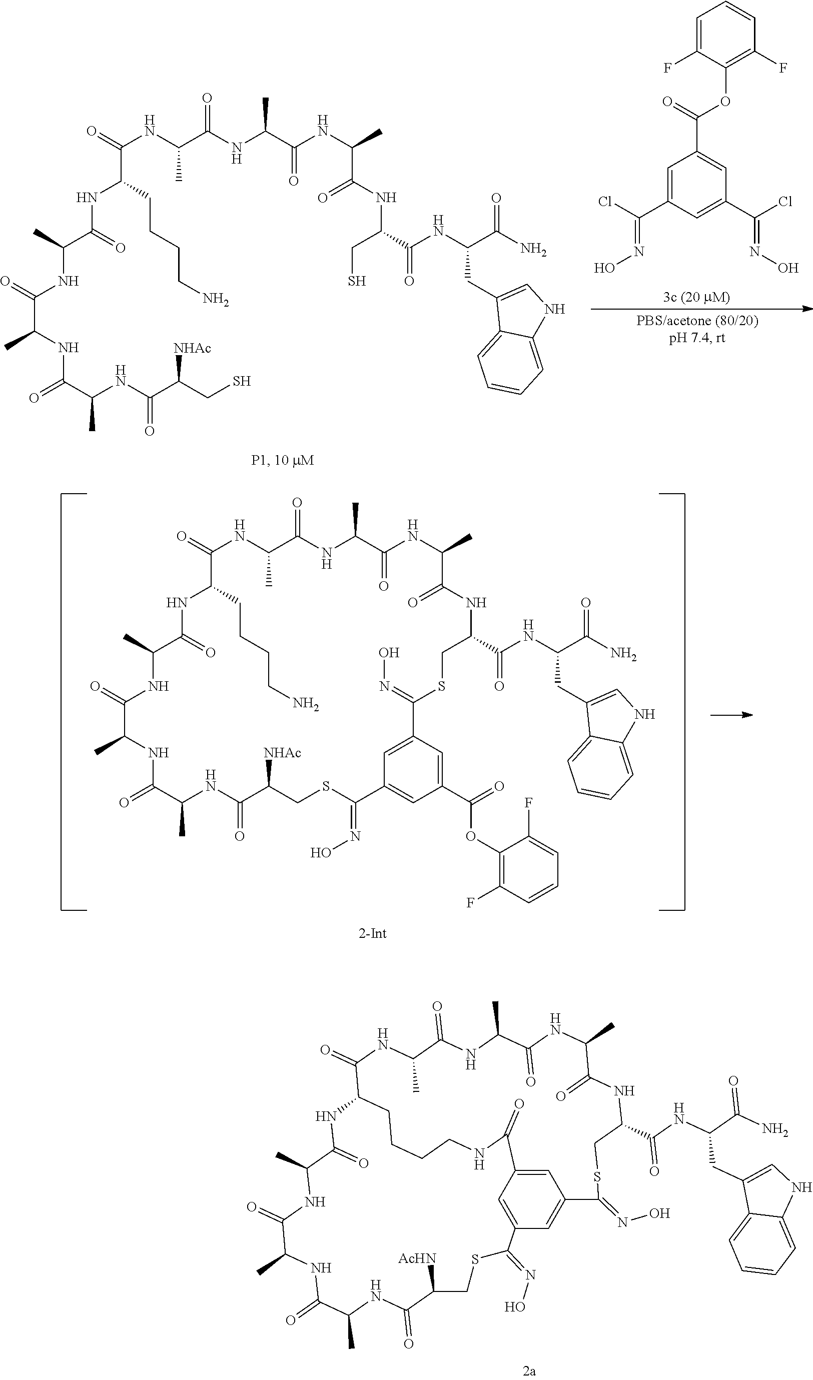

3.1.4 Kinetics of Peptide Bicyclization with DBB-OSu (3d); SEQ ID NOS 5 and 88, Respectively.

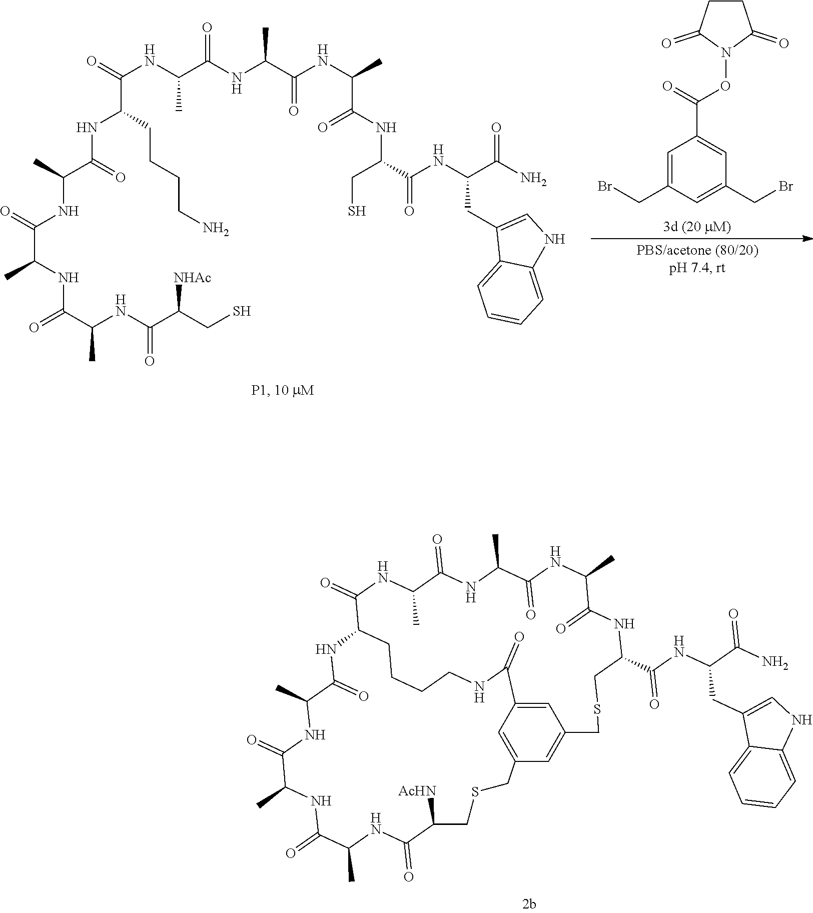 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(CBr)cc(CBr)c1 ; CC(=O)N[C@H]1CSCc2cc3cc(c2)C(=O)NCCCC[C@H](NC(=O)[C@H](C)NC(=O)[C@H](C)NC(=O)[C@H](C)NC1=O)C(=O)N[C@@H](C)C(=O)N[C@@H](C)C(=O)N[C@@H](C)C(=O)N[C@H](C(=O)N[C@@H](Cc1c[nH]c2ccccc12)C(N)=O)CSC3]

### Proposed Structures for Monocyclic Intermediate Br-Int; SEQ ID NOS 89-91, Respectively:

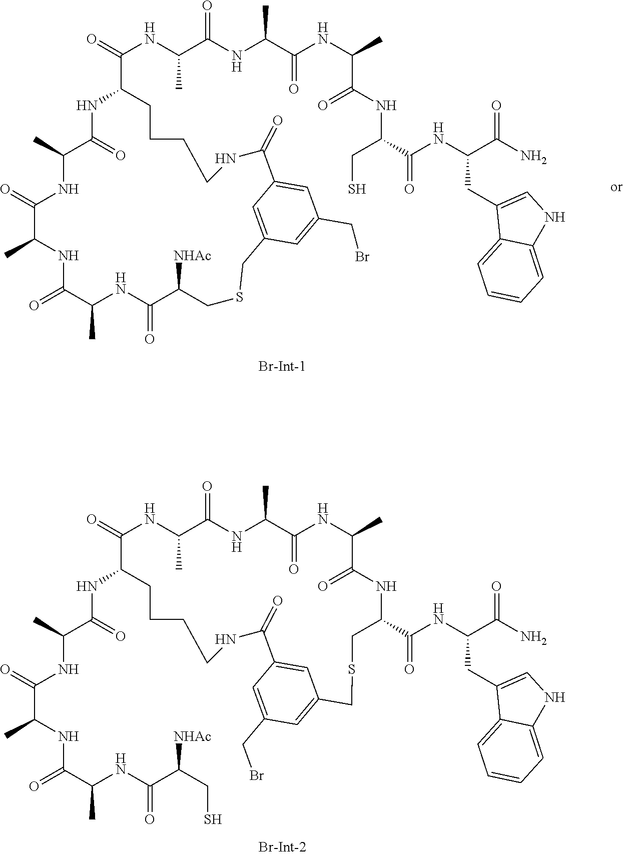 [SMILES: CC(=O)N[C@H]1CSCc2cc(CBr)cc(c2)C(=O)NCCCC[C@@H](C(=O)N[C@@H](C)C(=O)N[C@@H](C)C(=O)N[C@@H](C)C(=O)N[C@@H](CS)C(=O)N[C@@H](Cc2c[nH]c3ccccc23)C(N)=O)NC(=O)[C@H](C)NC(=O)[C@H](C)NC(=O)[C@H](C)NC1=O ; CC(=O)N[C@@H](CS)C(=O)N[C@@H](C)C(=O)N[C@@H](C)C(=O)N[C@@H](C)C(=O)N[C@H]1CCCCNC(=O)c2cc(CBr)cc(c2)CSC[C@@H](C(=O)N[C@@H](Cc2c[nH]c3ccccc23)C(N)=O)NC(=O)[C@H](C)NC(=O)[C@H](C)NC(=O)[C@H](C)NC1=O]

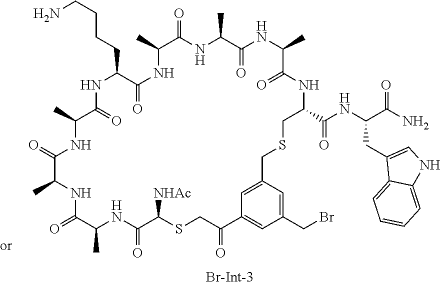 [SMILES: CC(=O)N[C@H]1SCC(=O)c2cc(CBr)cc(c2)CSC[C@@H](C(=O)N[C@@H](Cc2c[nH]c3ccccc23)C(N)=O)NC(=O)[C@H](C)NC(=O)[C@H](C)NC(=O)[C@H](C)NC(=O)[C@H](CCCCN)NC(=O)[C@H](C)NC(=O)[C@H](C)NC(=O)[C@H](C)NC1=O]

3.1.5 Kinetics of Peptide Bicyclization with DCA-OSu (3e); SEQ ID NOS 5 and 43, Respectively.

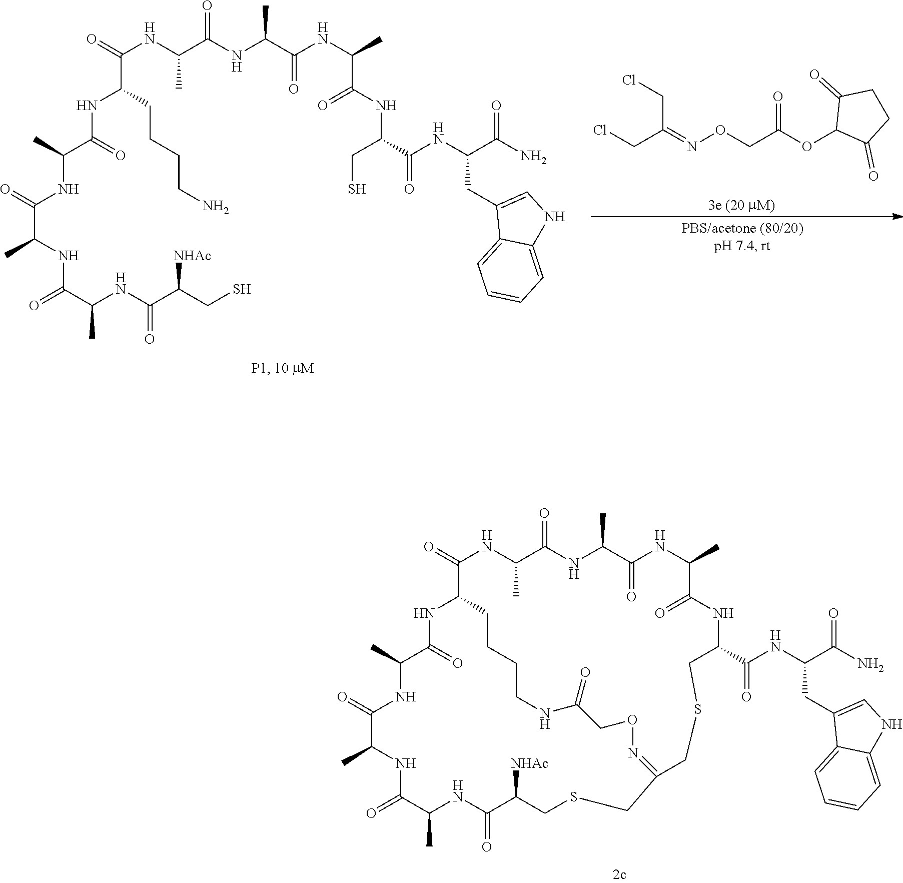 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(CON=C(CCl)CCl)OC1C(=O)CCC1=O ; CC(=O)N[C@H]1CSC/C2=N/OCC(=O)NCCCC[C@H](NC(=O)[C@H](C)NC(=O)[C@H](C)NC(=O)[C@H](C)NC1=O)C(=O)N[C@@H](C)C(=O)N[C@@H](C)C(=O)N[C@@H](C)C(=O)N[C@H](C(=O)N[C@@H](Cc1c[nH]c3ccccc13)C(N)=O)CSC2]

### Proposed Structure for Monocyclic Product CI-Int; SEQ ID NOS 92-94, Respectively:

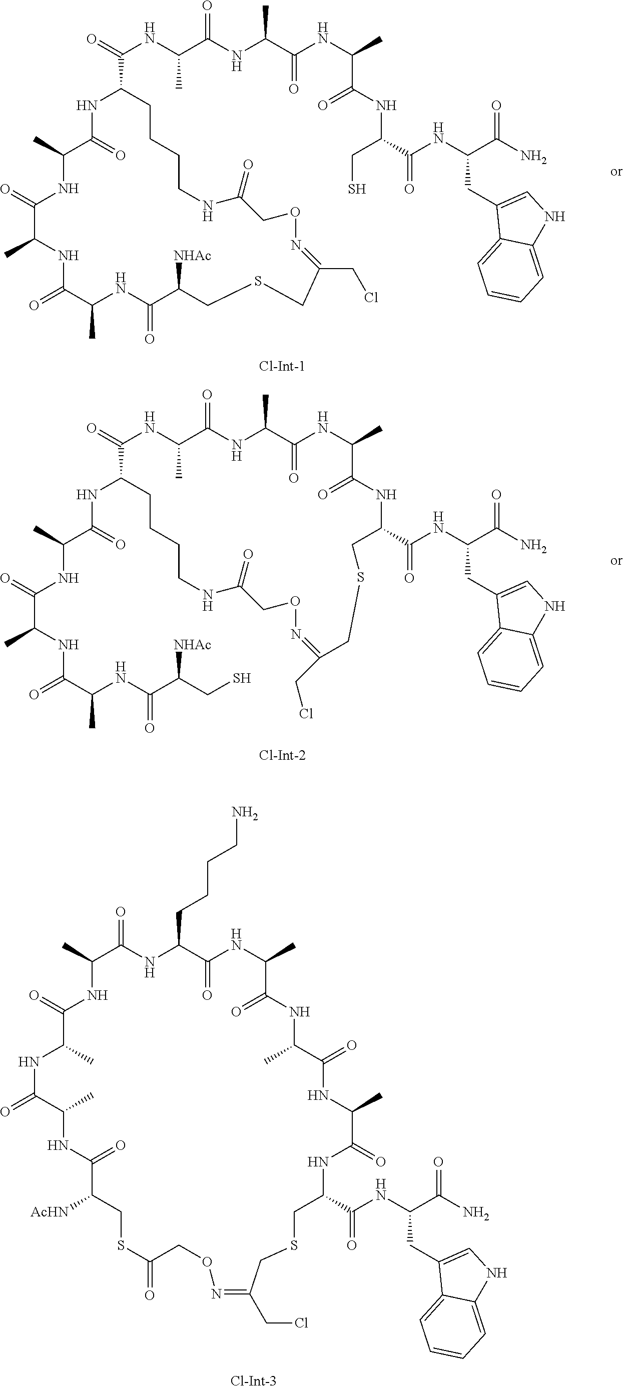

### 3.2 Control Experiments

3.2.1 Peptide Cyclization with Ester-Free Crosslinker 3f; SEQ ID NOS 5 and 95, Respectively.

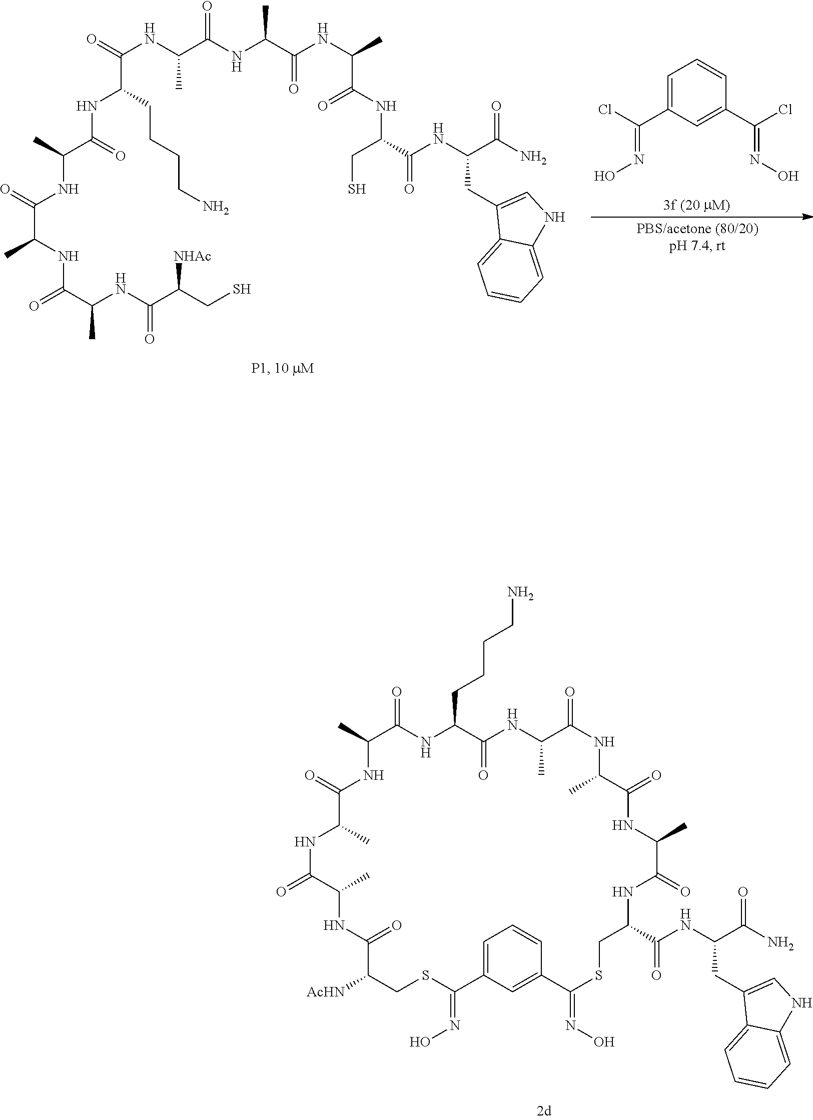 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O/N=C(\Cl)c1cccc(/C(Cl)=N/O)c1 ; CC(=O)N[C@H]1CS/C(=N\O)c2cccc(c2)/C(=N/O)SC[C@@H](C(=O)N[C@@H](Cc2c[nH]c3ccccc23)C(N)=O)NC(=O)[C@H](C)NC(=O)[C@H](C)NC(=O)[C@H](C)NC(=O)[C@H](CCCCN)NC(=O)[C@H](C)NC(=O)[C@H](C)NC(=O)[C@H](C)NC1=O]

20 μL of ester-free bischlorooxime crosslinker 3f (100 μM stock solution in acetone) and 80 μL of model peptide (Ac)CAAAKAAACW-NH₂ (SEQ ID NO: 5) (12.5 μM freshly made solution in PBS pH 7.4) were added to a 200 μL vial, resulting 10 μM of peptide and 20 μM of crosslinker 3e. The mixture was pipetted several times and incubated at room temperature. The reaction mixture was analyzed by LC-MS at different time points (Method A).

3.2.2 Peptide Coupling with Chlorooxime-Free Crosslinker 3g; SEQ ID NOS 96 and 101, Respectively.

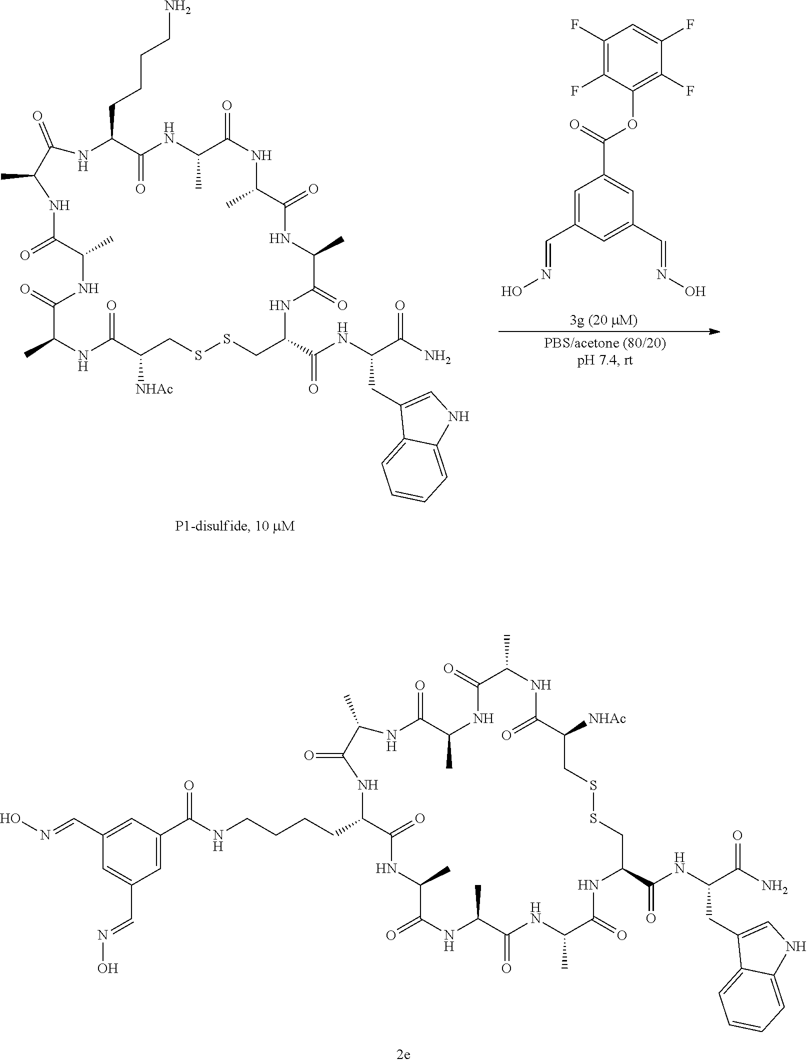 [SMILES: CC(=O)N[C@H]1CSSC[C@@H](C(=O)N[C@@H](Cc2c[nH]c3ccccc23)C(N)=O)NC(=O)[C@H](C)NC(=O)[C@H](C)NC(=O)[C@H](C)NC(=O)[C@H](CCCCN)NC(=O)[C@H](C)NC(=O)[C@H](C)NC(=O)[C@H](C)NC1=O ; O=C(Oc1c(F)c(F)cc(F)c1F)c1cc(/C=N/O)cc(/C=N/O)c1 ; CC(=O)N[C@H]1CSSC[C@@H](C(=O)N[C@@H](Cc2c[nH]c3ccccc23)C(N)=O)NC(=O)[C@H](C)NC(=O)[C@H](C)NC(=O)[C@H](C)NC(=O)[C@H](CCCCNC(=O)c2cc(/C=N/O)cc(/C=N/O)c2)NC(=O)[C@H](C)NC(=O)[C@H](C)NC(=O)[C@H](C)NC1=O]

20 μL of chlorooxime-free tetrafluorophenol crosslinker 3g (100 μM stock solution in acetone) and 80 μL of model peptide with disulfide form (12.5 μM freshly made solution in PBS pH 7.4) were added to a 200 μL vial, resulting 10 μM of peptide and 20 μM of crosslinker. The mixture was pipetted several times and incubated at room temperature. The reaction mixture was analyzed by LC-MS at different time points (Method A).

3.2.3 Modification of Amine-Free (Ac)CAAARAAACW-NH₂ (SEQ ID NO: 21) with BC-OSu (3b); SEQ ID NOS 21 and 97, Respectively.

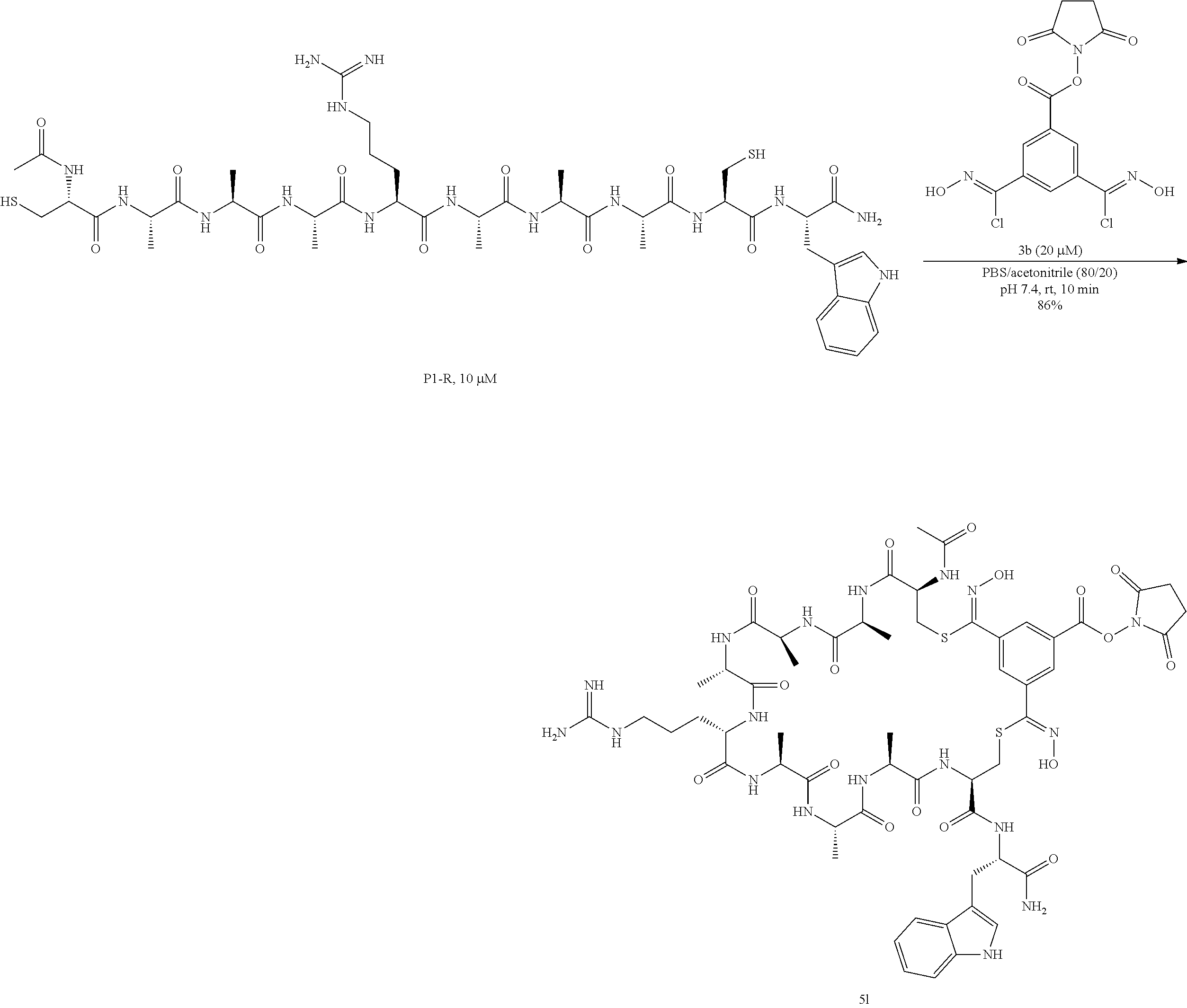 [SMILES: CC(=O)N[C@@H](CS)C(=O)N[C@@H](C)C(=O)N[C@@H](C)C(=O)N[C@@H](C)C(=O)N[C@@H](CCCNC(=N)N)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N/O)c2cc(C(=O)ON3C(=O)CCC3=O)cc(c2)/C(=N/O)SC[C@@H](C(=O)N[C@@H](Cc2c[nH]c3ccccc23)C(N)=O)NC(=O)[C@H](C)NC(=O)[C@H](C)NC(=O)[C@H](C)NC(=O)[C@H](CCCNC(=N)N)NC(=O)[C@H](C)NC(=O)[C@H](C)NC(=O)[C@H](C)NC1=O]

3.2.4 Proposed Mechanism for Peptide Bicyclization with Bischlorooxime Crosslinkers

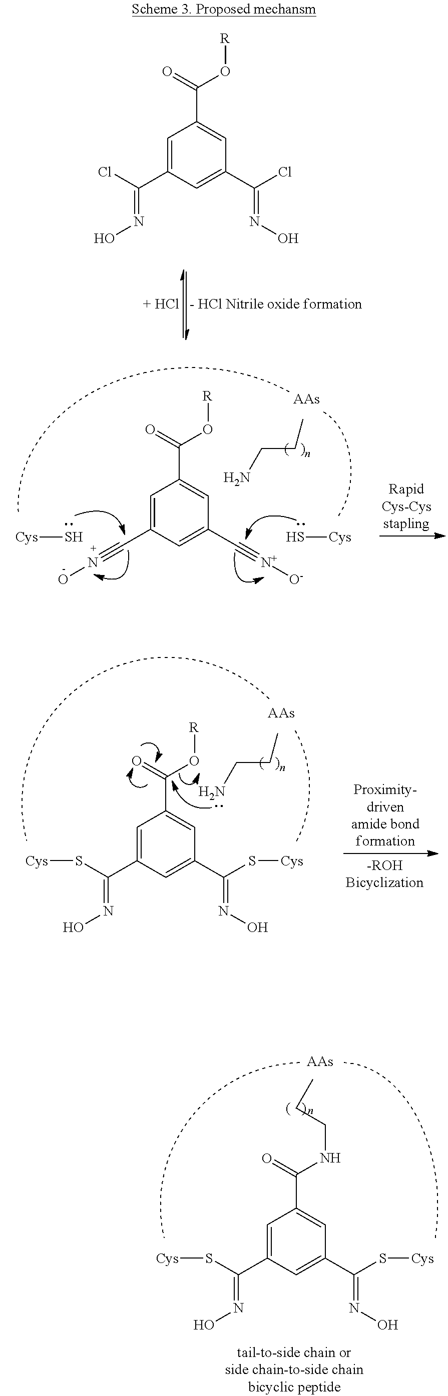

### 3.3 Stability of OSu Ester Towards Hydrolysis

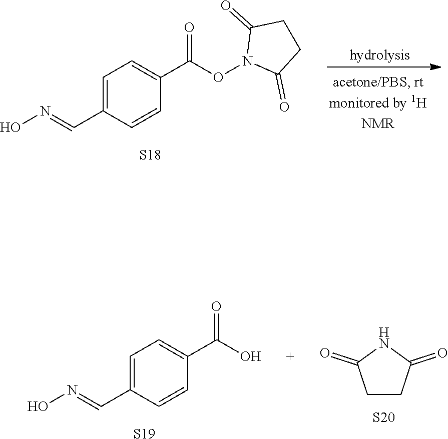

TABLE 5
Hydrolysis of OSu Ester (S18) in Aqueous Solution

| | | | | Percentage |
|---|---|---|---|---|
| Entry | Solvent | Time | | of S20 (%) |
| 1 | acetone-d₆/PBS/D₂O (8/1/1) | 20 | min | trace |
| 2 | acetone-d₆/PBS/D₂O (8/1/1 | 1 | h | trace |
| 3 | acetone-d₆/PBS/D₂O (8/1/1) | 20 | h | 4 |
| 4 | acetone-d₆/PBS/D₂O (5/4/1) | 20 | min | 12 |
| 5 | acetone-d₆/PBS/D₂O (5/4/1) | 1 | h | 26 |
| 6 | acetone-d₆/PBS/D₂O (5/4/1) | 2 | h | 33 |

### Example 4

### Native Peptide Bicyclizations Using Bischlorooxime (BC) Derivatives 4.1 Peptide Bicyclization Through Cys-Lys-Cys Stapling Using BC-TFP (3a) or BC-OSu (3b)

General procedure: 20 μL of bischlorooxime crosslinker 3a or 3b (100 μM stock solution in acetone) and 80 μL of cysteine/lysine-containing peptide (12.5 μM freshly made solution in PBS pH 7.4) were added to a 200 μL vial, resulting 10 μM of peptide and 20 μM of crosslinker. The mixture was pipetted several times and incubated at room temperature for 10 minutes. The reaction mixture was analyzed by LC-MS (Method A).

4.1.1 Bicyclization of (Ac)CAAAKAAACW-NH₂ (SEQ ID NO: 5) with BC-OSu (3b); SEQ ID NOS 5 and 42, Respectively.

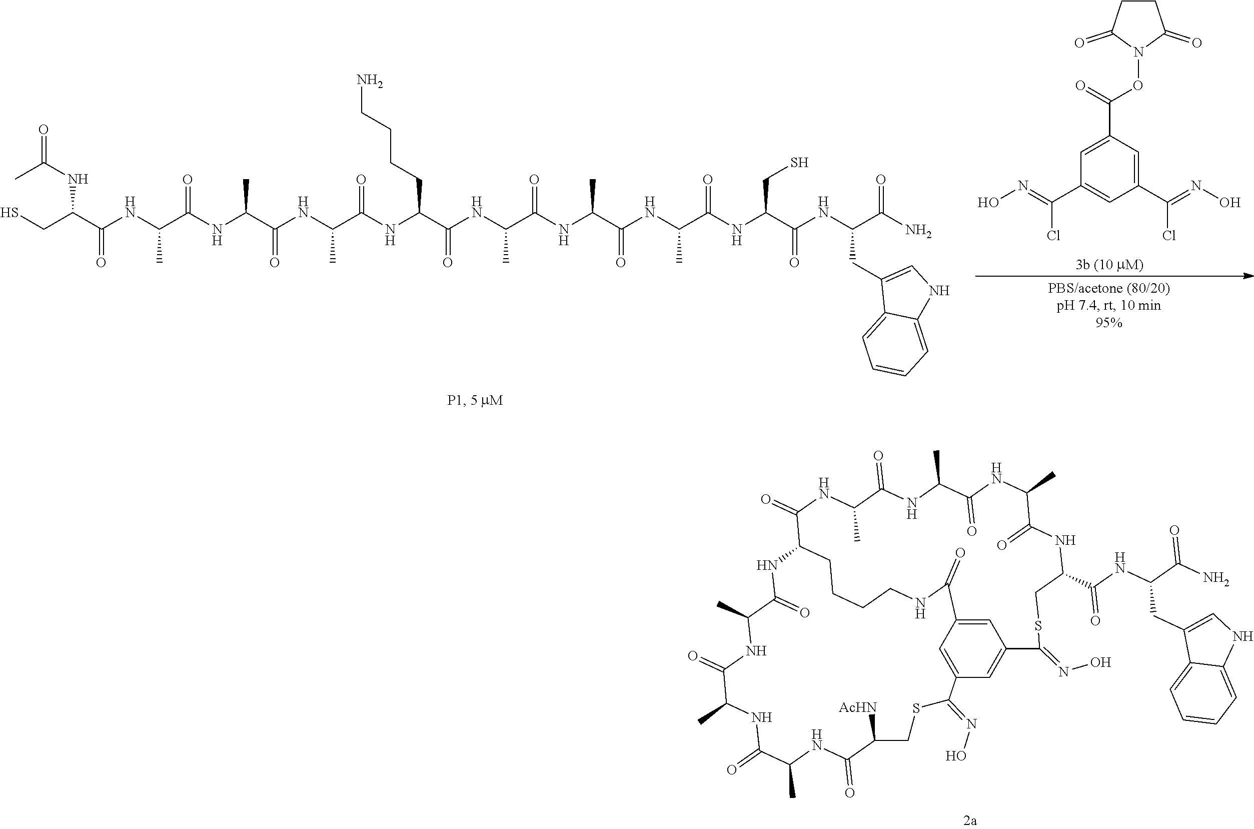 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](C)NC(=O)[C@H](CCCCNC3=O)NC(=O)[C@H](C)NC(=O)[C@H](C)NC(=O)[C@H](C)NC1=O]

4.1.2 Bicyclization of (Ac)CAKACW-NH₂ (SEQ ID NO: 6) with BC-OSu (3b); SEQ ID NOS 6 and 47, Respectively.

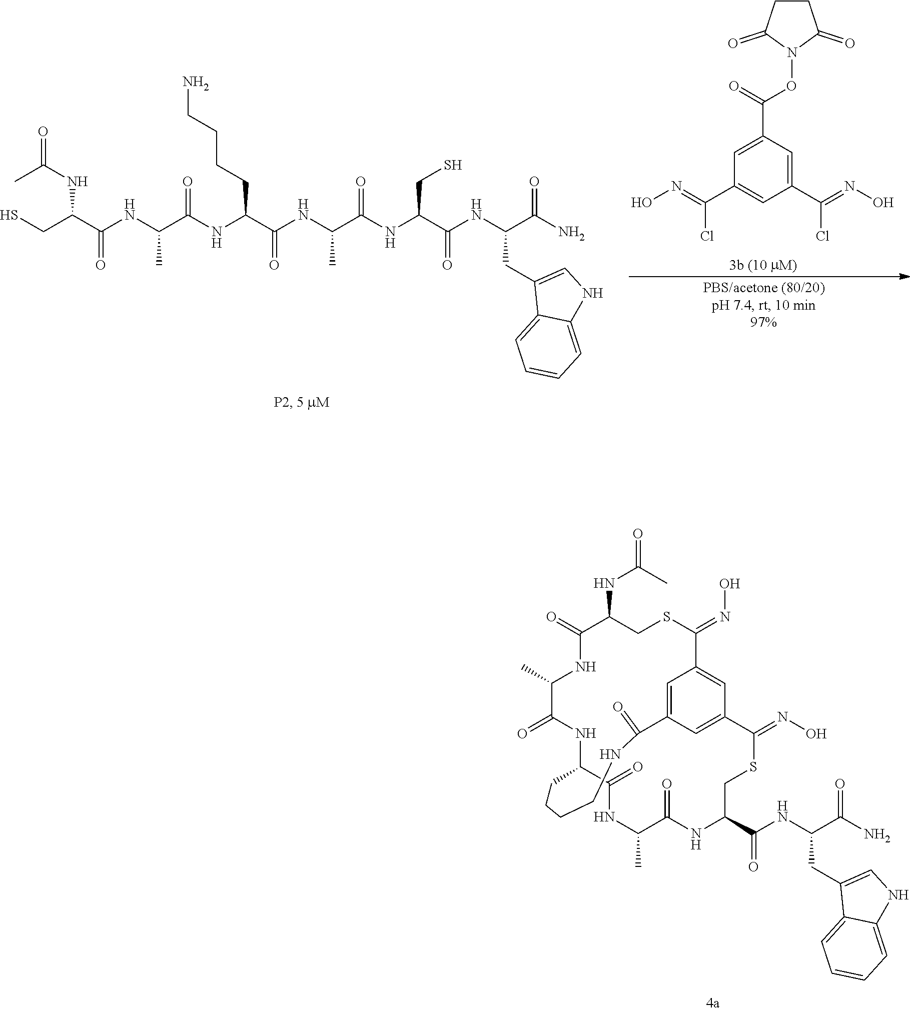 [SMILES: CC(=O)N[C@@H](CS)C(=O)N[C@@H](C)C(=O)N[C@@H](CCCCN)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](CCCCNC3=O)NC(=O)[C@H](C)NC1=O]

4.1.3 Bicyclization of (Ac)CYKSCW-NH₂ (SEQ ID NO: 7) with BC-TFP (3a); SEQ ID NOS 7 and 48, Respectively.

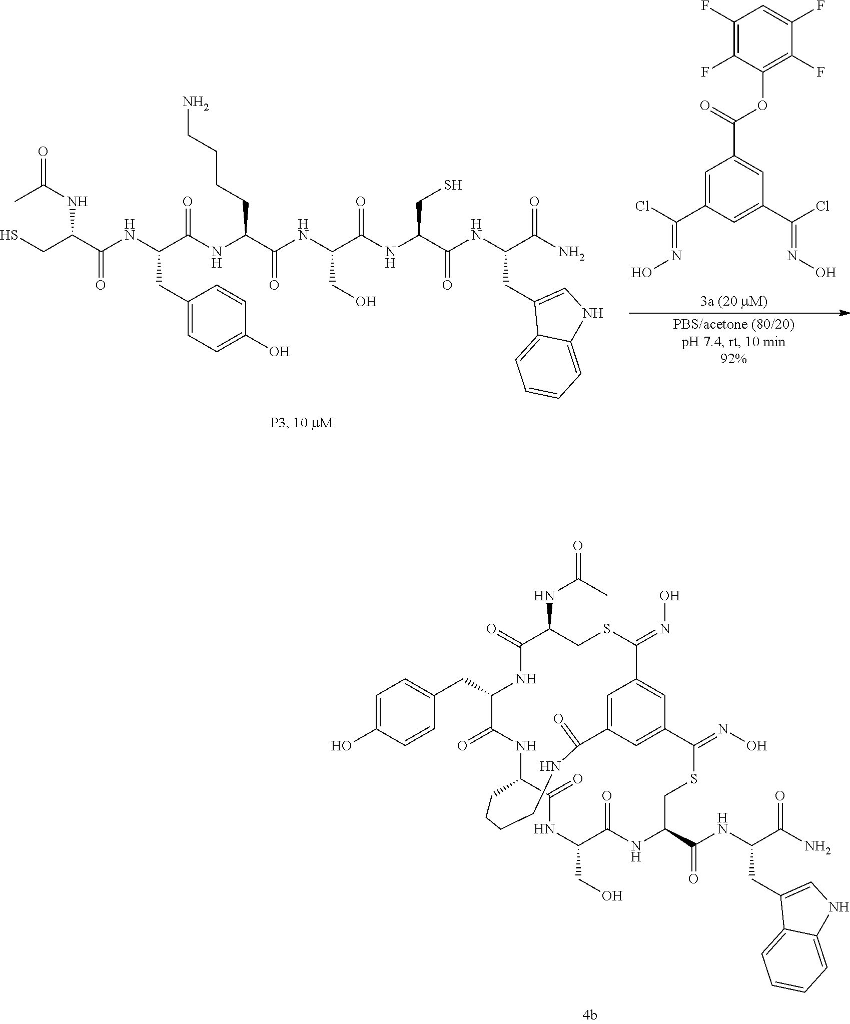 [SMILES: CC(=O)N[C@@H](CS)C(=O)N[C@@H](Cc1ccc(O)cc1)C(=O)N[C@@H](CCCCN)C(=O)N[C@@H](CO)C(=O)N[C@@H](CS)C(=O)N[C@@H](Cc1c[nH]c2ccccc12)C(N)=O ; O=C(Oc1c(F)c(F)cc(F)c1F)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](CO)NC(=O)[C@H](CCCCNC3=O)NC(=O)[C@H](Cc2ccc(O)cc2)NC1=O]

4.1.4 Bicyclization of (Ac)CAAAAKAAAACW-NH₂ (SEQ ID NO: 8) with BC-OSu (3b); SEQ ID NOS 8 and 49, Respectively.

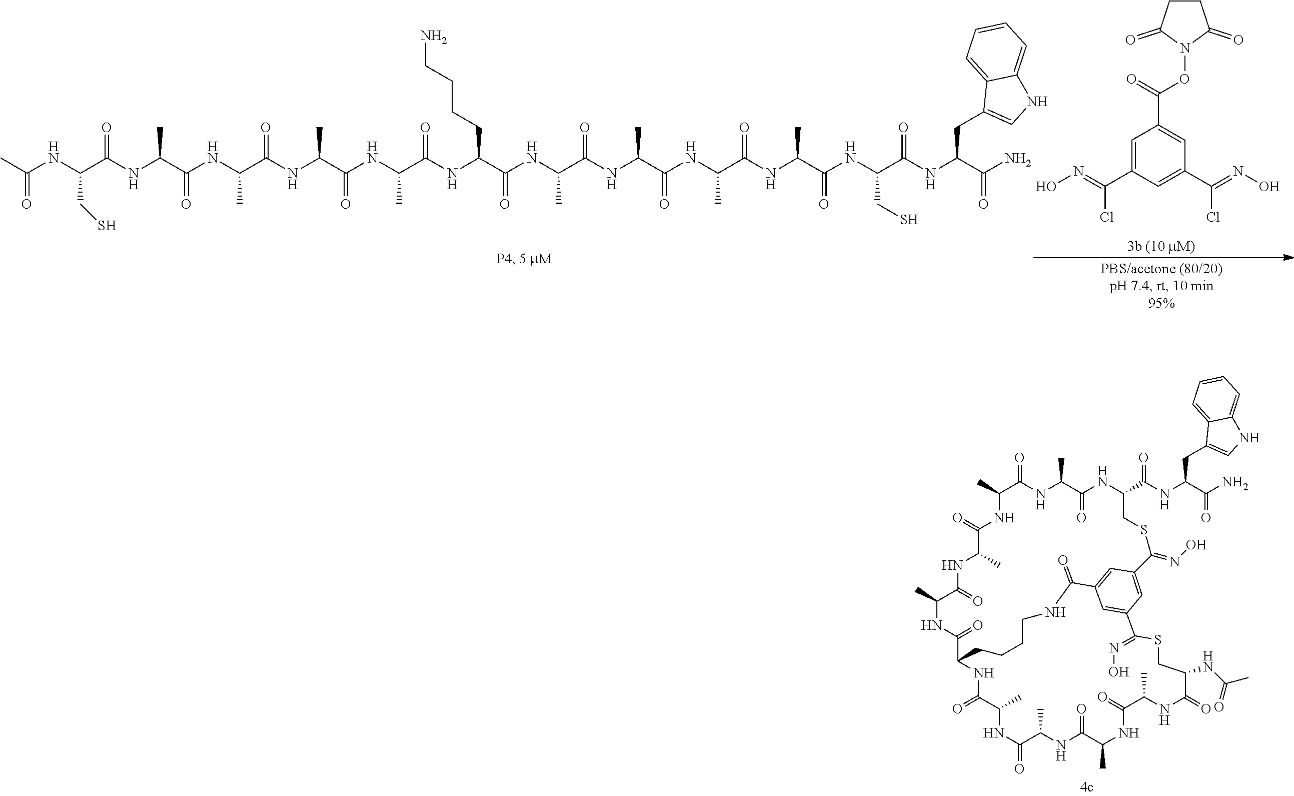 [SMILES: CC(=O)N[C@@H](CS)C(=O)N[C@@H](C)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](C)NC(=O)[C@H](C)NC(=O)[C@H](CCCCNC3=O)NC(=O)[C@H](C)NC(=O)[C@H](C)NC(=O)[C@H](C)NC(=O)[C@H](C)NC1=O]

4.1.5 Bicyclization of (Ac)CAEAKAEACW-NH₂ (SEQ ID NO: 9) with BC-TFP (3a); SEQ ID NOS 9 and 50, Respectively.

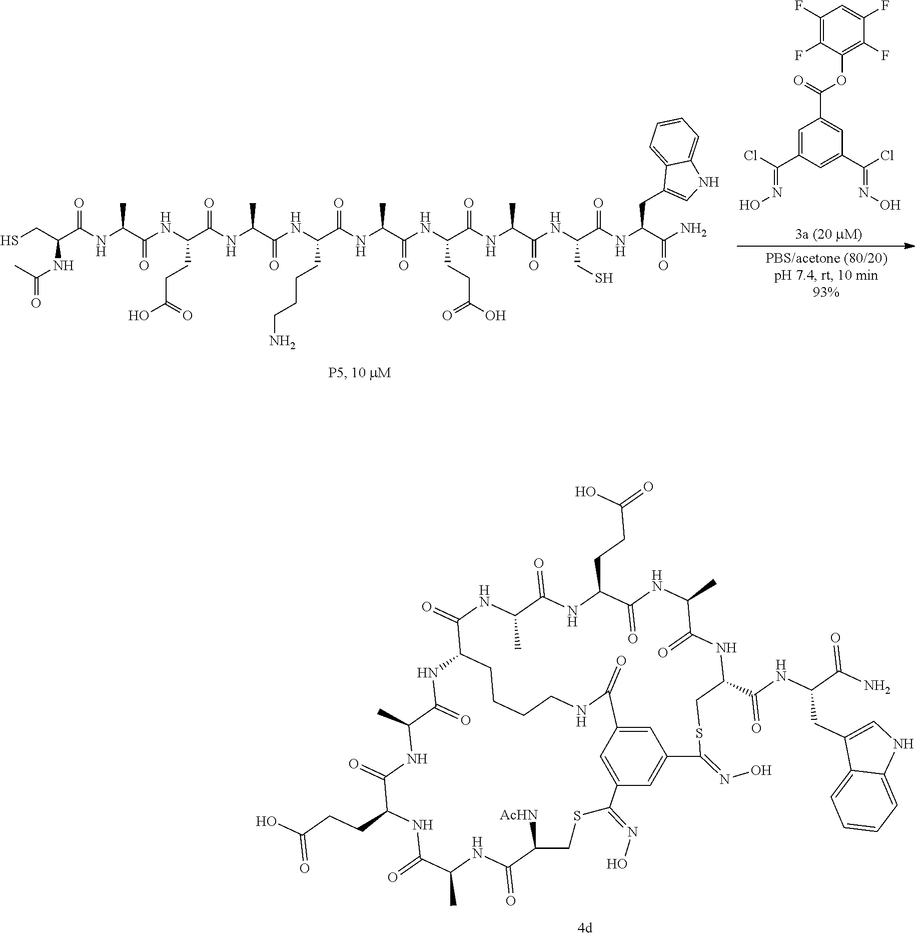 [SMILES: CC(=O)N[C@@H](CS)C(=O)N[C@@H](C)C(=O)N[C@@H](CCC(=O)O)C(=O)N[C@@H](C)C(=O)N[C@@H](CCCCN)C(=O)N[C@@H](C)C(=O)N[C@@H](CCC(=O)O)C(=O)N[C@@H](C)C(=O)N[C@@H](CS)C(=O)N[C@@H](Cc1c[nH]c2ccccc12)C(N)=O ; O=C(Oc1c(F)c(F)cc(F)c1F)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](CCC(=O)O)NC(=O)[C@H](C)NC(=O)[C@H](CCCCNC3=O)NC(=O)[C@H](C)NC(=O)[C@H](CCC(=O)O)NC(=O)[C@H](C)NC1=O]

4.1.6 Bicyclization of (Ac)CAHAKAHACW-NH₂ (SEQ ID NO: 10) with BC-OSu (3b); SEQ ID NOS 10 and 51, Respectively.

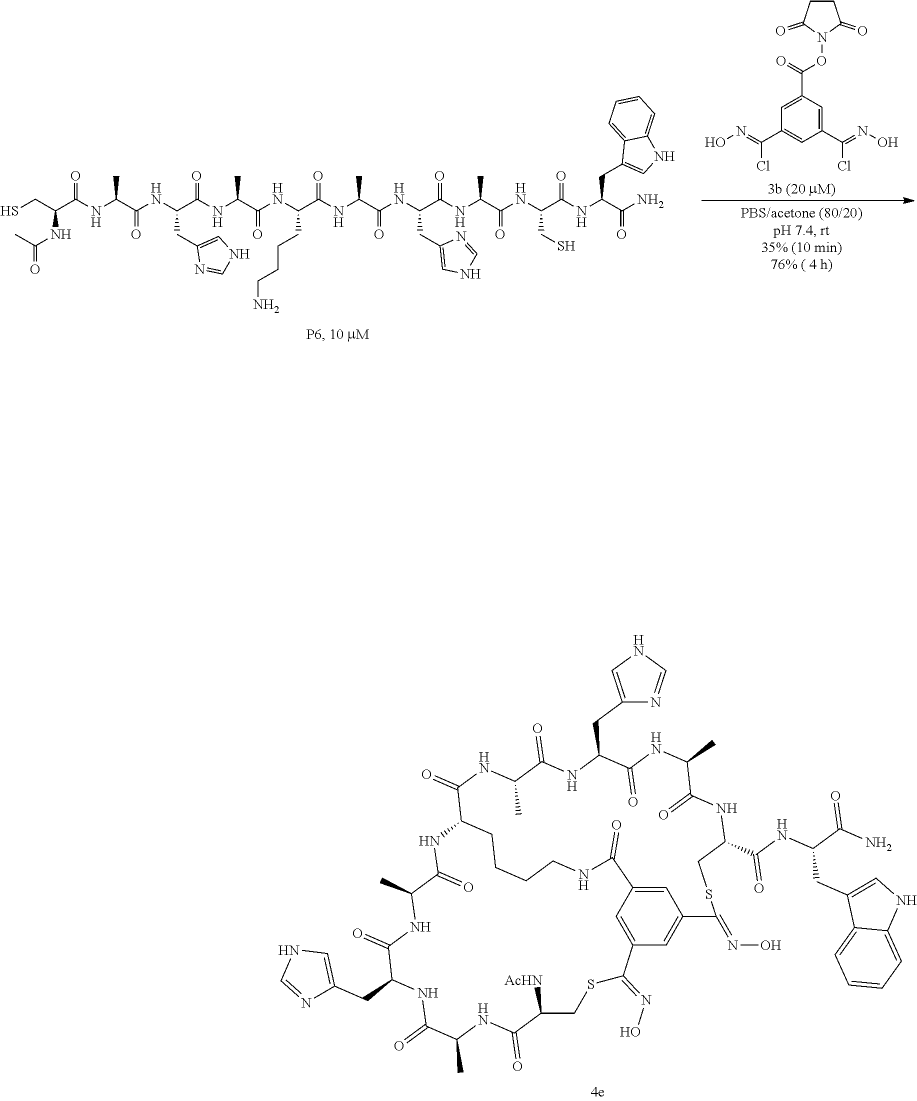 [SMILES: CC(=O)N[C@@H](CS)C(=O)N[C@@H](C)C(=O)N[C@@H](Cc1c[nH]cn1)C(=O)N[C@@H](C)C(=O)N[C@@H](CCCCN)C(=O)N[C@@H](C)C(=O)N[C@@H](Cc1c[nH]cn1)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](Cc2c[nH]cn2)NC(=O)[C@H](C)NC(=O)[C@H](CCCCNC3=O)NC(=O)[C@H](C)NC(=O)[C@H](Cc2c[nH]cn2)NC(=O)[C@H](C)NC1=O]

4.1.7 Bicyclization of (Ac)CAKAHQAACW-NH₂ (SEQ ID NO: 11) with BC-OSu (3b); SEQ ID NOS 11 and 52, Respectively.

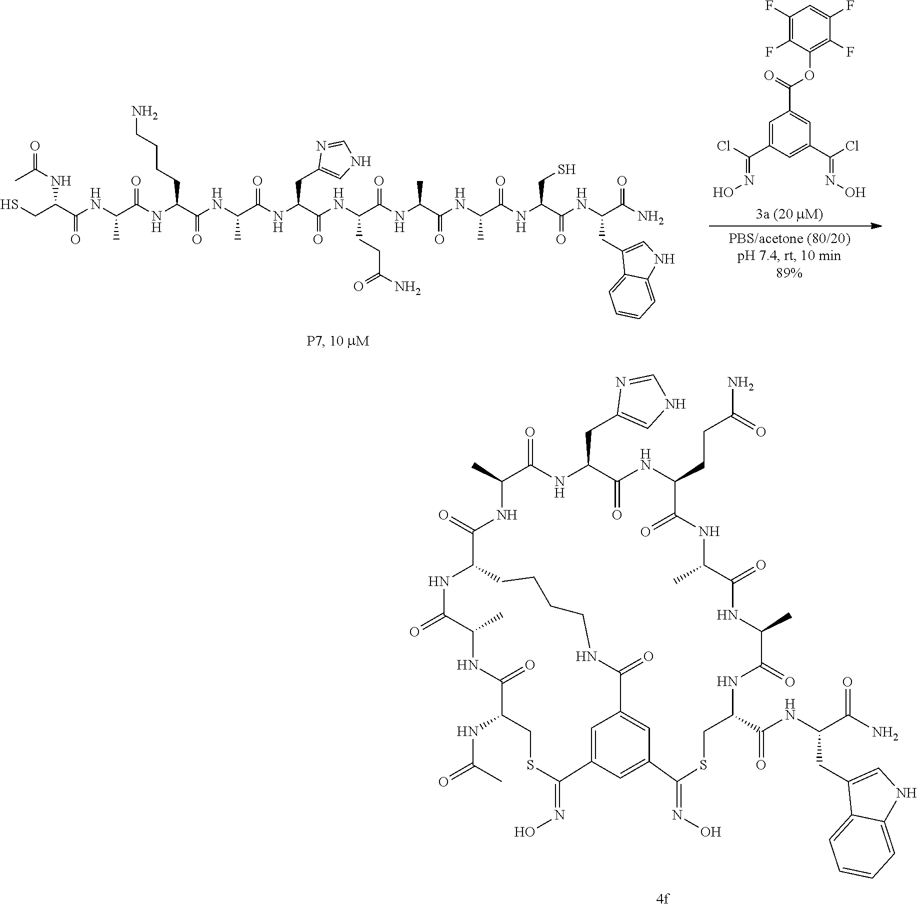 [SMILES: CC(=O)N[C@@H](CS)C(=O)N[C@@H](C)C(=O)N[C@@H](CCCCN)C(=O)N[C@@H](C)C(=O)N[C@@H](Cc1c[nH]cn1)C(=O)N[C@@H](CCC(N)=O)C(=O)N[C@@H](C)C(=O)N[C@@H](C)C(=O)N[C@@H](CS)C(=O)N[C@@H](Cc1c[nH]c2ccccc12)C(N)=O ; O=C(Oc1c(F)c(F)cc(F)c1F)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](CCC(N)=O)NC(=O)[C@H](Cc2c[nH]cn2)NC(=O)[C@H](C)NC(=O)[C@H](CCCCNC3=O)NC(=O)[C@H](C)NC1=O]

4.1.8 Bicyclization of (Ac)CKCW-NH₂ (SEQ ID NO: 12) with BC-OSu (3b); SEQ ID NOS 12 and 53, Respectively.

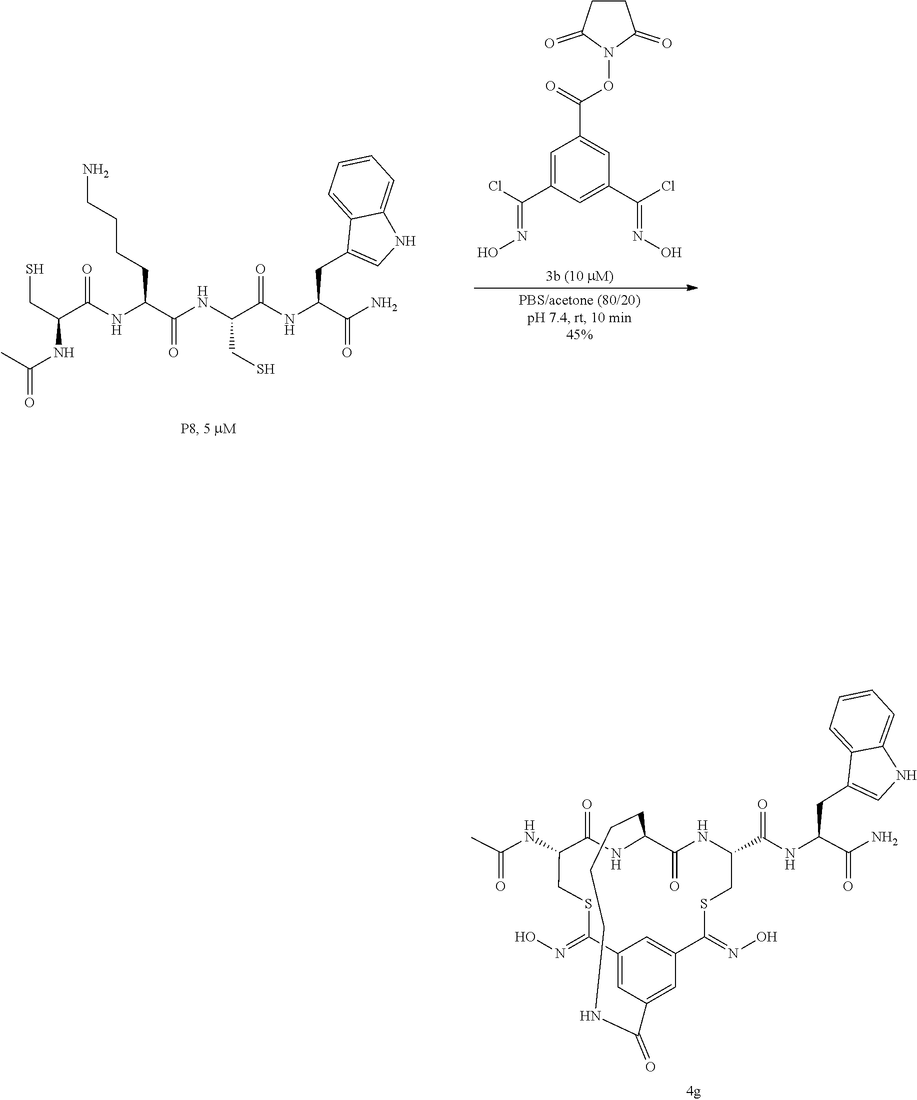 [SMILES: CC(=O)N[C@@H](CS)C(=O)N[C@@H](CCCCN)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](CCCCNC3=O)NC1=O]

Side products (SEQ ID NOS 98-99, respectively):

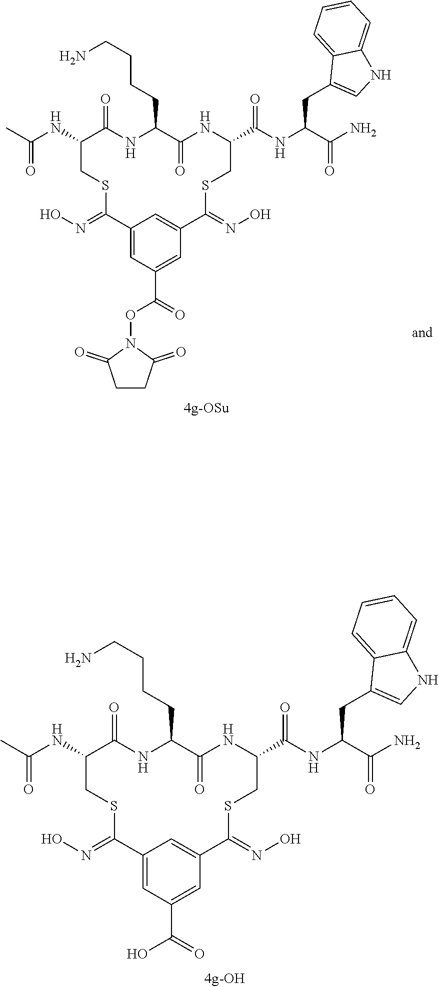 [SMILES: CC(=O)N[C@H]1CS/C(=N\O)c2cc(C(=O)ON3C(=O)CCC3=O)cc(c2)/C(=N/O)SC[C@@H](C(=O)N[C@@H](Cc2c[nH]c3ccccc23)C(N)=O)NC(=O)[C@H](CCCCN)NC1=O ; CC(=O)N[C@H]1CS/C(=N\O)c2cc(C(=O)O)cc(c2)/C(=N/O)SC[C@@H](C(=O)N[C@@H](Cc2c[nH]c3ccccc23)C(N)=O)NC(=O)[C@H](CCCCN)NC1=O]

0.9 Bicyclization of (Ac)KACEFCW-NH₂ (SEQ ID NO: 13) with BC-TFP); SEQ ID NOS 13 and 54, Respectively.

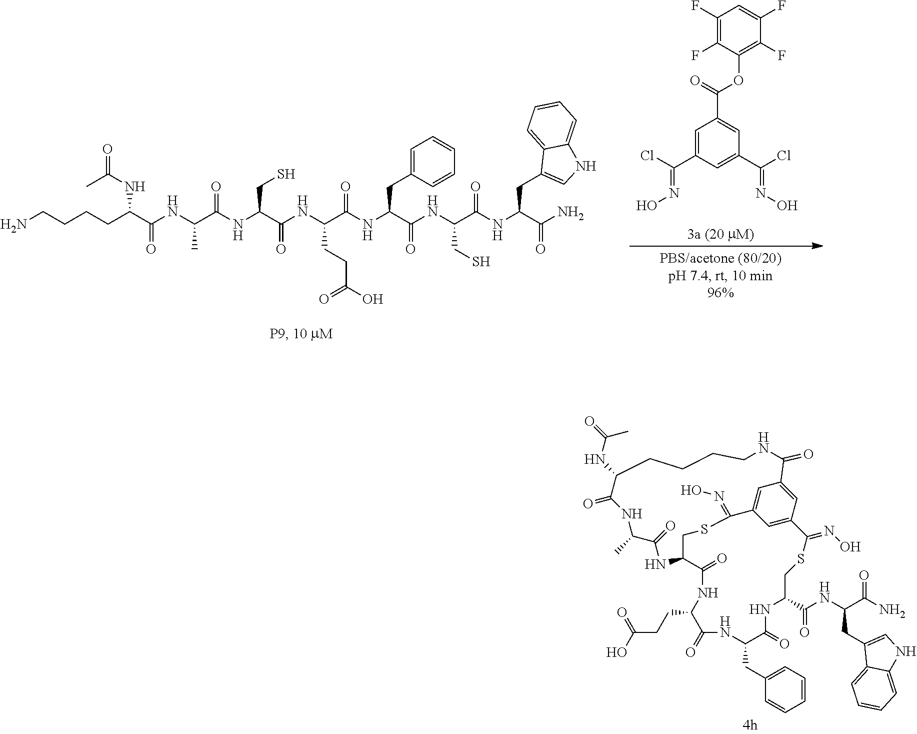 [SMILES: CC(=O)N[C@@H](CCCCN)C(=O)N[C@@H](C)C(=O)N[C@@H](CS)C(=O)N[C@@H](CCC(=O)O)C(=O)N[C@@H](Cc1ccccc1)C(=O)N[C@@H](CS)C(=O)N[C@@H](Cc1c[nH]c2ccccc12)C(N)=O ; O=C(Oc1c(F)c(F)cc(F)c1F)c1cc(/C(Cl)=N/O)cc(/C(Cl)=N/O)c1 ; CC(=O)N[C@@H]1CCCCNC(=O)c2cc3cc(c2)/C(=N/O)SC[C@H](C(=O)N[C@H](Cc2c[nH]c4ccccc24)C(N)=O)NC(=O)[C@H](Cc2ccccc2)NC(=O)[C@H](CCC(=O)O)NC(=O)[C@H](CS/C3=N\O)NC(=O)[C@H](C)NC1=O]

### 4.2 Peptide Bicyclization Through N-Terminus-Cys-Cys Stapling Using BC-TFP (3a) or BC-OSu (3b)

General procedure: 20 μL of bischlorooxime crosslinker 3a or 3b (100 μM stock solution in acetone) and 80 μL of N-terminus/cysteine-containing peptide (12.5 μM freshly made solution in PBS pH 7.4) were added to a 200 μL vial, resulting 10 μM of peptide and 20 μM of crosslinker. The mixture was pipetted several times and incubated at room temperature for 10 minutes. The reaction mixture was analyzed by LC-MS (Method A).

4.2.1 Bicyclization of GLIGCPFPASWC-NH₂ (SEQ ID NO: 14) with BC-OSu (3b); SEQ ID NOS 14 and 55, Respectively.

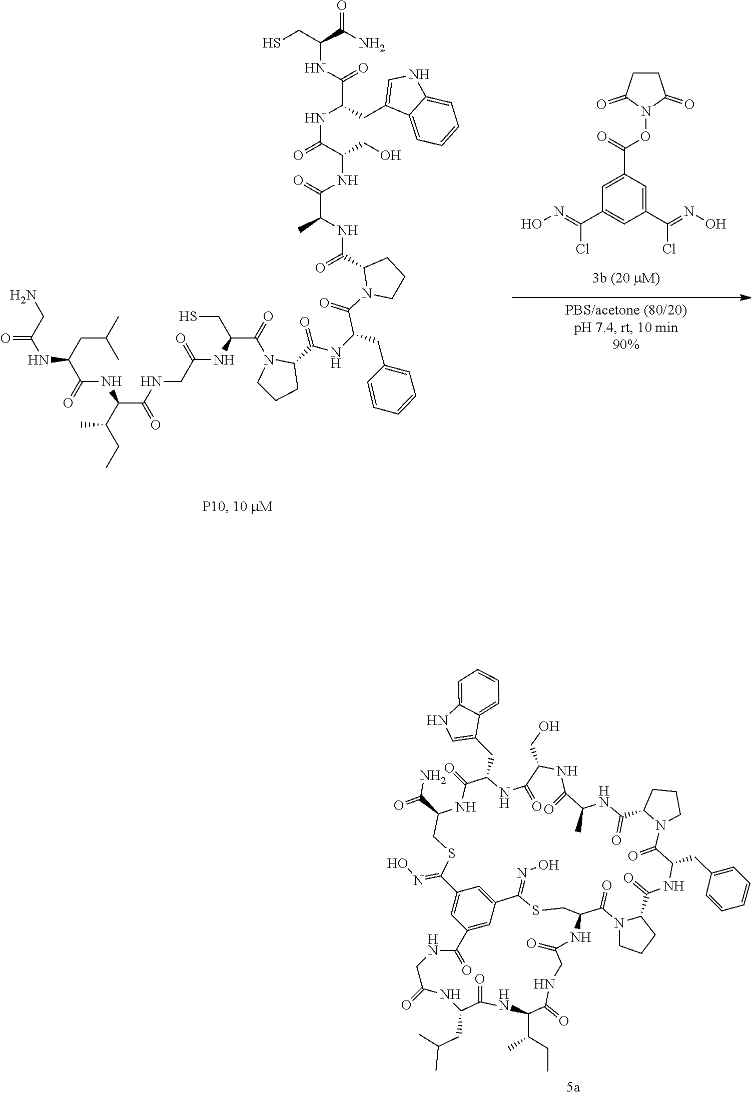 [SMILES: CC[C@H](C)[C@@H](NC(=O)[C@H](CC(C)C)NC(=O)CN)C(=O)NCC(=O)N[C@@H](CS)C(=O)N1CCC[C@H]1C(=O)N[C@@H](Cc1ccccc1)C(=O)N1CCC[C@H]1C(=O)N[C@@H](C)C(=O)N[C@@H](CO)C(=O)N[C@@H](Cc1c[nH]c2ccccc12)C(=O)N[C@@H](CS)C(N)=O ; O=C(ON1C(=O)CCC1=O)c1cc(/C(Cl)=N/O)cc(/C(Cl)=N/O)c1 ; CC[C@H](C)[C@H]1NC(=O)[C@H](CC(C)C)NC(=O)CNC(=O)c2cc3cc(c2)/C(=N/O)SC[C@H](NC(=O)CNC1=O)C(=O)N1CCC[C@H]1C(=O)N[C@@H](Cc1ccccc1)C(=O)N1CCC[C@H]1C(=O)N[C@@H](C)C(=O)N[C@@H](CO)C(=O)N[C@@H](Cc1c[nH]c2ccccc12)C(=O)N[C@H](C(N)=O)CS/C3=N\O]

4.2.2 Bicyclization of ALIGCPFPASWC-NH₂ (SEQ ID NO: 15) with BC-OSu (3b); SEQ ID NOS 15 and 56, Respectively.

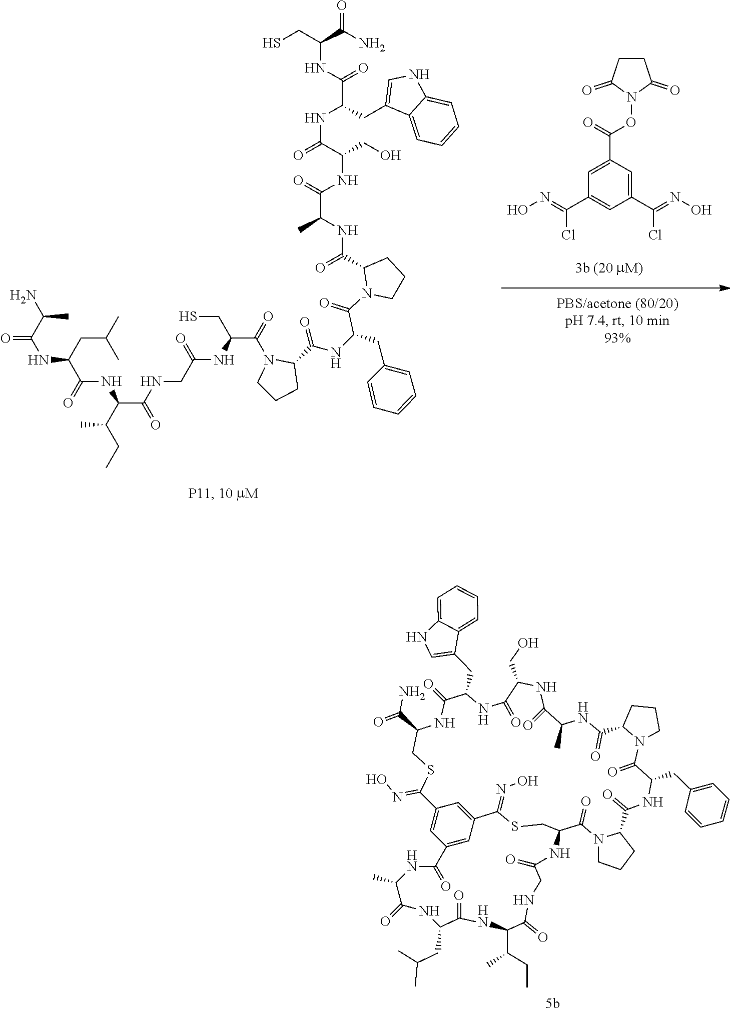

4.2.3 Bicyclization of ACPFPASWC-NH₂ (SEQ ID NO: 16) with BC-OSu (3b); SEQ ID NOS 16 and 57, Respectively.

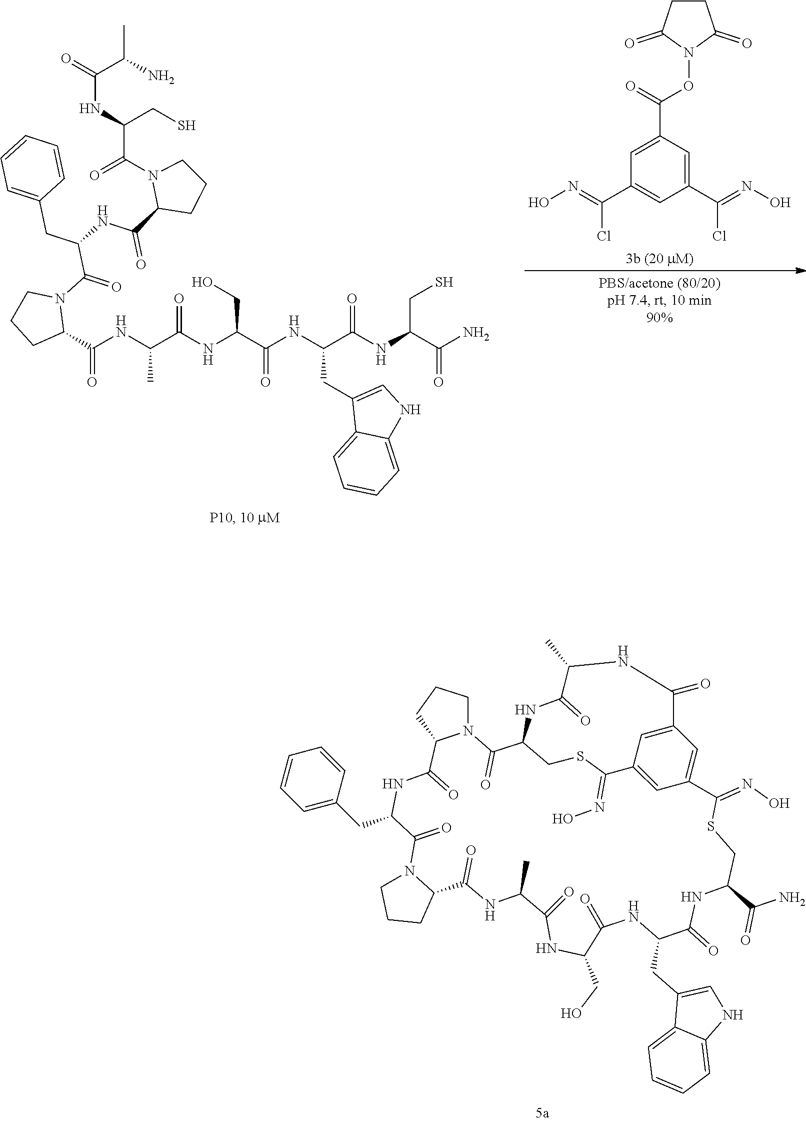 [SMILES: C[C@H](N)C(=O)N[C@@H](CS)C(=O)N1CCC[C@H]1C(=O)N[C@@H](Cc1ccccc1)C(=O)N1CCC[C@H]1C(=O)N[C@@H](C)C(=O)N[C@@H](CO)C(=O)N[C@@H](Cc1c[nH]c2ccccc12)C(=O)N[C@@H](CS)C(N)=O ; O=C(ON1C(=O)CCC1=O)c1cc(/C(Cl)=N/O)cc(/C(Cl)=N/O)c1 ; C[C@@H]1NC(=O)c2cc3cc(c2)/C(=N/O)SC[C@H](NC1=O)C(=O)N1CCC[C@H]1C(=O)N[C@@H](Cc1ccccc1)C(=O)N1CCC[C@H]1C(=O)N[C@@H](C)C(=O)N[C@@H](CO)C(=O)N[C@@H](Cc1c[nH]c2ccccc12)C(=O)N[C@H](C(N)=O)CS/C3=N\O]

4.2.4 Bicyclization of ACLIPTWGGC-NH₂ (SEQ ID NO: 17) with BC-TFP (3a); SEQ ID NOS 17 and 58, Respectively.

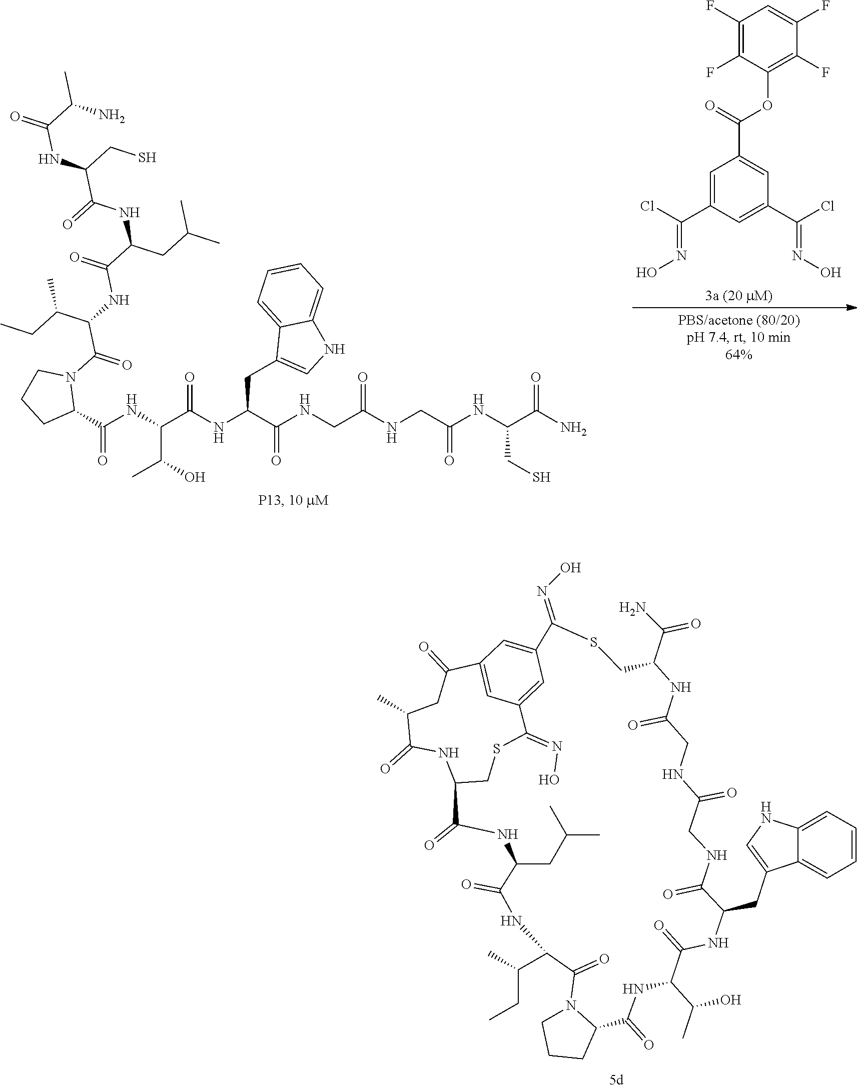 [SMILES: CC[C@H](C)[C@H](NC(=O)[C@H](CC(C)C)NC(=O)[C@H](CS)NC(=O)[C@H](C)N)C(=O)N1CCC[C@H]1C(=O)N[C@H](C(=O)N[C@@H](Cc1c[nH]c2ccccc12)C(=O)NCC(=O)NCC(=O)N[C@@H](CS)C(N)=O)[C@@H](C)O ; O=C(Oc1c(F)c(F)cc(F)c1F)c1cc(/C(Cl)=N/O)cc(/C(Cl)=N/O)c1 ; CC[C@H](C)[C@@H]1NC(=O)[C@H](CC(C)C)NC(=O)[C@@H]2CS/C(=N\O)c3cc(cc(c3)/C(=N/O)SC[C@H](C(N)=O)NC(=O)CNC(=O)CNC(=O)[C@@H](Cc3c[nH]c4ccccc34)NC(=O)[C@H]([C@@H](C)O)NC(=O)[C@@H]3CCCN3C1=O)C(=O)C[C@@H](C)C(=O)N2]

4.2.5 Bicyclization of PCPFPASWC-NH₂ (SEQ ID NO: 18) with BC-TFP (3a); SEQ ID NOS 18 and 59, Respectively.

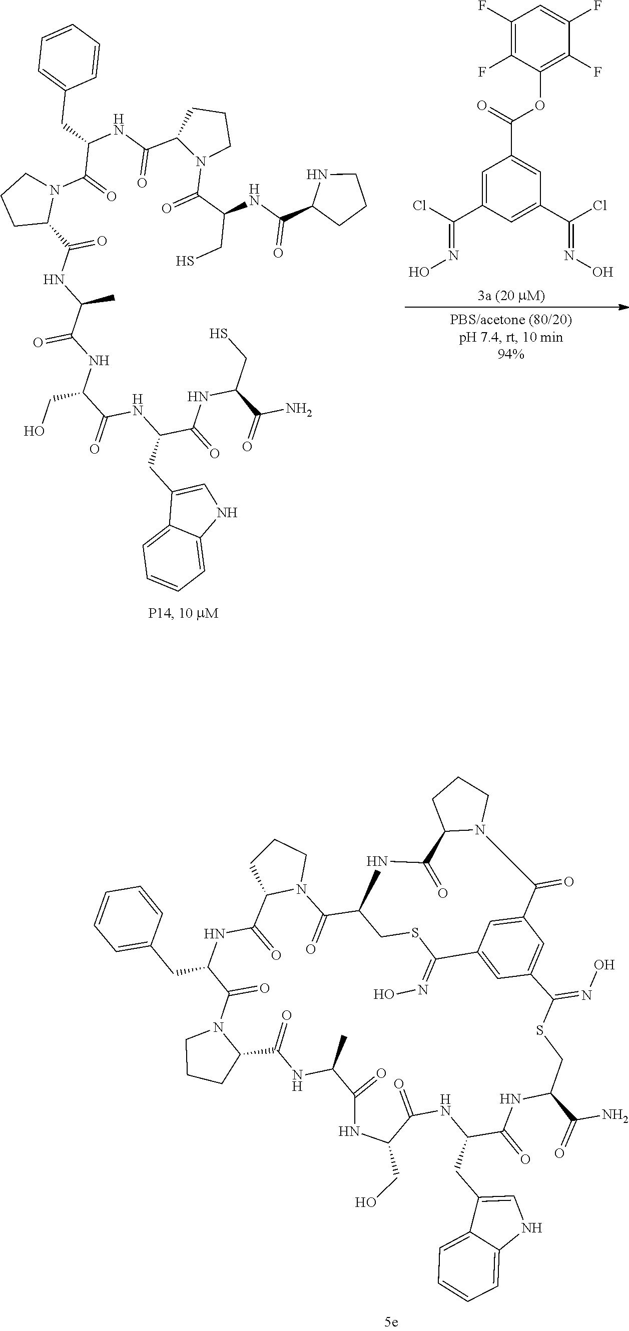 [SMILES: C[C@H](NC(=O)[C@@H]1CCCN1C(=O)[C@H](Cc1ccccc1)NC(=O)[C@@H]1CCCN1C(=O)[C@H](CS)NC(=O)[C@@H]1CCCN1)C(=O)N[C@@H](CO)C(=O)N[C@@H](Cc1c[nH]c2ccccc12)C(=O)N[C@@H](CS)C(N)=O ; O=C(Oc1c(F)c(F)cc(F)c1F)c1cc(/C(Cl)=N/O)cc(/C(Cl)=N/O)c1 ; C[C@@H]1NC(=O)[C@@H]2CCCN2C(=O)[C@H](Cc2ccccc2)NC(=O)[C@@H]2CCCN2C(=O)[C@@H]2CS/C(=N\O)c3cc(cc(c3)/C(=N\O)SC[C@@H](C(N)=O)NC(=O)[C@H](Cc3c[nH]c4ccccc34)NC(=O)[C@H](CO)NC1=O)C(=O)N1CCC[C@@H]1C(=O)N2]

4.2.6 Bicyclization of ACFMQEPLYICG-NH₂ (SEQ ID NO: 19) with BC-TFP (3a); SEQ ID NOS 19 and 60, Respectively.

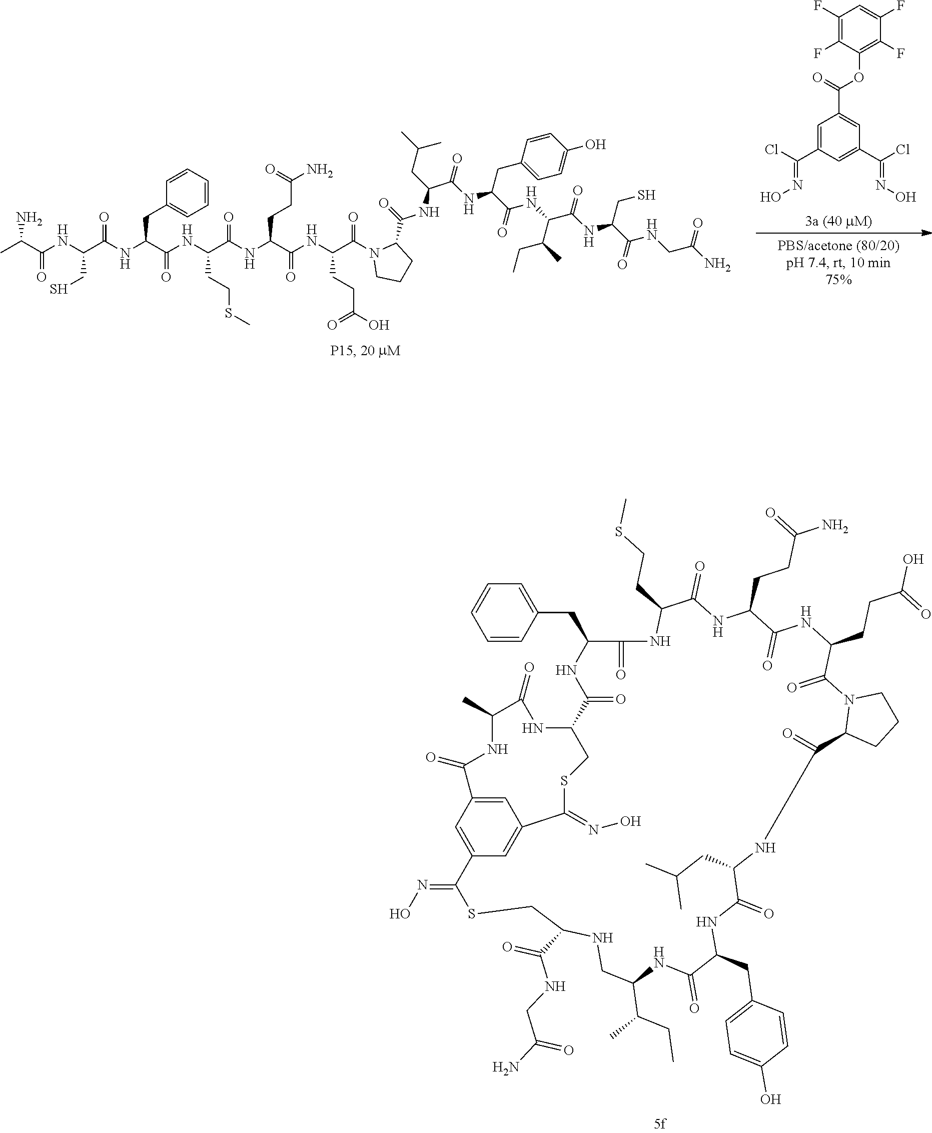

4.2.7 Bicyclization of ALIGCESAYKNTAQCW-NH₂ (SEQ ID NO: 20) with 3b (SEQ ID NOS 20, 100, and 61, Respectively).

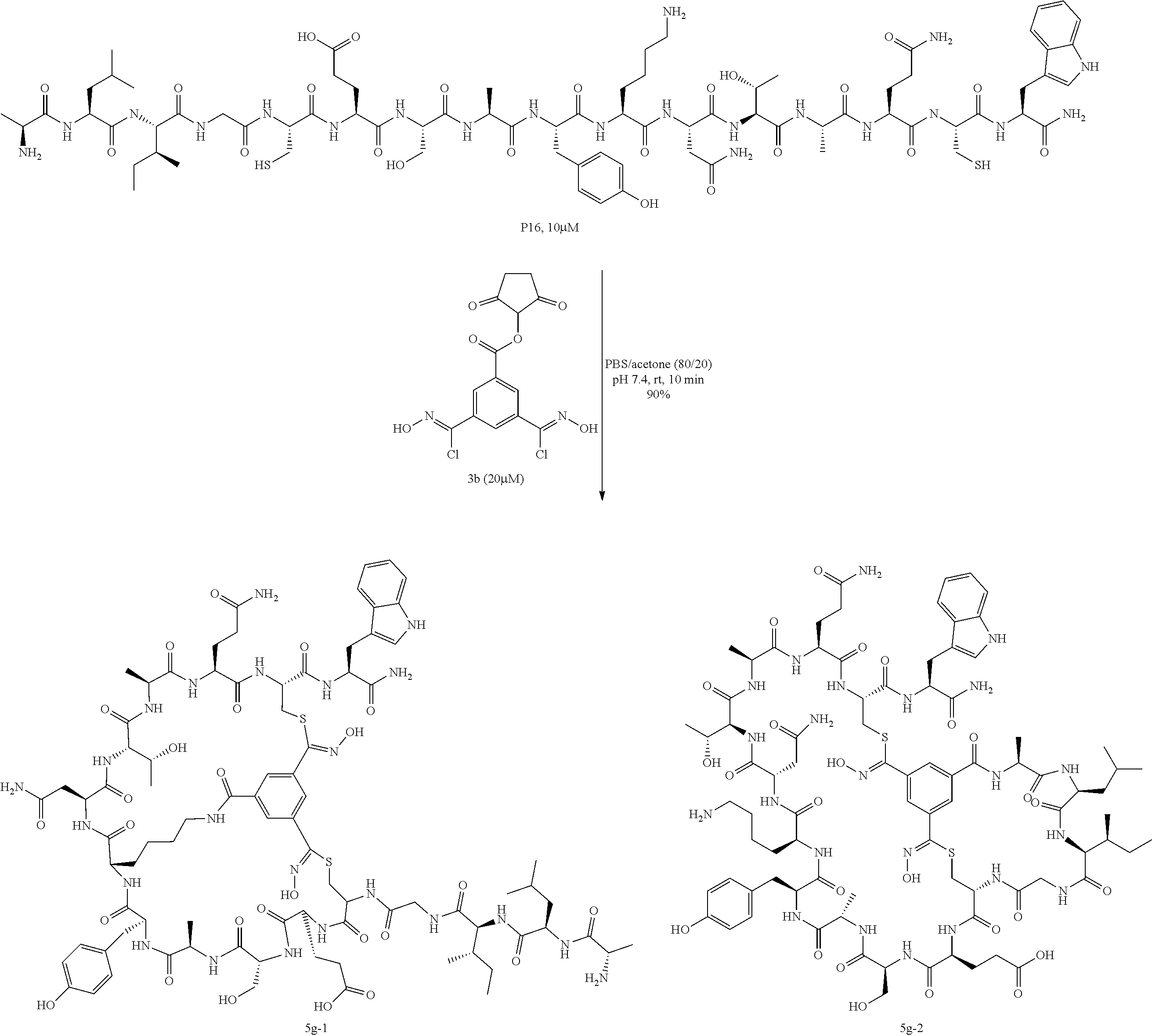

4.2.8 Bicyclization of ALIGCPFPARWC-NH₂ (SEQ ID NO: 22) with BC-OSu (3b); SEQ ID NOS 22, 62, 22, and 62, Respectively.

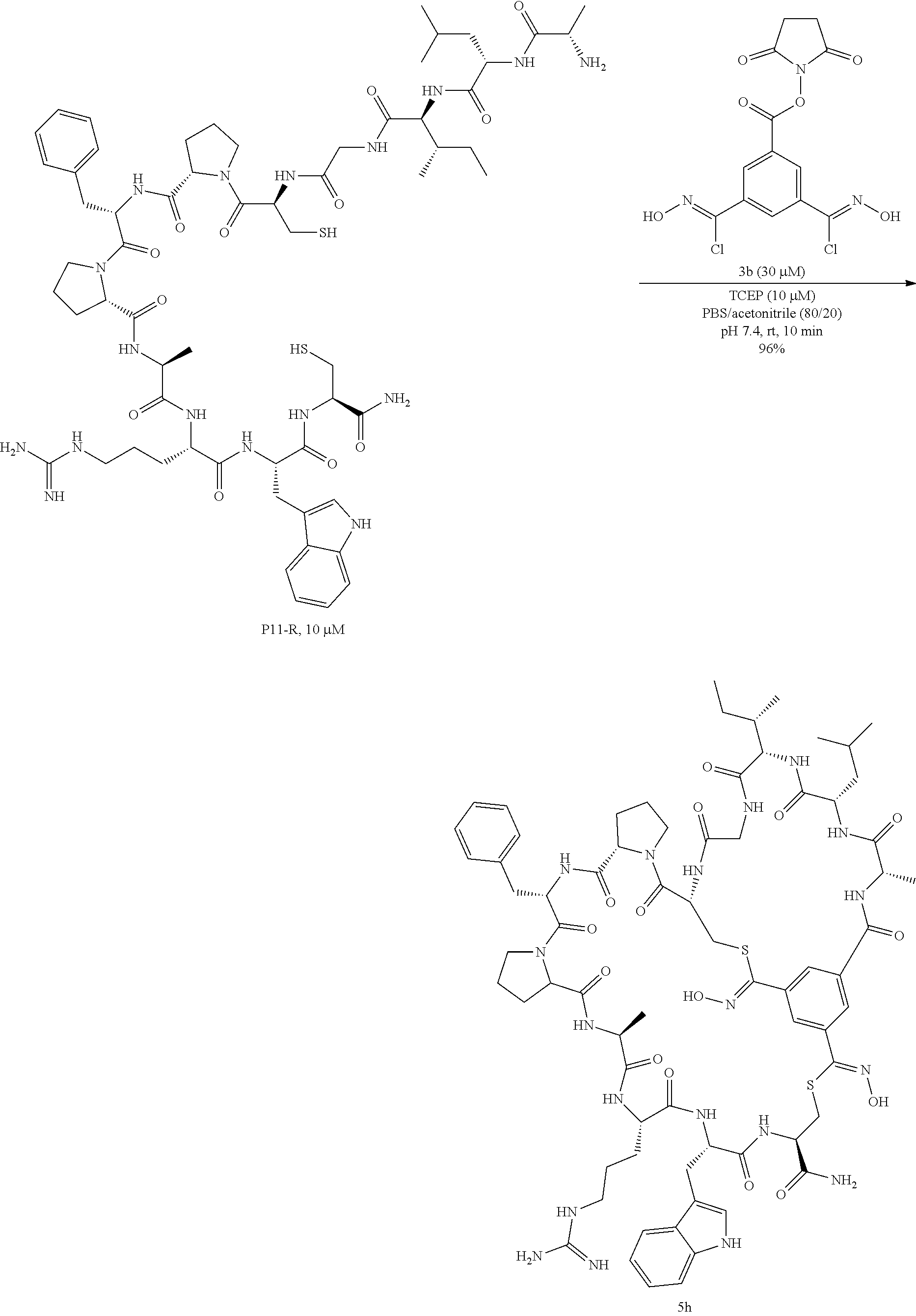

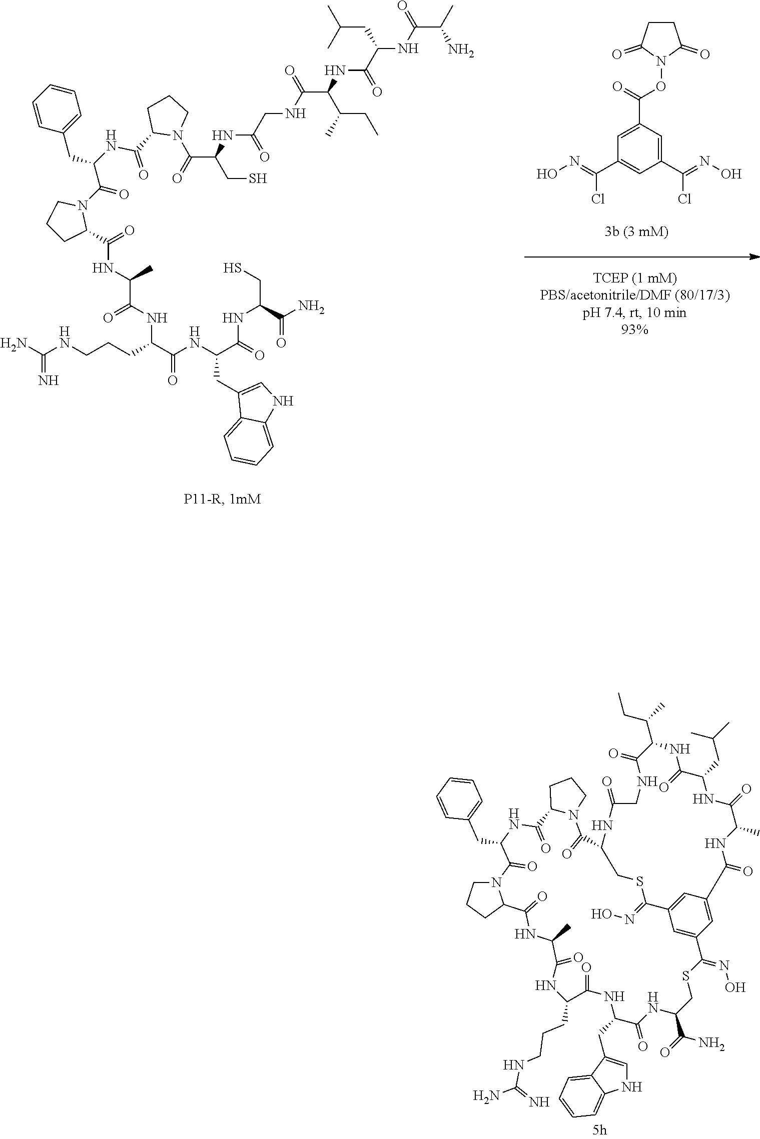

For 1 mM scale reaction: To a 5-mL Eppendorf tube in a total volume of 2000 μL, 60 μL of the BC-OSu crosslinker 3b (100 mM stock solution in DMF), 1596 μL of P11-R peptide (1.25 mM freshly made solution in PBS pH 7.4), 4.0 μL of TCEP (0.5 M stock solution in water), and 340 μL acetonitrile was vortexed several times. The reaction was incubated at room temperature for 10 minutes. The reaction mixture was analyzed by LC-MS (Method C).

### 4.3 Disulfide Reduction/Bicyclization of N-Terminal Proline-containing Peptide (SEQ ID NOS 63, 18, and 59, Respectively)

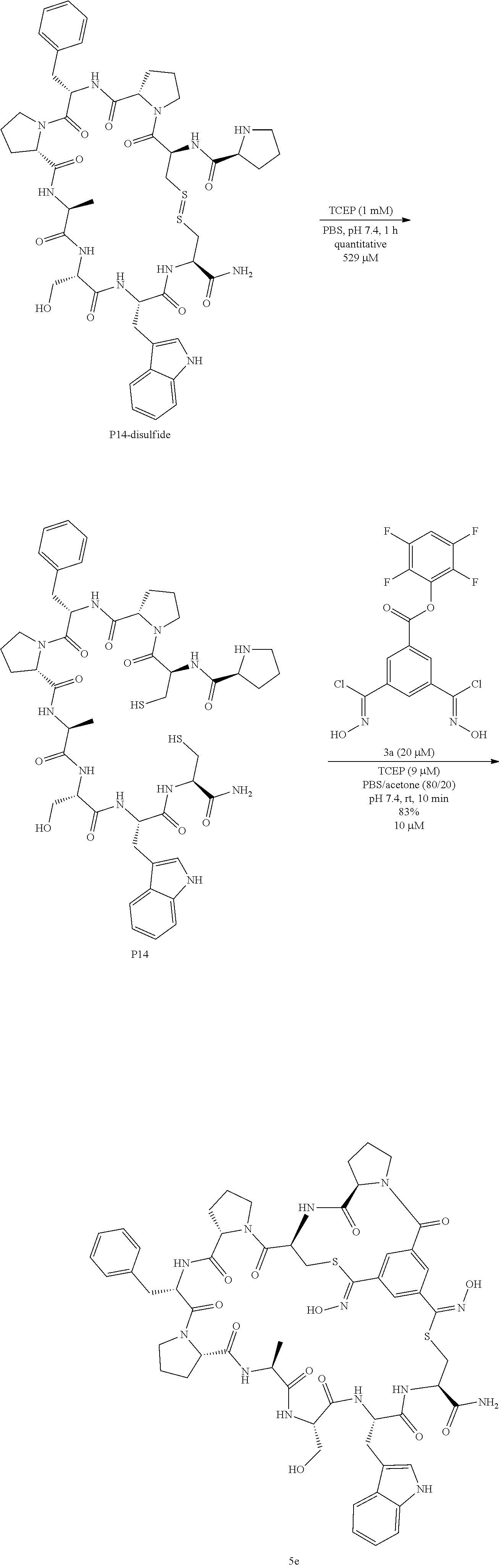

200 μL of disulfide peptide P14-disulfide (529 μM stock solution in PBS, pH 7.4) was reduced with 1 mM TCEP at room temperature for 1 hour to give peptide P14. Aliquot 1.9 μL of the peptide P14/TCEP mixture and diluted it to 80 μL with PBS (pH 7.4), and then mixed with 20 μL of BC-TFP (3a) (100 μM stock solution in acetone), resulting 10 μM of peptide, 9 μM of TCEP and 20 μM of 3a. The mixture was pipetted several times and incubated at room temperature for 10 minutes. The reaction mixture was analyzed by LC-MS (Method A).

### 4.4 Solvent Compatibility of Bicyclization Protocols (SEQ ID NOS 5 and 42, Respectively)

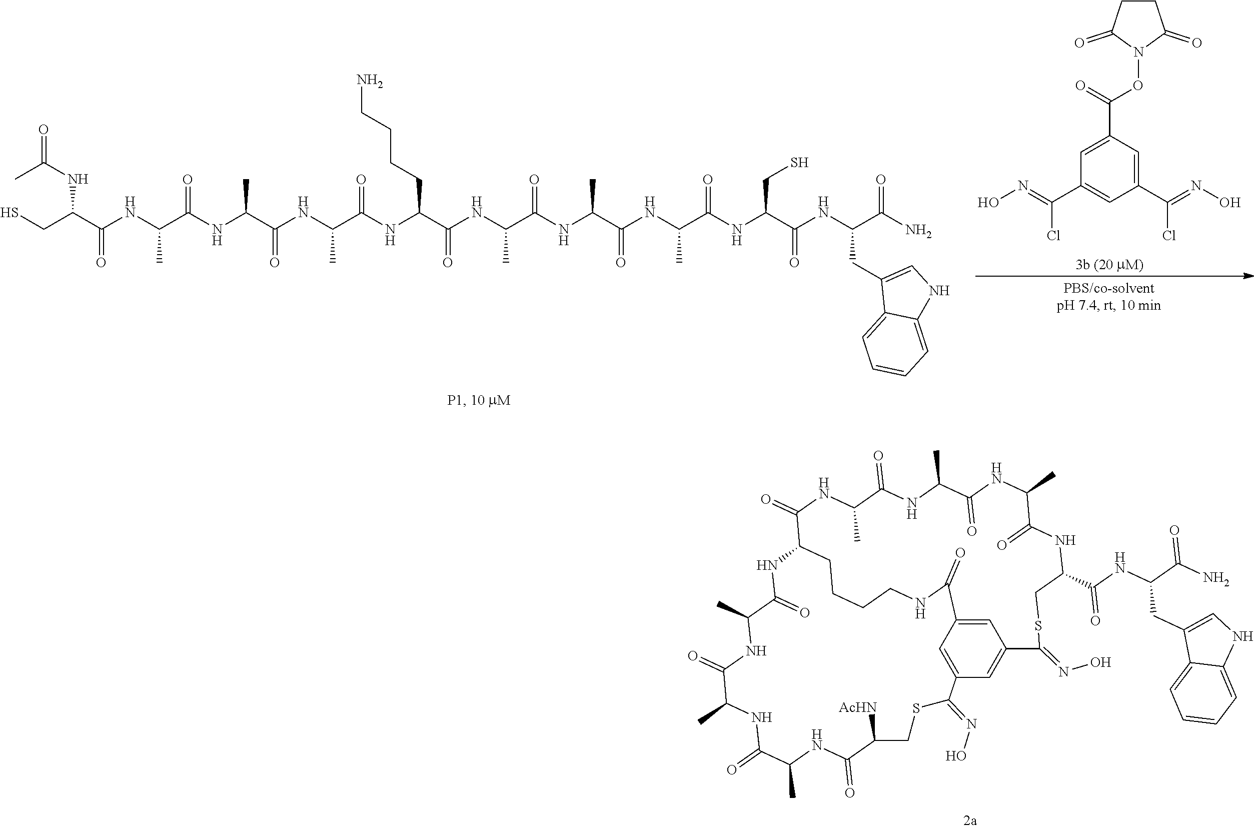 [SMILES: CC(=O)N[C@@H](CS)C(=O)N[C@@H](C)C(=O)N[C@@H](C)C(=O)N[C@@H](C)C(=O)N[C@@H](CCCCN)C(=O)N[C@@H](C)C(=O)N[C@@H](C)C(=O)N[C@@H](C)C(=O)N[C@@H](CS)C(=O)N[C@@H](Cc1c[nH]c2ccccc12)C(N)=O ; O=C(ON1C(=O)CCC1=O)c1cc(/C(Cl)=N/O)cc(/C(Cl)=N/O)c1 ; CC(=O)N[C@H]1CS/C(=N\O)c2cc3cc(c2)/C(=N/O)SC[C@@H](C(=O)N[C@@H](Cc2c[nH]c4ccccc24)C(N)=O)NC(=O)[C@H](C)NC(=O)[C@H](C)NC(=O)[C@H](C)NC(=O)[C@H](CCCCNC3=O)NC(=O)[C@H](C)NC(=O)[C@H](C)NC(=O)[C@H](C)NC1=O]

### General Procedure:

Bischlorooxime crosslinker 3b (1 or 2 mM stock solution in corresponding co-solvent) and peptide P1 (freshly made solution in PBS pH 7.4) were mixed in PBS/co-solvent (100 μL) in a 200 μL vial, resulting 10 μM of peptide and 20 μM of crosslinker. The mixture was pipetted several times and incubated at room temperature for 10 minutes. The reaction mixture was analyzed by LC-MS (Method C).

### 4.5 Stability Studies of Bicyclic Peptide in Aqueous Solution (SEQ ID NO: 62)

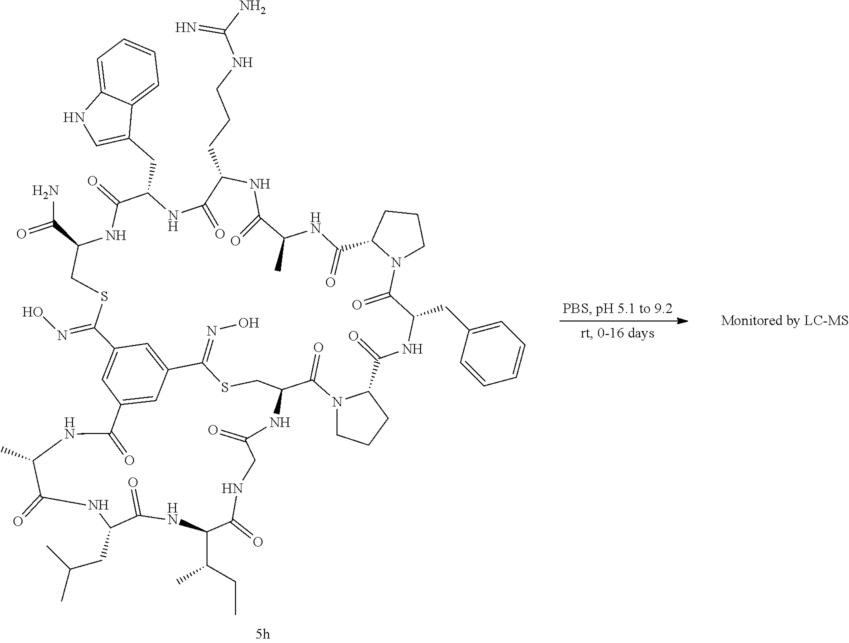 [SMILES: CC[C@H](C)[C@H]1NC(=O)[C@H](CC(C)C)NC(=O)[C@H](C)NC(=O)c2cc3cc(c2)/C(=N/O)SC[C@H](NC(=O)CNC1=O)C(=O)N1CCC[C@H]1C(=O)N[C@@H](Cc1ccccc1)C(=O)N1CCC[C@H]1C(=O)N[C@@H](C)C(=O)N[C@@H](CCCNC(=N)N)C(=O)N[C@@H](Cc1c[nH]c2ccccc12)C(=O)N[C@H](C(N)=O)CS/C3=N\O]

The bicyclic peptide 5h was prepared according to above mentioned procedure and purified by RP-HPLC. The solution of bicyclic peptide 5h (200 μM) in PBS was incubated at pH 5.1, pH 7.4, and pH 9.2, respectively. The sample was analyzed by LC-MS at different time points over 16 days (Method C). The bicyclic peptide 5h remained intact after 16 days incubation at pH tested. Hydrolysis or decomposition of 5h was not observed, revealing good stability of the bicyclic peptide in aqueous solution.

### Example 5

### Bicyclization of Fusion SSL11 Protein Using BC-OSu (3b)

### 5.1 Engineered SSL11 Expression and Purification

The plasmid containing the SSL11 gene was constructed using standard cloning techniques. The NEB genomic DNA extraction kit (NEB #T3010) was used to extract the DNA from the S. aureus strain D592 following the manufacturer's protocol. The SSL11 gene was amplified from the genomic DNA by polymerase chain reaction (PCR) using Primer 1 and Primer 2 (Genewiz, Boston, MA) with the incorporated restriction enzyme sites, KpnI and NotI, respectively. The resulting amplified SSL11 gene was subsequently cloned into a pET-22b vector using the two restriction enzyme sites. The sequence of the plasmid was confirmed by Sanger sequencing.

Further cloning was performed to insert a double stranded duplex containing the TEV cleavage site and the CAAAAKAAAAC peptide (SEQ ID NO: 23) at the C-terminal of the SSL11 protein. First, a NcoI restriction enzyme site was inserted at the C-terminal of the plasmid containing the SSL11 protein between the NotI site and the His-tag, using the NEB Q5 mutagenesis kit (NEB #E0554) with Primer 3 and Primer 4, following the manufacturer's protocol. A 92 base pair double stranded duplex was ordered from IDT that contained NotI and NcoI restriction enzyme sites, the TEV cleavage site, and the CAAAAKAAAAC peptide (SEQ ID NO: 23). In parallel, the mutated plasmid and the double stranded duplex were digested with the enzymes NotI and NcoI. The double stranded duplex was ligated into the C-terminal of the SSL11 plasmid and transformed into Top10 E. coli cells. Finally, the C-terminal His-tag was mutated to the N-terminal by two subsequent mutagenesis using the NEB Q5 mutagenesis kit. First, the C-terminal His-tag was deleted using Primer 5 and Primer 6, and then the His-tag was inserted into the N-terminal using Primer 7 and Primer 8. The cloned plasmid was sequenced to confirm the inserted double stranded duplex and the His-tag at the N-terminal.

The final plasmid was transformed into BL21 (DE3) E. coli cells and then these cells were used for protein expression. A single colony from a plate was incubated overnight at 37° C. in 10 mL LB broth containing 100 μg/mL ampicillin. The 10 mL overnight culture was inoculated in 1 L of LB broth in a 4 L flask and grown to an OD₆₀₀ 0.4-0.6 at 37° C. while shaking at 250 rpm. Then the culture was induced with IPTG (1 mM final concentration) to induce the overexpression of the SSL11 protein. The culture was incubated overnight for 16 hours at room temperature. The cells were harvested by centrifugation (5000 g, 20 minutes, 4° C.) and lysed in lysis buffer (20 mM Tris, 300 mM NaCl, 10 mM imidazole) by sonication on ice. The lysed cells were centrifuged (6500 g, 20 minutes, 4° C.) again to remove the insoluble cell debris and the supernatant was purified using Ni-NTA resin. The resin was washed 4 times with 1 ml of the washing buffer (20 mM Tris, 300 mM NaCl, 25 mM imidazole) and then the protein was eluted from the resin using 2.5 ml of elution buffer twice (20 mM Tris, 300 mM NaCl, 250 mM imidazole).

The purified protein was desalted using a PD-10 desalting column (GE healthcare) and stored in PBS buffer pH 7.4 at 4° C. for further use.

Primer 1:
(SEQ ID NO: 24)
5′-CCGGGTACCAGTACATTAGAGGTTAGATCA-3′
Primer 2:
(SEQ ID NO: 25)
5′-CGGGCGGCCGCTAAATTCACTTCAATTTT-3′
Primer 3:
(SEQ ID NO: 26)
5′-CCATGGAGAGCACCACCACCACCAC-3′
Primer 4:
(SEQ ID NO: 27)
5′-GCTGCGGCGAGTGCGGCCGCTAAATTC-3′
Primer 5:
(SEQ ID NO: 28)
5′-TGAGATCCGGCTGCTAACAAAG-3′
Primer 6:
(SEQ ID NO: 29)
5′-GCCACCACCCCAACATGC-3′
Primer 7:
(SEQ ID NO: 30)
5′-CACCACCACGGCCTGAACGATATTTTTG-3′
Primer 8:
(SEQ ID NO: 31)
5′-ATGATGATGCATCGGTAATTGTTCCTC-3′
Double-stranded duplex is shown in FIG. 39.

The protein sequence is shown below, and the final construct is illustrated in FIG. 40.

(SEQ ID NO: 32)
MHHHHHHGLNDIFEAQKIEWHEGTSTLEVRSQATQDLSEYYKGRGFELT
NVTGYKYGNKVTFIDNSQQIDVTLTGNEKLTVKDDDEVSNVDVFVVREG
SDKSAITTSIGGITKTNGTQHKDTVQNVNLSVSKSTGQHTTSVTSEYYS
IYKEEISLKELDFKLRKHLIDKHDLYKTEPKDSKIRITMKNGGYYTFEL
NKKLQPHRMGDTIDSRNIEKIEVNLAAAENLYFQGGGCAAAAKAAAACW
GGG*

### 5.2 Modification of Fusion SSL11 Protein Using 3b

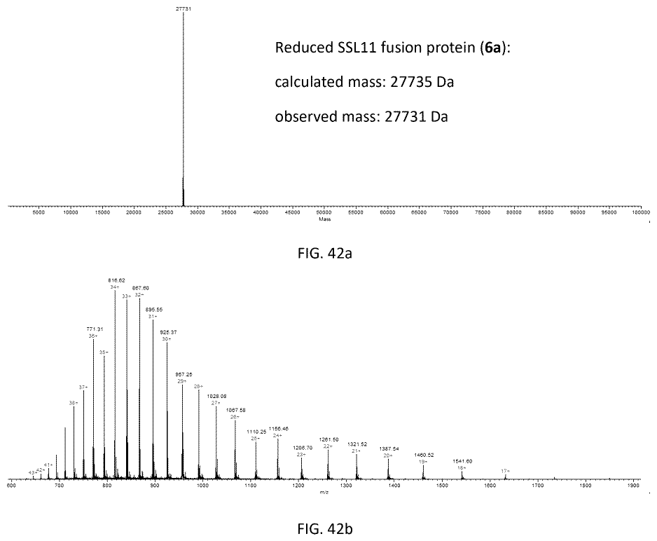

SSL11fusion protein (61 μM in PBS, pH 7.4) was reduced with 1 mM TCEP in PBS buffer (pH 7.4) at 4° C. overnight to give reduced SSL11 protein 6a (FIGS. 42a-42b). 131 μL of reduced SSL11 protein 6a (61 μM in PBS with 16 equivalents of TCEP, pH 7.4) was diluted with 53 μM of PBS (pH 7.4), and mixed with 16 μL of BC-OSu (3b) (1 mM stock solution in acetone), resulting 40 μM of protein and 80 μM of crosslinker in 8% acetone/PBS buffer (Note: 20% of acetone in PBS would lead to the precipitation of protein from the solution). The mixture was pipetted several times, incubated at room temperature, and analyzed with LC-MS.

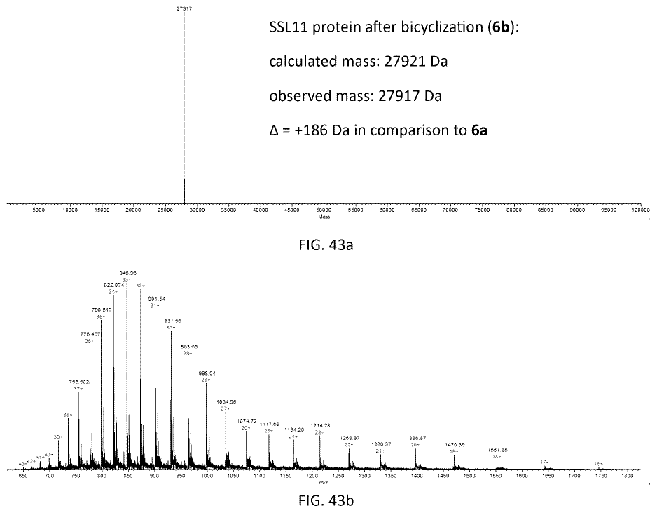

After 15 min incubation, a full conversion of 6a was observed. The molecular weight of protein increased by 186 Da after modification, which is consistent with the mass change for bicyclization reaction (calculated mass change Δ=+186 Da). This result clearly indicates that the protein reacts with 1 equivalent of crosslinker 3b to form a bicyclic SSL11 protein 6b as the observed product (FIGS. 43a-43b).

### 5.3 Enzymatic Fragmentation of Bicyclic SSL11 Protein (6b) Using TEV Protease

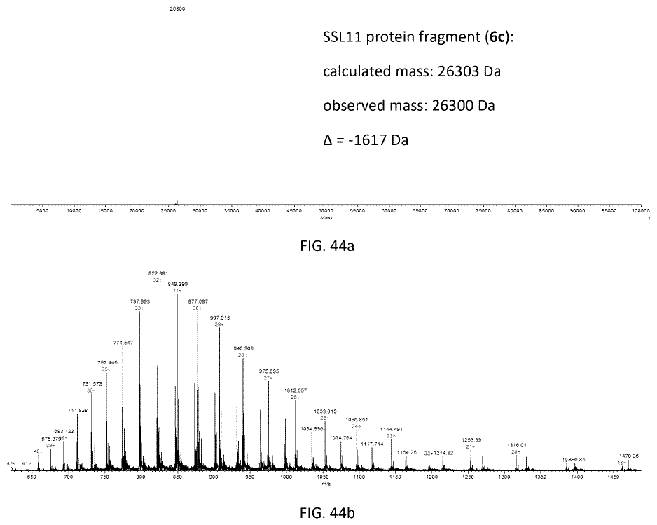

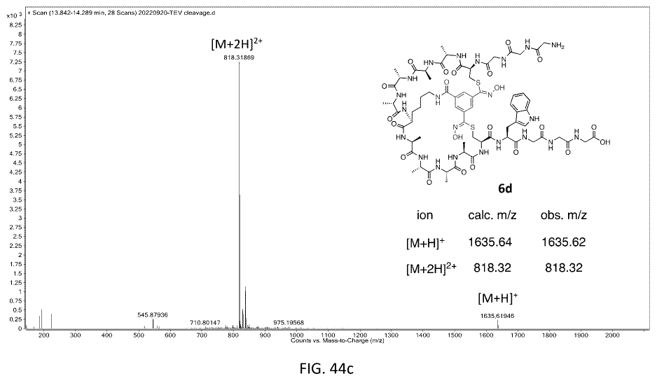

50 μL of bicyclic SSL11 protein (6b) (40 μM in PBS, pH 7.4) was treated with 15 μL of TEV protease (3 μM in stock solution in PBS, pH 7.4), resulting 36 μM of protein and 0.8 μM of TEV protease. The mixture was incubated at room temperature overnight and the fragment was analyzed with LC-MS. The observed mass belongs to the unmodified protein fragment, implying that the bicyclization happened only at the peptide fragment (FIGS. 44a-44c).

### 5.4 Modification of Fusion SSL11 Protein Using 3a

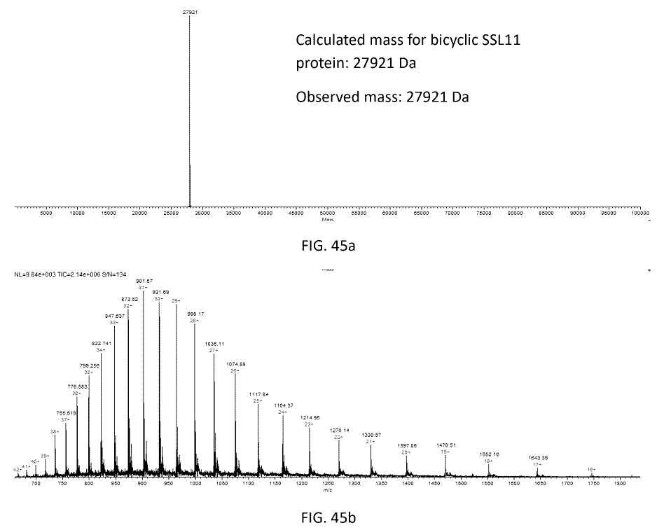

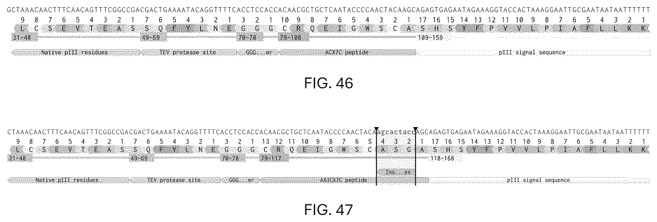

37.8 μL of reduced SSL11 protein 6a (39.7 μM in PBS, pH 7.4) was diluted with 2.2 μL of PBS (pH 7.4) and mixed with 3.0 μL of BC-OTFP (3a) (1 mM stock solution in acetone) and 7.0 μL of acetone, resulting 30 μM of protein and 60 μM of crosslinker in 20% acetone/PBS buffer. The mixture was pipetted several times, incubated at room temperature, and analyzed with LC-MS. After 25 min incubation, full conversion of reduced SSL11 protein 6a to bicyclic product was observed (FIGS. 45a-45b).

### Example 6

### Bicyclization of N-Terminal Peptide on Phage Using BC-OSu (3b)

### 6.1 Construction of Phage Variants

A discreet phage variant containing an N-terminal ACX₇C (ACSWGIEQRC (SEQ ID NO: 1)) peptide with a TEV protease cleavage site (ENLYFQ'S, “ENLYFQ” disclosed as SEQ ID NO: 33) between the pill protein and peptide was isolated. To amplify the ACX₇C phage variant, 50 mL of LB was added to a 250-mL Erlenmeyer flask with 500 μL of overnight E. coli strain ER2738 (NEB #E4104) and 10 μL of phage and incubated overnight at 37° C. while shaking at 250 rpm. The next day the cells were pelleted by centrifugation (7830 rpm, 20 minutes, 4° C.) and the phage (in supernatant) was poured into a new tube containing ⅙ total volume of 20% polyethylene glycol-8000 (PEG)/2.5 M NaCl. The precipitating phage was left at 4° C. overnight and then centrifuged (7830 rpm, 20 minutes, 4° C.). The pelleted phage was resuspended in ˜5 mL PBS buffer pH 7.4 to make a phage solution of ˜10¹² pfu/mL and stored at 4° C. for further use.

Another phage variant was constructed to include three amino acids between the N-terminus alanine and cysteine, AX₃CX₇C (AGSACSWGIEQRC (SEQ ID NO: 2)). The plasmid from the ACX₇C phage was mutated using the NEB Q5 mutagenesis kit (NEB #E0554) with Primer 9 and Primer 10, following the manufacturer's protocol. 5 μL of the mutated plasmid was transformed into NEB 5-alpha Competent E. coli cells (NEB, #C2987H) by heat shock and then grown for 1 hour in 950 μL of SOC outgrowth medium at 37° C., shaking at 250 rpm. The transformed cells were diluted (10¹, 102, 103) and mixed with 200 μL of ER2738 cells (OD₆₀₀˜0.5) and 790 μL of top agar and then plated on IPTG/XgaI plates. The plates were incubated overnight at 37° C. to allow for growth of the phage on the plates, which were then sequenced to confirm the mutation. To amplify the AX₃CX₇C phage variant, 50 mL of LB was added to a 250-mL Erlenmeyer flask with 500 μL of overnight ER2738 and one plaque from the plate were grown overnight at 37° C., shaking at 250 rpm. The next day the phage was precipitated the same as above and the pelleted phage were resuspended in ˜5 mL PBS buffer pH 7.4 to make a phage solution of ˜10¹² pfu/mL and stored at 4° C. for further use.

Primer 9:
(SEQ ID NO: 34)
5′-TACCAGCAGAGTGAGAATAGAAAG-3′
Primer 10:
(SEQ ID NO: 35)
5′-GTGCTTGTAGTTGGGGTATTGAG-3′

### 6.2 Modification of the N-Terminal Peptide on Phage Using Crosslinker 3b

The protocol for phage modification, isolation, and identification of the modified peptide from phage was the same for both phage variants and can be used for other M13 discreet phage variants that have a TEV protease cleavage site between the N-terminal peptide and the native pill protein residues. First, 1 mL of discreet phage (˜10¹² pfu/mL) was precipitated with 200 μL of 20% PEG/2.5 M NaCl for 1 hour on ice. The precipitated phage was pelleted by centrifugation (14,000 rpm, 20 minutes, 4° C.) and resuspended in 500 μL of buffer R (20 mM ammonium bicarbonate, pH 8.0). The phage was reduced with 1 mM TCEP for 1 hour at room temperature and then precipitated with ⅙ volume of 20% PEG/2.5 M NaCl on ice for 1 hour. The precipitated phage was pelleted by centrifugation (14,000 rpm, 20 minutes, 4° C.) and resuspended in 500 μL of PBS pH 7.4. The phage was then mixed with 10 μL of BC-OSu (3b) (1 mM stock solution in DMF), resulting in a total of 10¹² phage particles and 20 μM of crosslinker in 2% DMF/PBS buffer. The mixture was vortexed and incubated at room temperature for 20 minutes. The reaction was quenched with 1 mM cysteine for 30 minutes at room temperature and then the modified phage as precipitated with ⅙ volume of 20% PEG/2.5 M NaCl on ice for 1 hour. The precipitated phage was pelleted by centrifugation (14,000 rpm, 20 minutes, 4° C.) and resuspended in 100 μL of water.

To separate the modified peptide from the rest of the phage particles, the phage was incubated with 300 μL of TEV protease (10 μM stock in 50 mM Tris, 0.5 mM EDTA pH 8.0), resulting in 10¹² phage particles and 7.5 μM of TEV protease. The phage was cleaved overnight at 30° C. (˜16 hours). The next morning, an Amicon Ultra-0.5 Centrifugal Filter (Sigma Aldrich, #UFC505008) was used to separate the cleaved peptide from the rest of the phage particles. The solution was filtered by centrifugation (14,000 rpm, 25 minutes, RT) and the sample that flowed through the filter was lyophilized overnight. The lyophilized sample was resuspended in 30 μL of water and then analyzed by LC-MS (Method B).

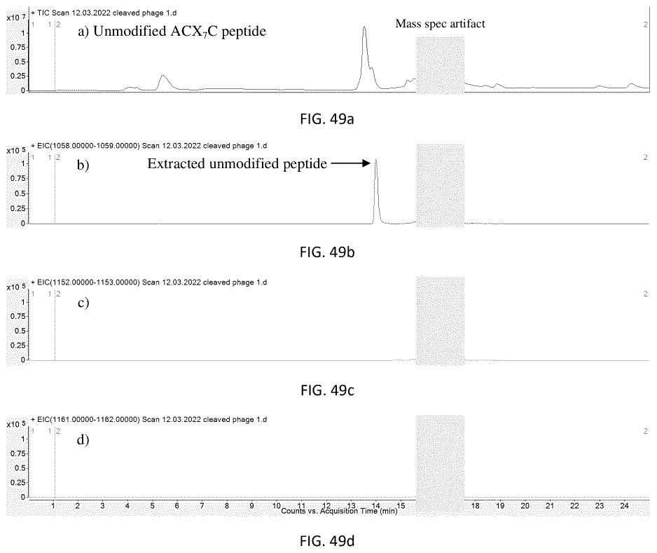

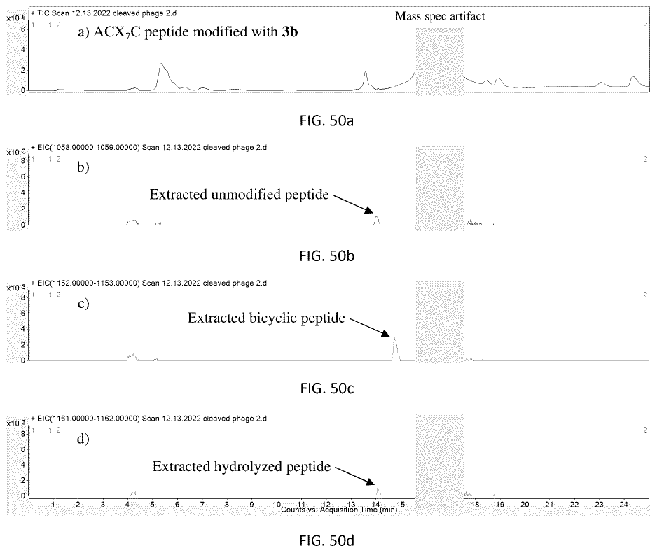

For each individual sample, the [M+2H]²⁺ mass range of the unmodified, bicyclic, and hydrolyzed peptides were extracted and the retention time and area under the peak was integrated and compared to each other to determine the percentage of modified peptide (Table 6). First as a control, the unmodified peptide from each of the phage variants were cleaved and analyzed by LC-MS to determine the retention time of the unmodified peptides. The same [M+2H]²⁺ mass ranges were extracted for the unmodified peptides to see if there was any artifact in the sample with the same masses as the modified peptides. With the unmodified ACX₇C peptide, it showed only unmodified peptide in the sample, with little background noise (FIGS. 49a-49d). When the ACX₇C phage was modified with freshly prepared crosslinker 3b, it was found that 21% of the peptide was left unmodified, 66% was bicyclic, and 13% was hydrolyzed (FIGS. 50a-50d). This modification on the ACX₇C phage was repeated four times and each trial gave very similar results.

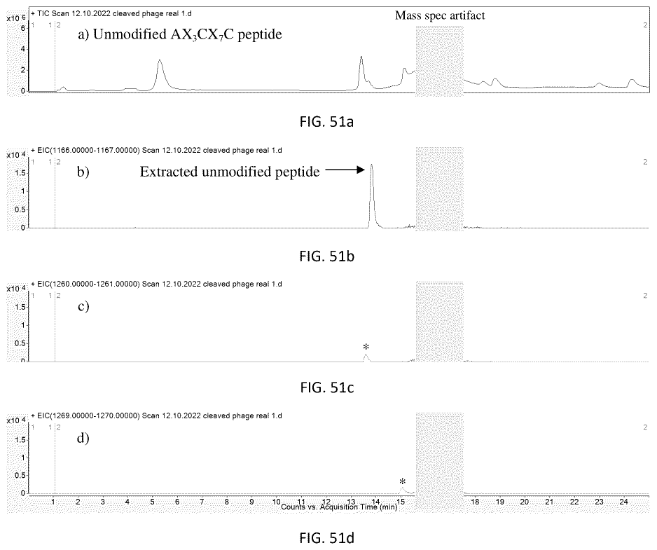

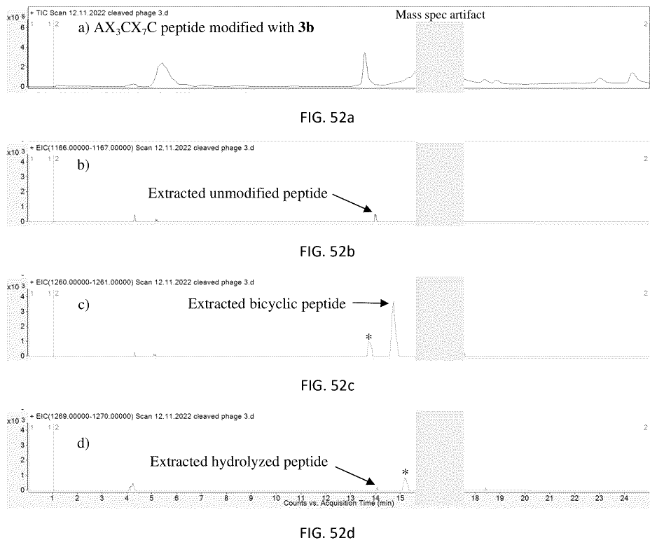

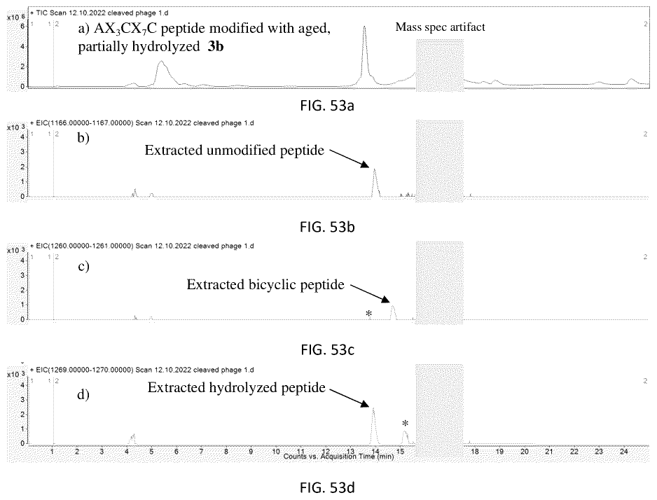

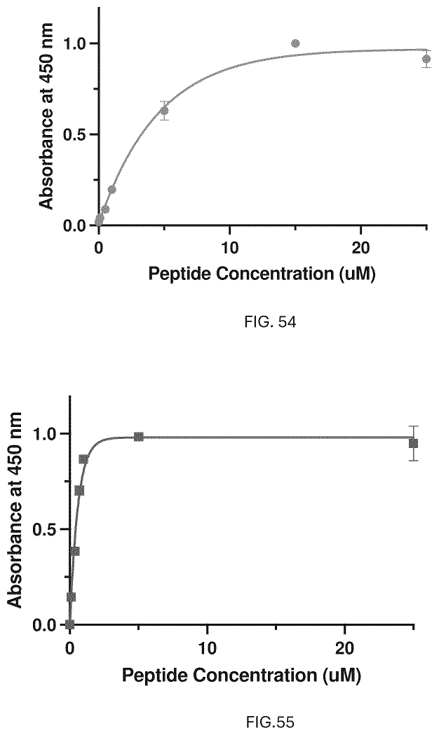

The [M+2H]²⁺ mass ranges were also extracted for the unmodified AX₃CX₇C peptide, however for this sample by extracting the bicyclic and hydrolyzed masses it did show small peaks that are mass spectrometry artifacts not associated with the cleaved peptide (FIGS. 51a-51d). When the AX₃CX₇C peptide was modified with freshly weighed and solubilized crosslinker 3b, it was found that 5% of the peptide remained unmodified, 94% was bicyclic, and 1% was hydrolyzed (FIGS. 52a-52d). This experiment was repeated once, and the results were very similar to the reported percentages. Although the extracted masses show other peaks, these were also seen in the unmodified sample and were identified as mass spectrometry artifacts (identified as * in the FIGs.). When the AX₃CX₇C phage was modified with aged, partially hydrolyzed crosslinker 3b, the percentage of bicyclic peptide decreased. It was found that 36% of the peptide remained unmodified, only 17% was bicyclic, and 47% was hydrolyzed (FIGS. 53a-53d). This experiment with aged 3b allowed for the identification of all three peptide species and indicated that the sample with fresh 3b does truly result in 94% of bicyclic peptide. These results show that the majority of phage were successfully modified with the freshly prepared crosslinker 3b and indicate that 3b can be used to construct bicyclic phage libraries for phage display screening against protein targets.

TABLE 6
Data from LC-MS analysis used to determine the
percentage of modified peptides on phage

| | | Extracted mass | Retention | Area under | |
|---|---|---|---|---|---|
| Sample | Peptide | [M + 2H]²⁺ M/Z | time (mins) | the peak | Percentage |
| Unmodified | Unmodified | 1058-1059 | 13.986 | 991324.2 | 100% |
| ACX₇C | Bicyclic | 1152-1153 | N/A | N/A | N/A |
| (FIGs. | Hydrolyzed | 1161-1162 | N/A | N/A | N/A |
| 49a-49d) | | | | | |
| Modified | Unmodified | 1058-1059 | 13.992 | 9817.76 | 21% |
| ACX₇C | Bicyclic | 1152-1153 | 14.754 | 30817.35 | 66% |
| (FIGs. | Hydrolyzed | 1161-1162 | 14.058 | 6317.81 | 13% |
| 50a-50d) | | | | | |
| Unmodified | Unmodified | 1166-1167 | 13.829 | 176457.6 | 100% |
| AX₃CX₇C | Bicyclic | 1260-1261 | 13.580 | Not peptide | N/A |
| (FIGs. | Hydrolyzed | 1269-1270 | 15.072 | Not peptide | N/A |
| 51a-51d) | | | | | |
| Modified | Unmodified | 1166-1167 | 14.005 | 2201.53 | 5% |
| AX₃CX₇C | Bicyclic | 1260-1261 | 13.740 | Not peptide | N/A |
| (FIGs. | | | 14.718 | 38284.48 | 94% |
| 52a-52d) | Hydrolyzed | 1269-1270 | 14.071 | 428.02 | 1% |
| | | | 15.165 | Not peptide | N/A |
| Modified | Unmodified | 1166-1167 | 13.955 | 18971.52 | 36% |
| AX₃CX₇C | Bicyclic | 1260-1261 | 14.684 | 9065.23 | 17% |
| (FIGs. | Hydrolyzed | 1269-1270 | 13.905 | 24957.32 | 47% |
| 53a-53d) | | | 15.148 | Not peptide | N/A |

### Example 7

### Design and Evaluation of Bicyclization Protocols

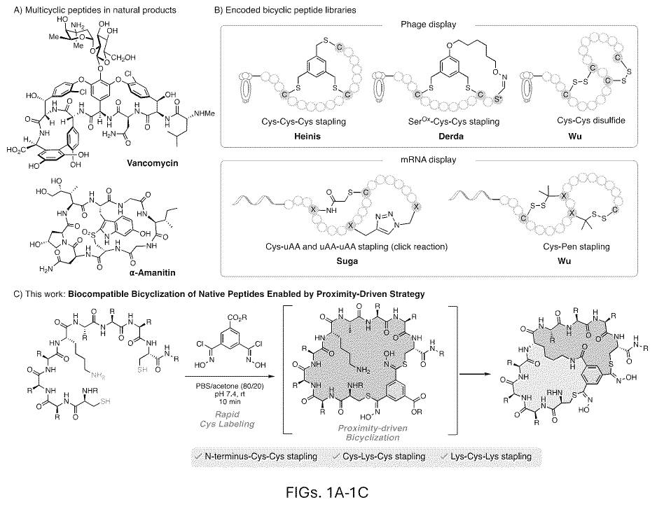

It was set out to develop a protocol to enable native peptide bicyclization under physiologic conditions, with the ultimate goal of applying this protocol to build multicyclic peptide libraries. The method would allow construction of a bicyclic structure by crosslinking diverse residues instead of just cysteines and suppress side reactions and peptide oligomerization. A multi-functionalized crosslinker with tiered reaction rates towards different protein residues would allow cascade reactions with a proper peptide sequence. For example, the linear to bicycle transformation could initiate with a rapid Cys-Cys stapling to form a monocyclic intermediate, which sets up a proximity-driven amine coupling to give bicyclic peptides (FIG. 1C). A fast and highly specific cysteine conjugation reaction^{[14-17]} is the key to achieving this cysteine-directed strategy. It was recently reported chlorooxime-based reagents that conjugate with cysteine thiol with fast kinetics (k₂=306±4 M⁻¹s⁻¹),^{[15]} which appear to be an ideal candidate for developing cysteine-directed crosslinkers. For the second step of cyclization, an amide bond formation was chosen considering the prevalence and importance of amide bond in peptide and protein structures, and the availability of amine functionalities in lysine^{[18]} and N-terminus^{[19]}.

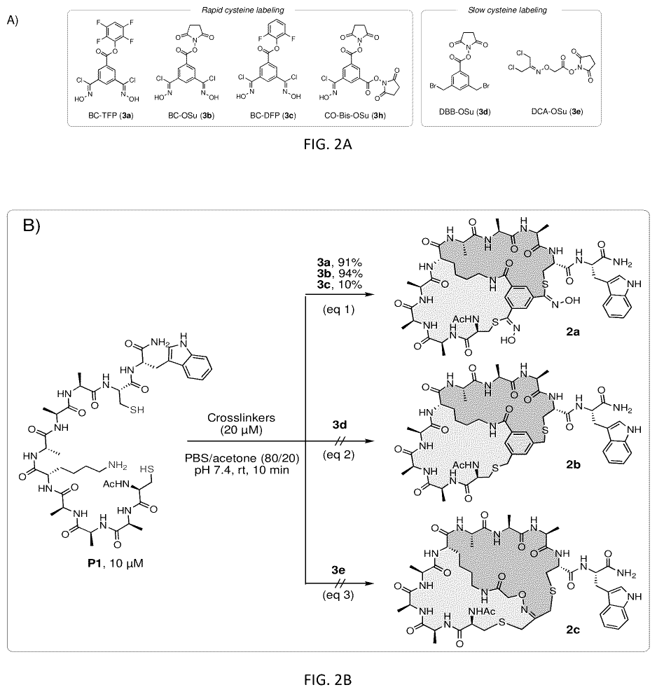

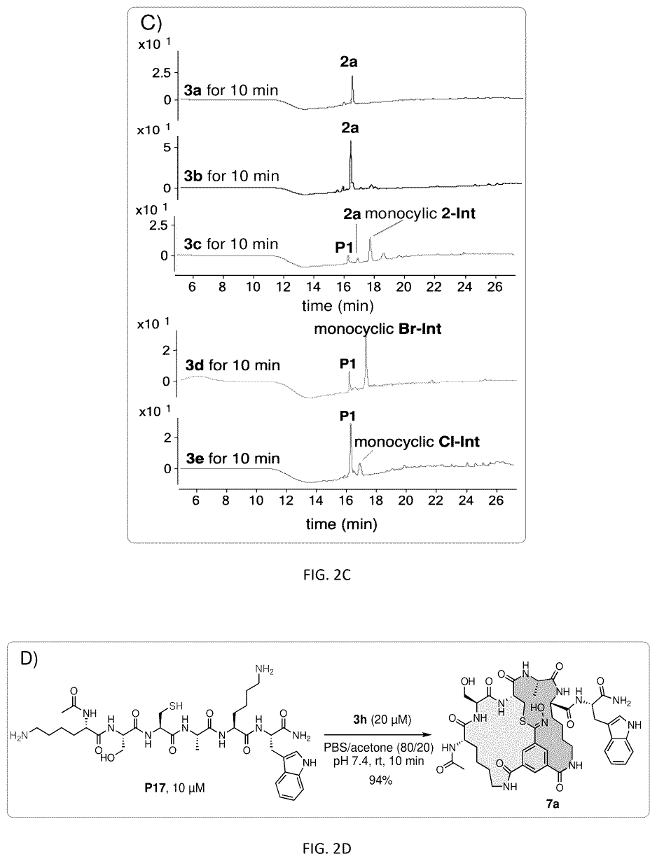

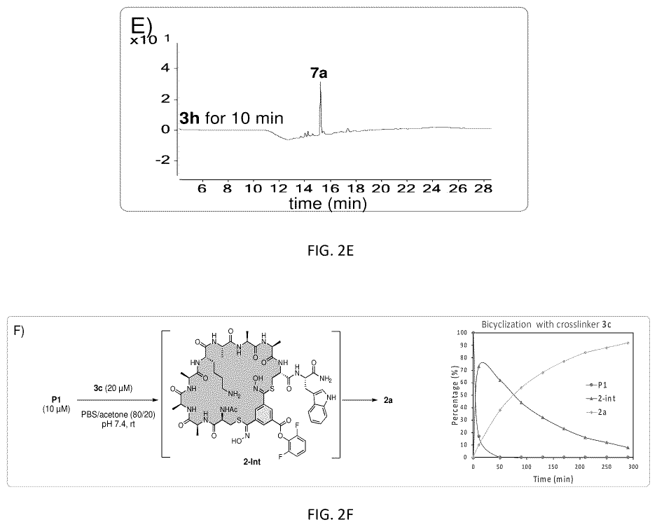

A series of bifunctional reagents have been recently reported for accessing monocyclic peptides via Cys-Lys crosslinking.^{[7d, 20]} With these considerations in mind, a panel of tri-functional crosslinkers 3a, 3b, and 3c were designed that install a rapid cysteine-reactive bischlorooxime (BC) motif and an activated ester for amine coupling (FIG. 2A). For comparison, 3d and 3e that bear a dibromomethyl benzene (DBB), and 1,3-dichloroacetone (DCA) motif respectively were also synthesized. DBB^{[21]} and DCA^{[22]} are commonly used for cysteine conjugation, however, display slow reaction kinetics in comparison to the chlorooxime reagent.^{[15]} Additionally, crosslinker 3 h with a single chlorooxime motif and two activated esters was also prepared. Both the thiol and amine crosslinking reactions involved could be carried out in native peptides under physiological conditions.

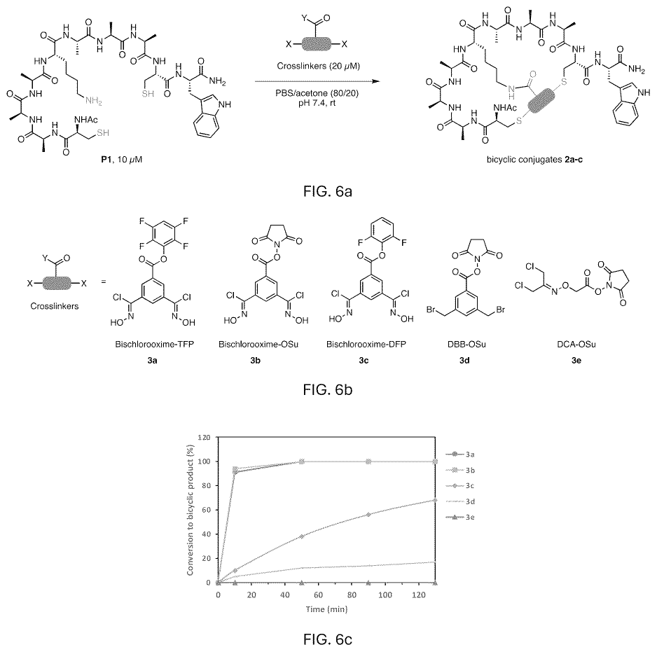

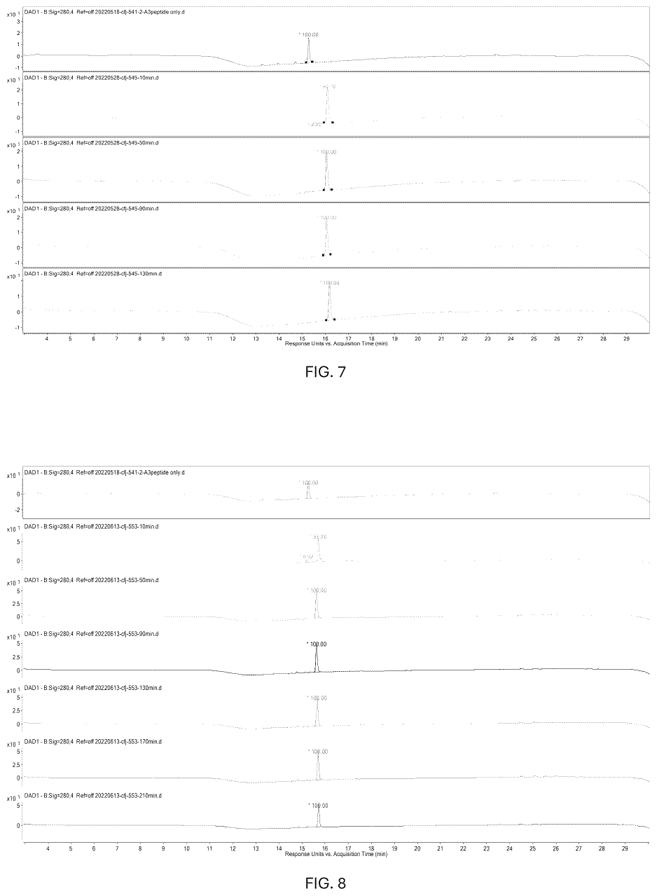

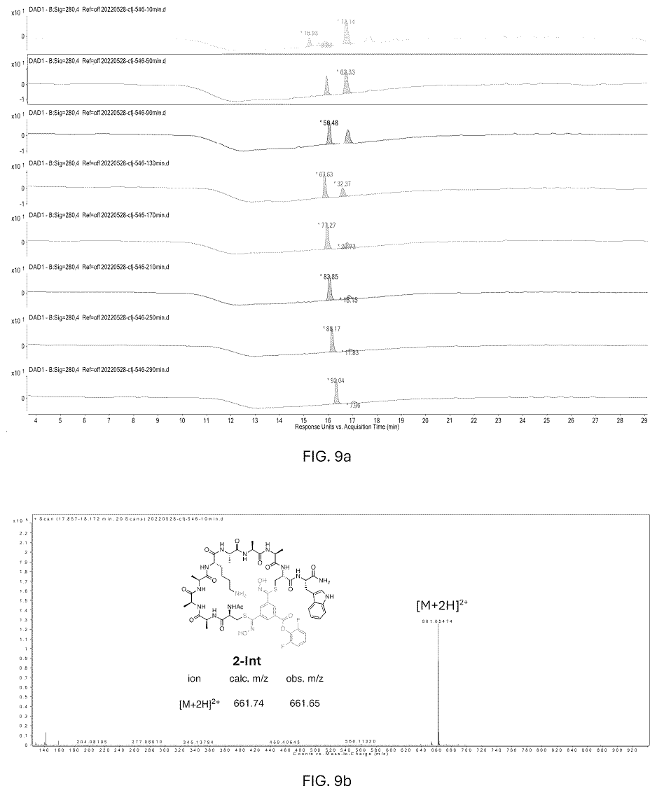

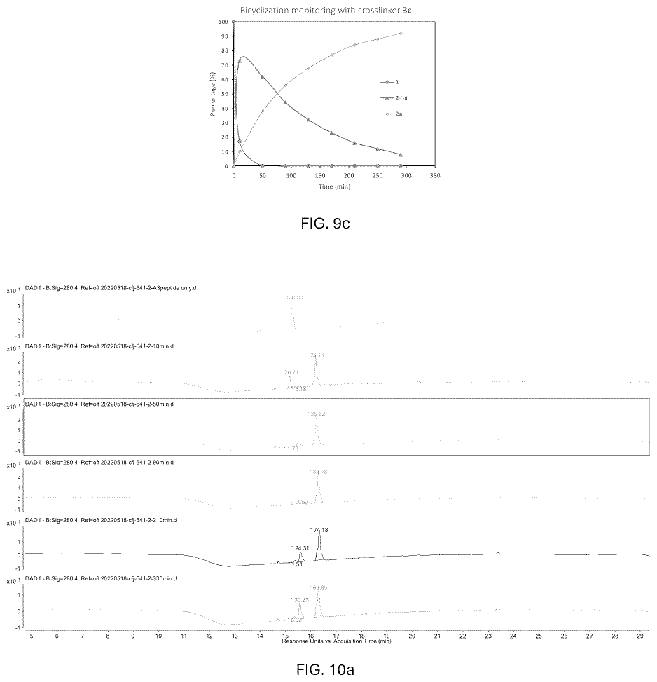

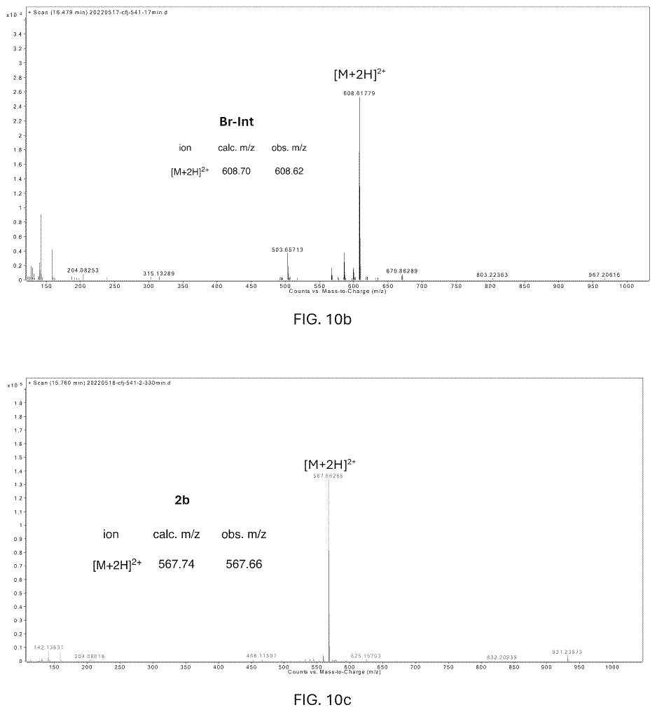

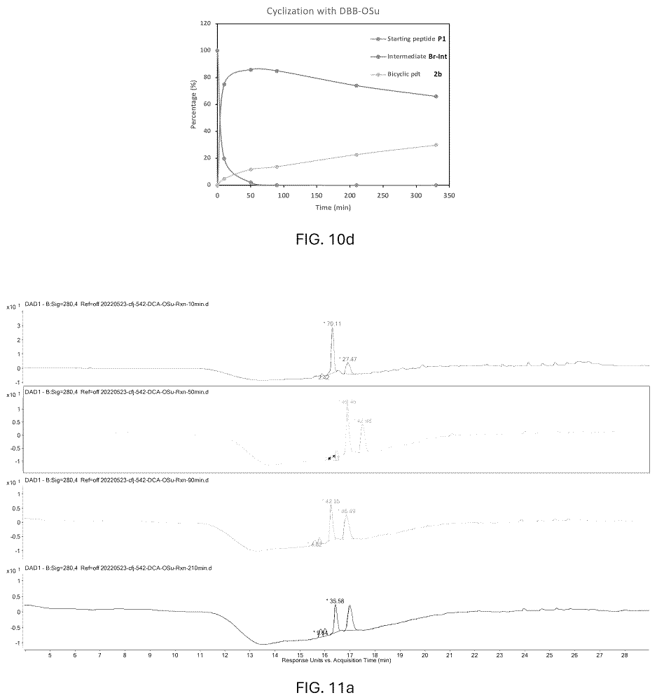

Crosslinkers 3a-e were comparatively evaluated for their capability of bicyclizing native peptides. These crosslinkers were incubated with a model peptide (Ac)CA₃KA₃CW-CONH₂ (SEQ ID NO: 36) (P1) under mild conditions (PBS with 20% acetone for solubility of the crosslinker, pH 7.4, room temperature), and the reactions were monitored using LC-MS (FIGS. 2A-2F). All of the bischlorooxime-containing crosslinkers (3a, 3b, and 3c) generated the bicyclic product 2a smoothly through Cys-Lys-Cys stapling. Among them, 3a with tetrafluoro phenol ester (TFP) and 3b with N-succinimidyl ester (NHS or OSu) worked with equal efficiency, generating bicyclic peptide 2a with near-complete conversion within 10 minutes (FIGS. 2B & 2C; FIGS. 6-8). Crosslinker 3c with a difluorophenol ester (DFP) took a longer time (5 hours) to reach complete bicyclization, which is not surprising considering its weaker leaving ability for amino nucleophilic attack compared to TFP and NHS (FIG. 2F, FIGS. 9a-9c). The observed correlation between the ester reactivity and the cyclization rate affords bicyclization protocols with tunable reaction kinetics. Activated esters 3d and 3e, which contain slow cysteine labeling handles, failed to generate a significant amount of bicyclic product (FIG. 2B, eq 2, 3). Instead, they elicited either low conversion or gave monocyclic peptide bearing intact alkyl halide as the major product (FIG. 2C; FIGS. 10a-10d; FIGS. 11a-11c). In addition to the Cys-Lys-Cys stapling, Lys-Cys-Lys stapling of peptide P17 was also achieved to give bicyclic 7a with 94% conversion when using crosslinker 3h (FIGS. 2D & 2E). These results proved that peptide bicyclization can be achieved by kinetic control of sequential residue modifications.

### Example 8

### Mechanistic Studies

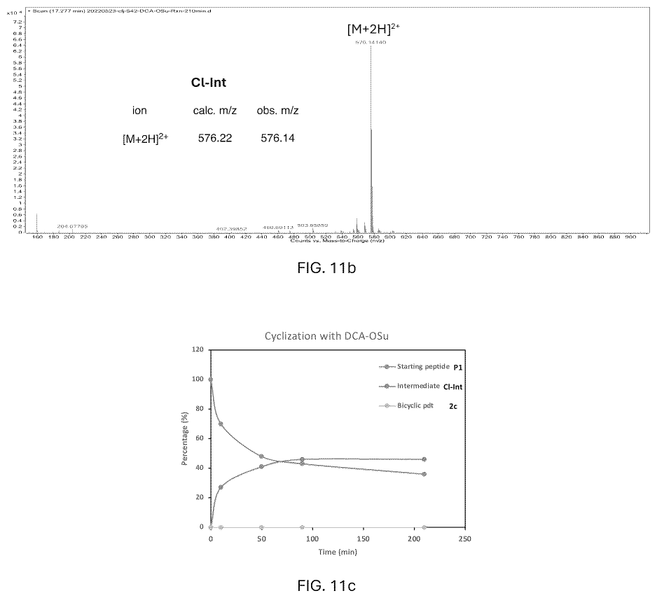

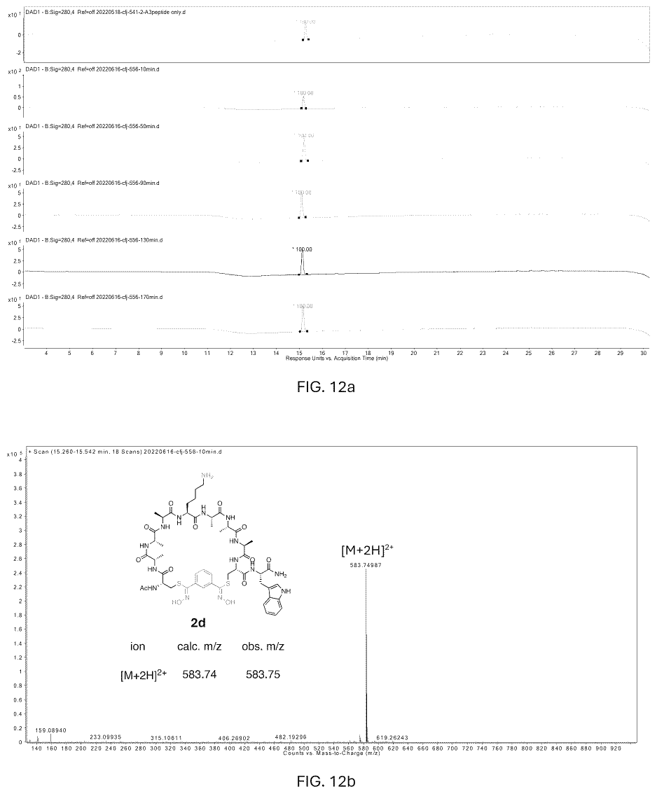

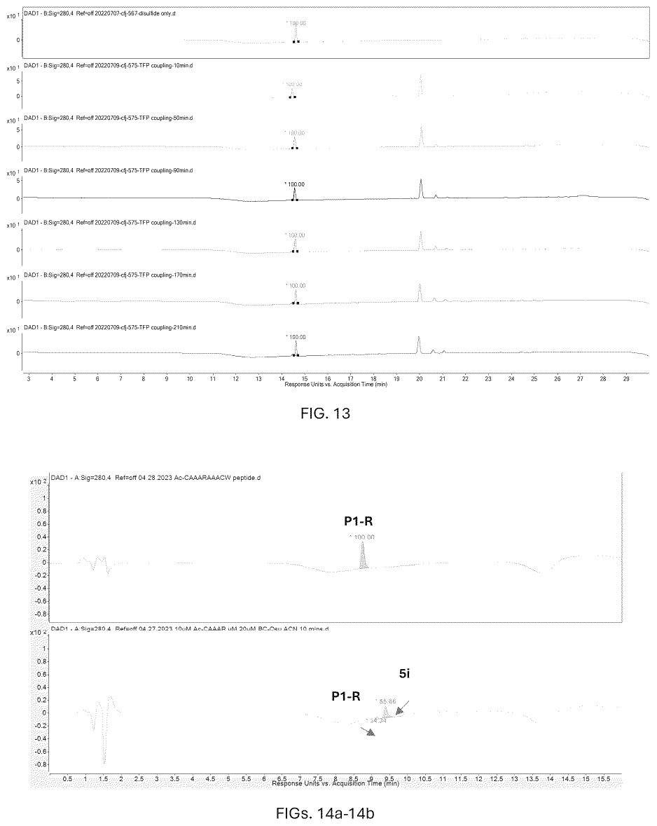

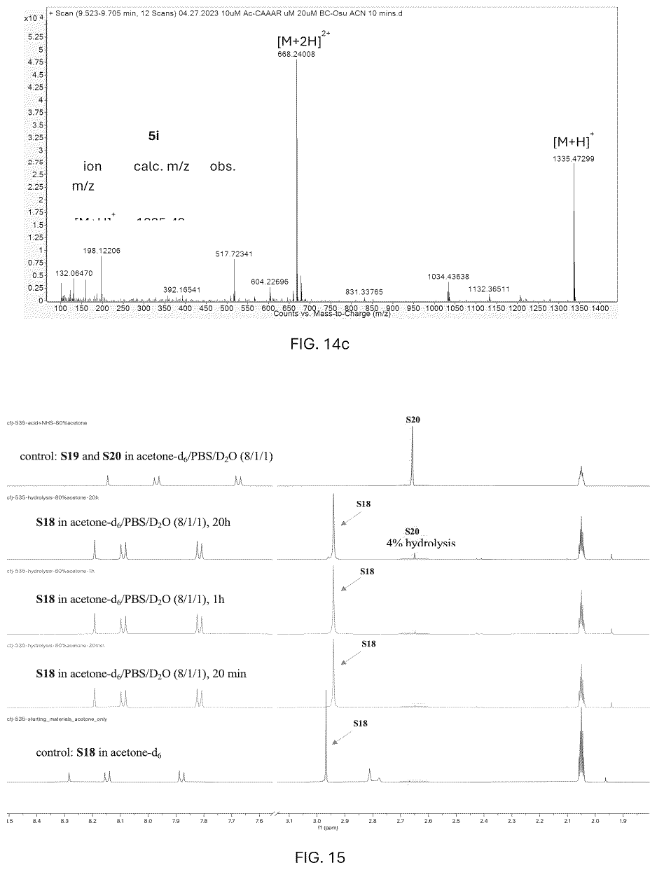

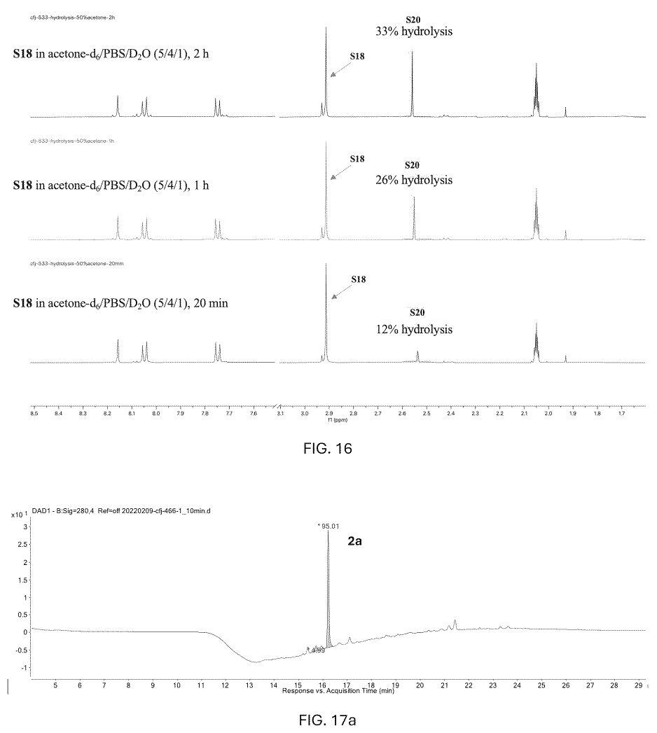

To gain deeper insight into the reaction mechanism, control experiments and kinetics studies were carried out. In a control experiment, cyclization of peptide P1 using bischlorooxime 3f gave Cys-Cys stapled product with full conversion within 10 minutes (FIGS. 12a-12b). Under identical reaction conditions, TFP ester 3g failed to couple with lysine residue of model peptide P1-disulfide without the promotion of cysteine labeling (FIG. 13). On the contrary, bischlorooxime-containing TFP ester 3a was able to accomplish lysine coupling efficiently in 10 minutes time scale (FIG. 2B, eq 1). These results indicate the bicyclic product was generated through proximity-driven lysine condensation that was enabled by Cys labeling, which increases the local concentration of ester and amine by installing the crosslinker to peptide substrate to favor intramolecular amide bond formation. This pathway was further supported by the observation of monocyclic intermediate 2-int (FIG. 2F, FIGS. 9a-9c), which was generated rapidly from the slow amine-reacting crosslinker 3c, and then gradually converted into the bicyclic product 2a. These results demonstrated the crucial role of the rapid chlorooxime-cysteine conjugation in proximity-driven peptide bicyclization. The chlorooxime-thiol conjugation involves a water-assisted nitrile oxide formation via hydrochloric acid elimination (Scheme S3) as descried in the earlier report.^{[15,23]}

### Example 9

### Substrate Scope

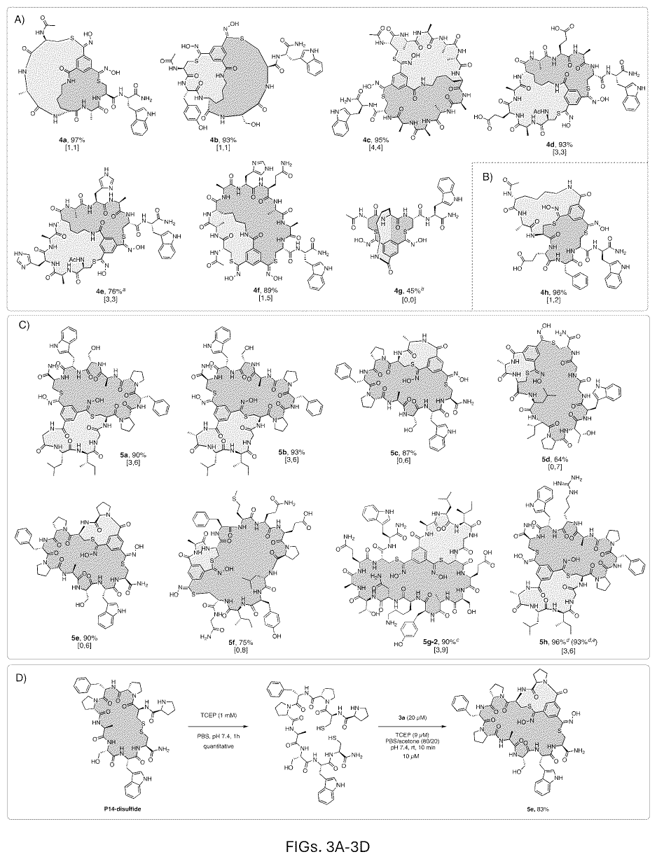

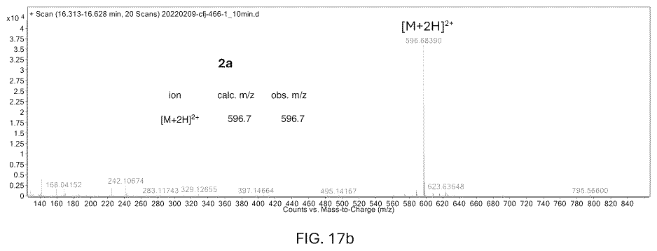

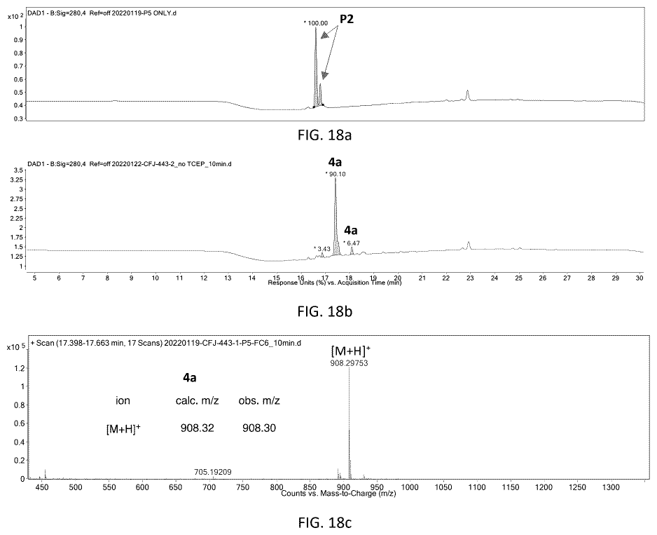

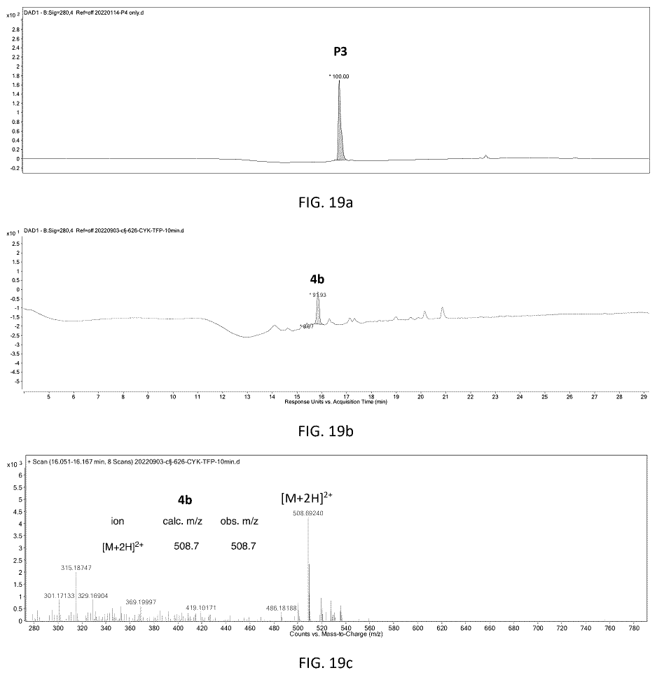

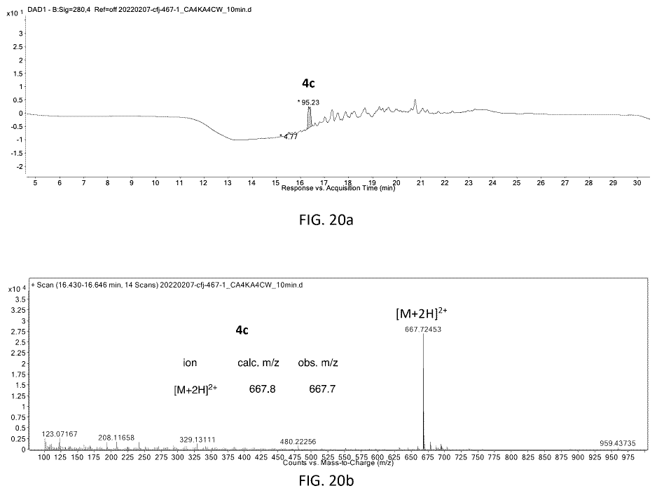

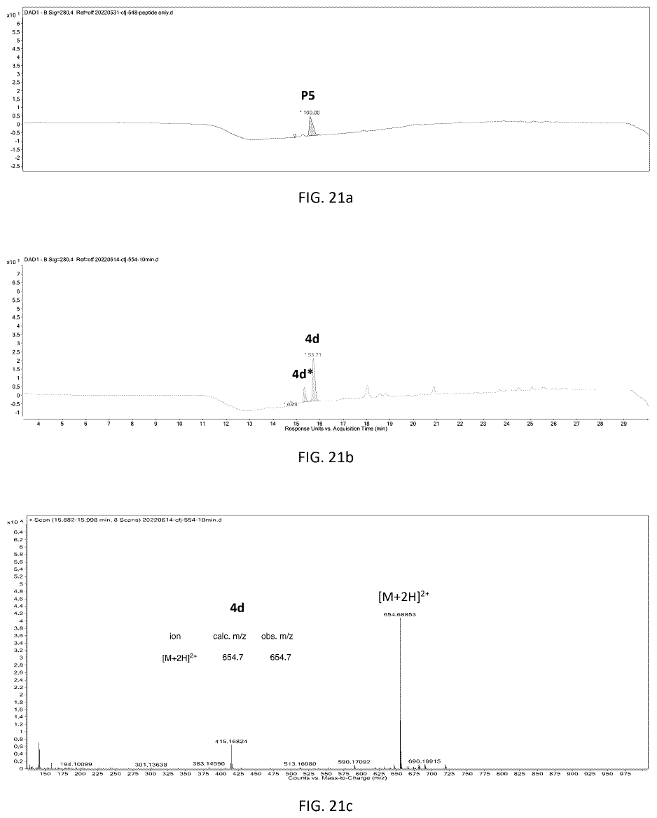

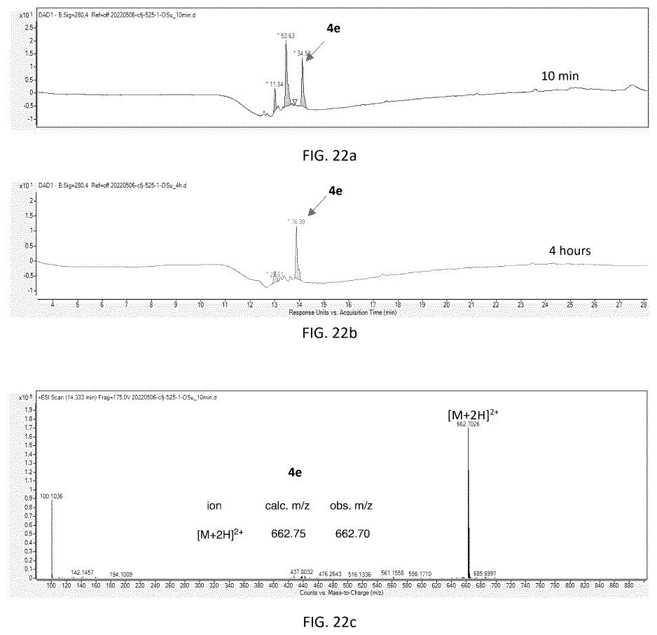

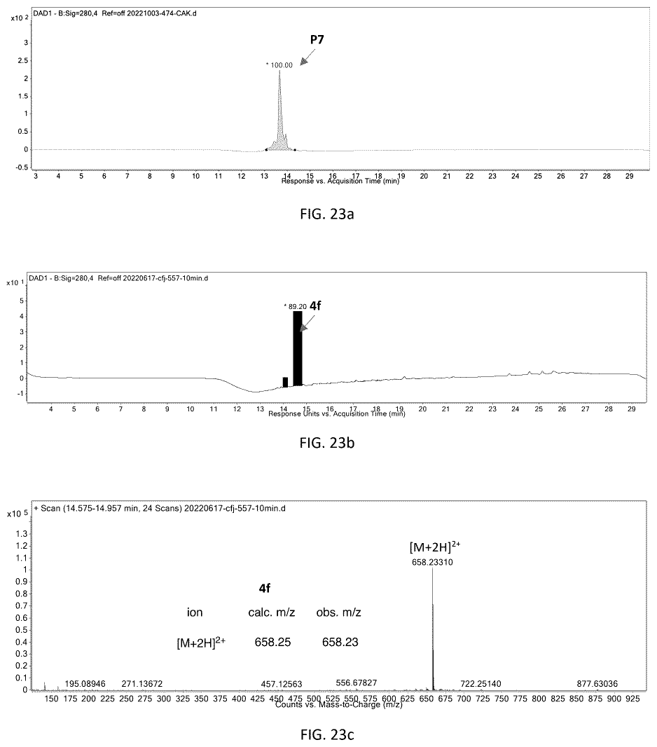

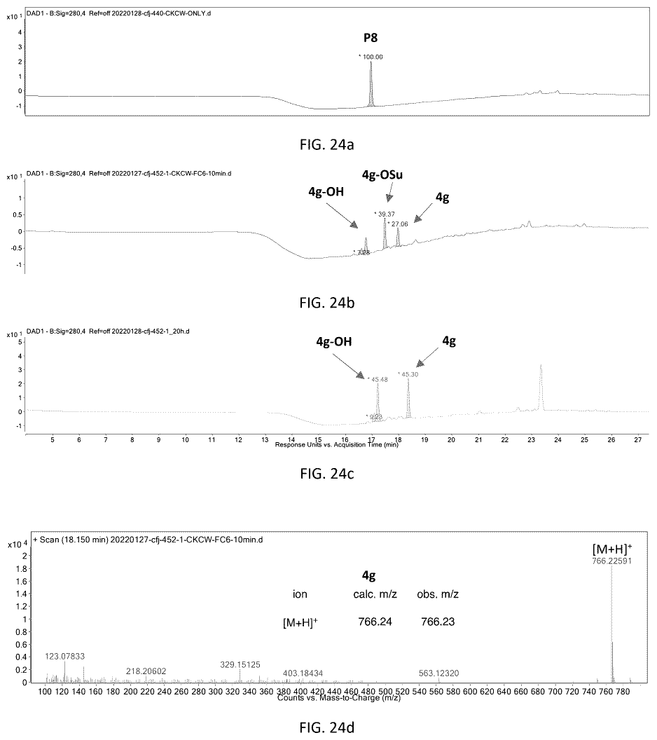

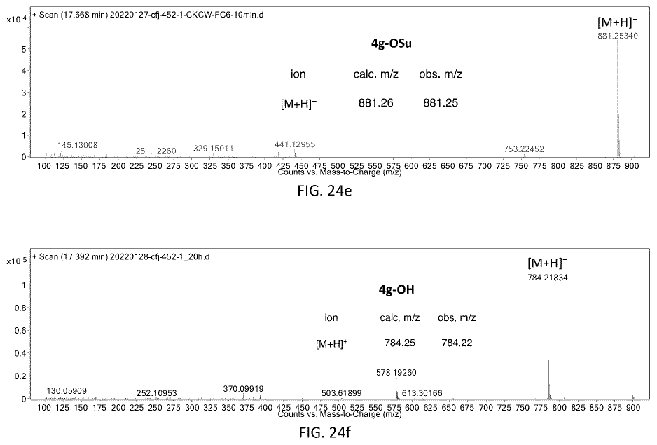

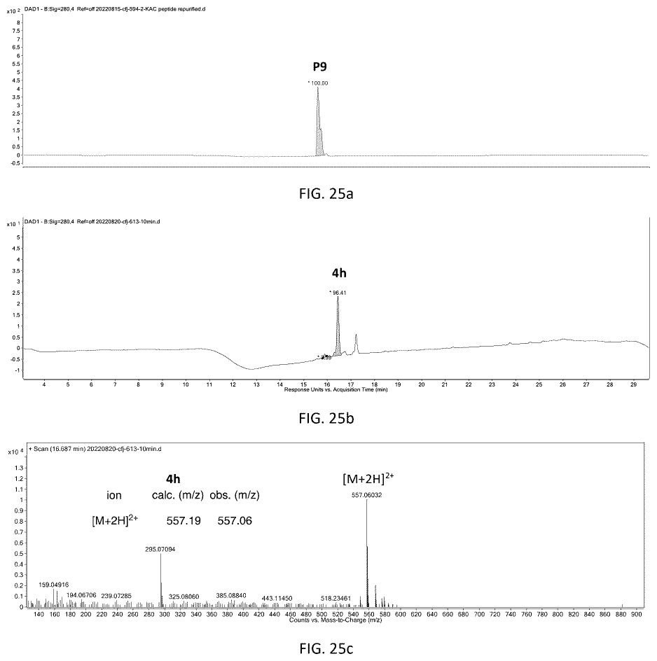

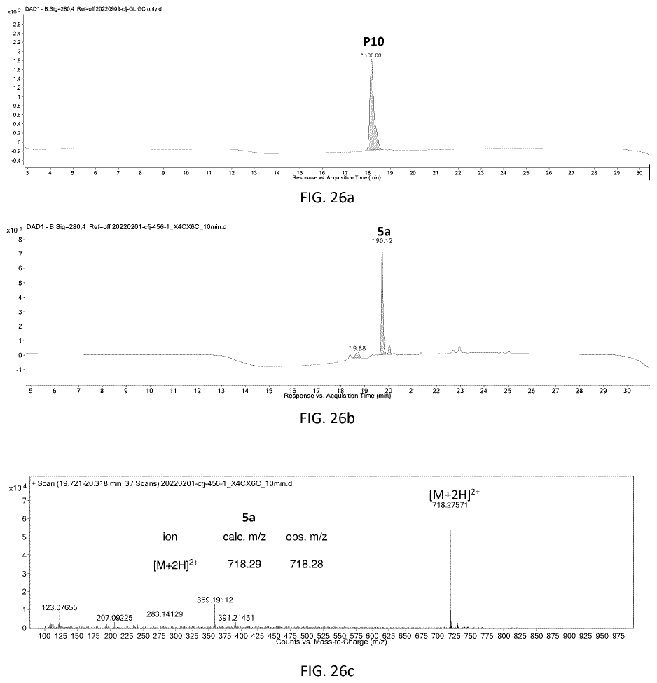

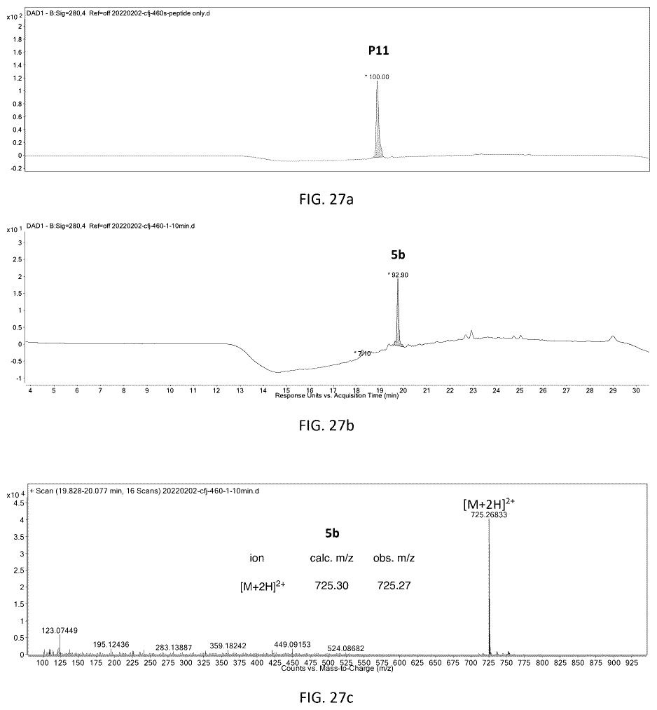

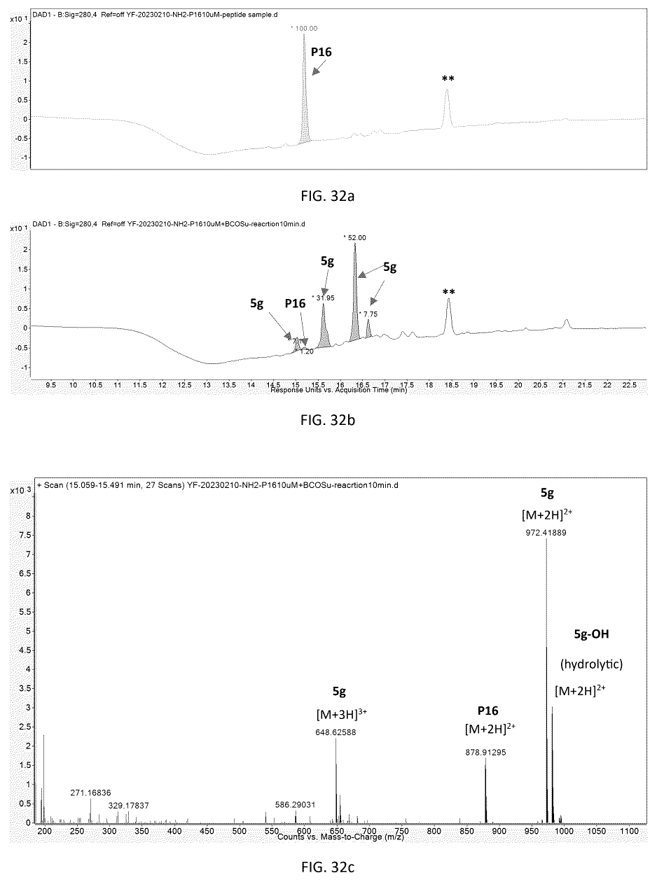

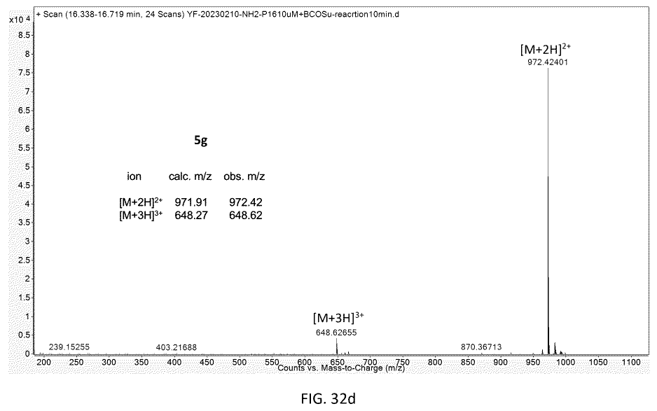

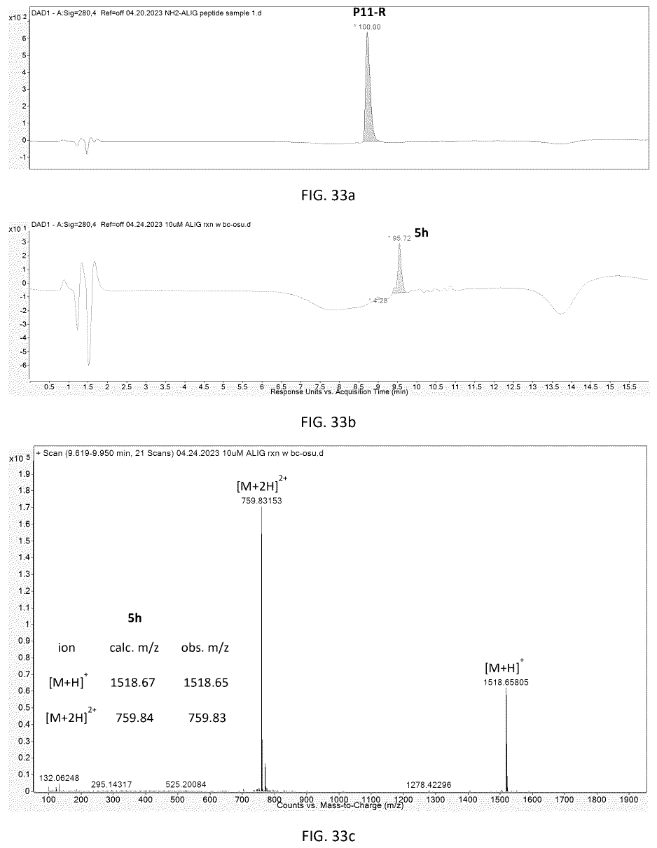

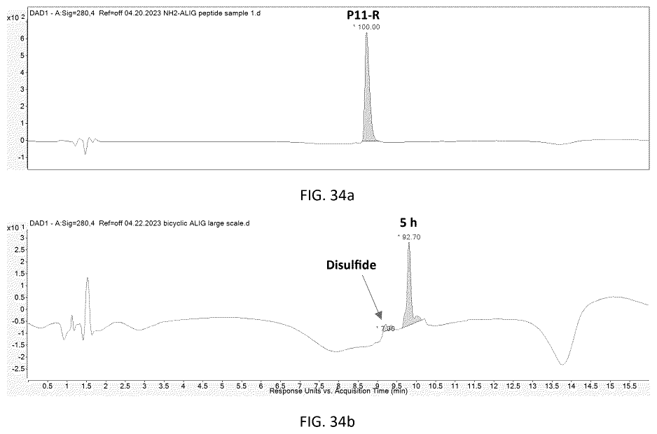

Encouraged by the initial results, this protocol was further expanded for the bicyclization of various linear native peptides. A wide range of butterfly-shaped bicyclic peptides were constructed with high efficiency using crosslinkers 3a or 3b (FIGS. 3A-3D; FIGS. 17a-17b; 18a-18c; 19a-19c; 20a-20b; 21a-21c; 22a-22c; 23a-23c; 24a-24f; 25a-25c; 26a-26c; 27a-27c; 28a-28c; 29a-29d; 30a-30b; 31a-31c; 32a-32d; 33a-33c; 34a-34b; 35a-35c; 36a-36f; 37a-37c; and 38). The bicyclization shows high specificity for cysteine/amine-containing residues (lysine or N-terminus) over other endogenous amino acid residues, such as tryptophan (2a), serine (4b), tyrosine (4b), glutamine (4f) and glutamic acid (4d) (FIG. 3A). Histidine residue was normally considered to be more reactive than lysine residue towards activated ester,^{[24]} yet it appeared to be also tolerated in the bicyclization protocol (4e, 4f). It is possible that the acyl imidazole intermediate formed by activated ester-histidine coupling could further react with a nearby lysine 8-amine to generate the stable lysine bicyclized product as seen by the slowed formation of 4e (FIGS. 22a-22c).^{[25]} Topologically, side chains bicyclic peptide structures were constructed through endo-lysine Cys-Lys-Cys stapling (FIG. 3A) and exo-lysine Lys-Cys-Cys stapling (FIG. 3B), respectively. Backbone-involved bicyclic products (5a-5g) were prepared through N-terminus-Cys-Cys stapling from native peptides bearing free N-terminus, such as Gly (5a), Ala (5b-d), and even sterically hindered Pro (5e) (FIG. 3C). The numbers [m, n] of spacer amino acids between modified residues could vary from 0 to 9, providing a wide range of structurally diversified macrocyclic peptides. Surprisingly, a highly constrained bicyclic tripeptide 4g with zero spacer amino acid ([m,n]=[0,0]) was obtained which involved only three amino acids in the core structure of the bicycle. This small size bicyclic tripeptide might have unique properties considering its rigid geometry and hydrophobicity.^{[26]} Next, the competitive reactivity of a lysine 8-amine and N-terminus α-amine were investigated. The result showed that both amine groups work efficiently, affording a mixture of two bicyclic products (5g-1/2, FIGS. 32a-32d). Although having multiple amines can complicate the reaction, arginine is well tolerated by the bicyclization protocol as demonstrated by two arginine-containing peptides P1-R and P11-R (FIGS. 14b-14c; FIGS. 34a-34b).

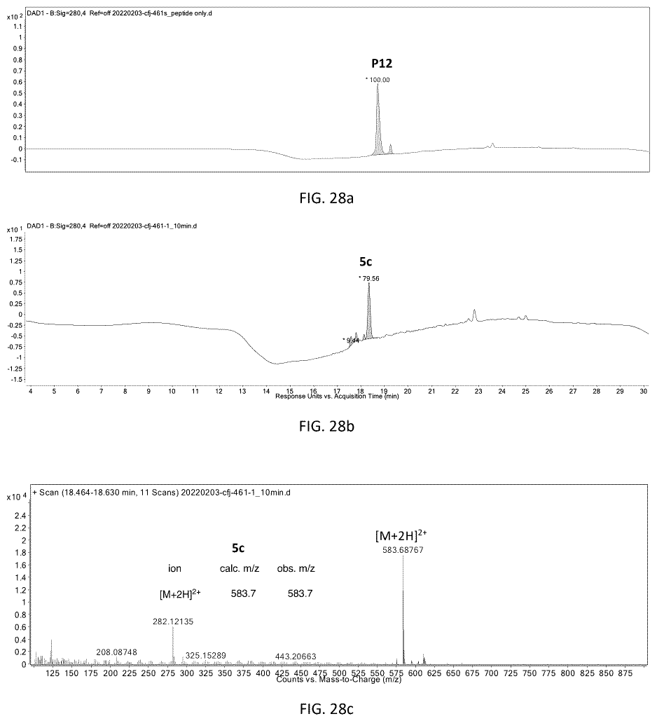

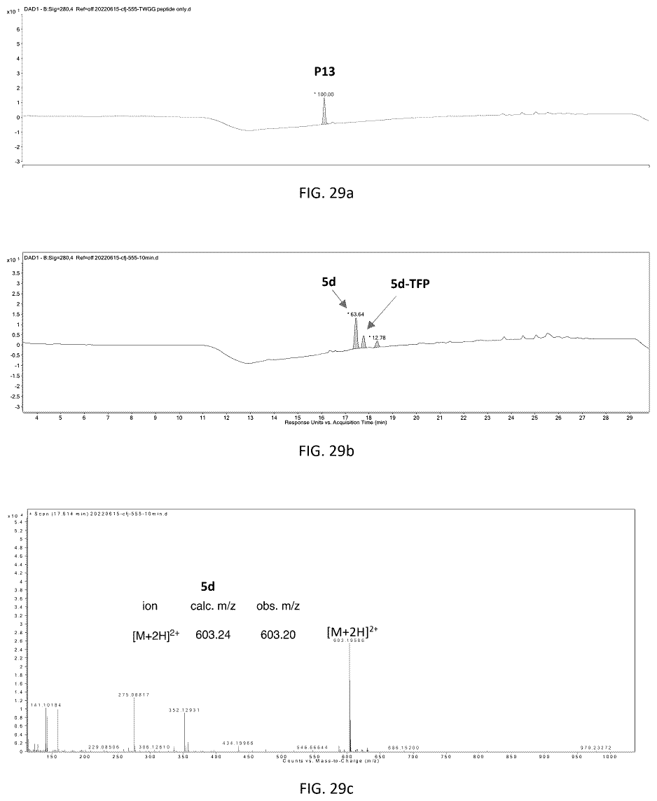

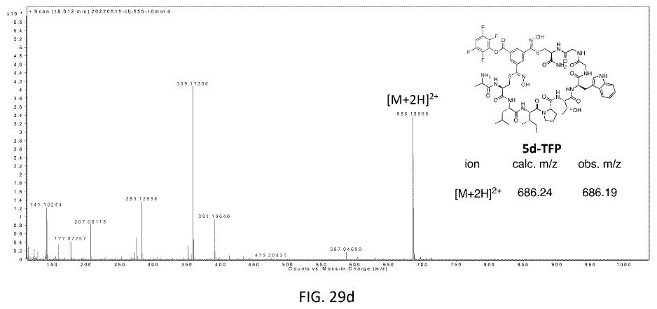

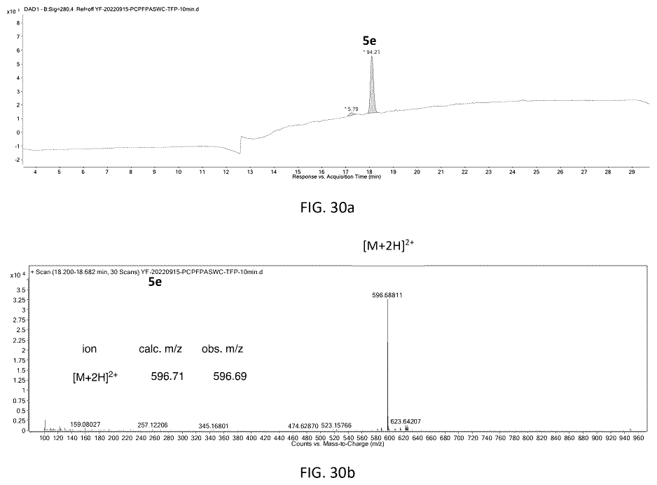

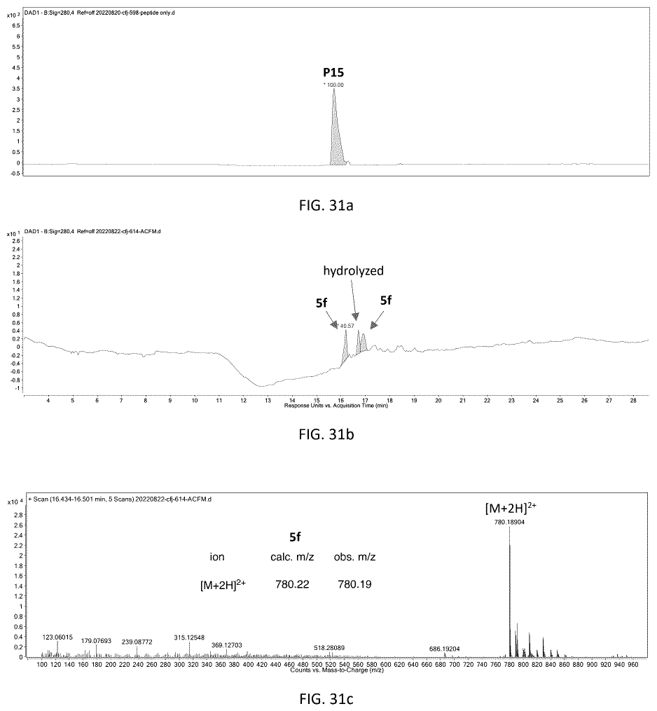

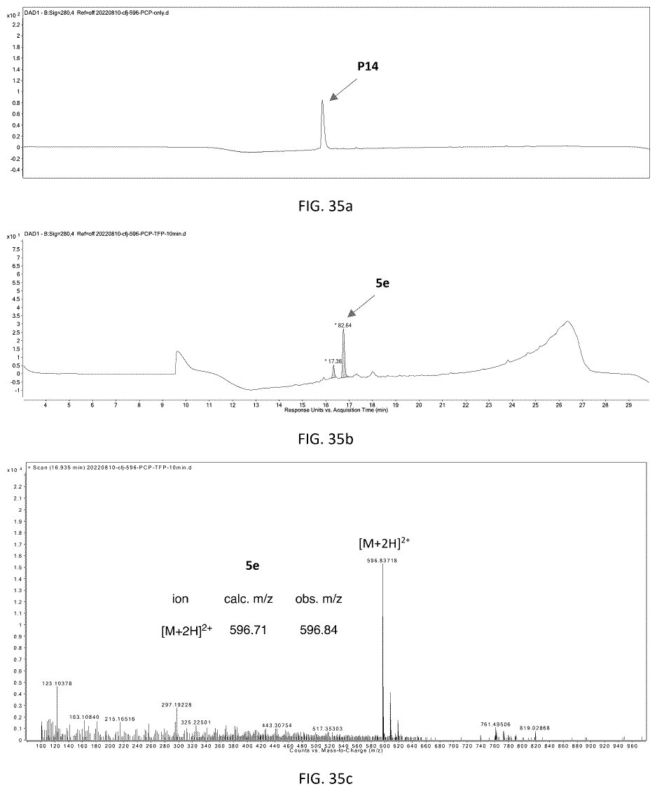

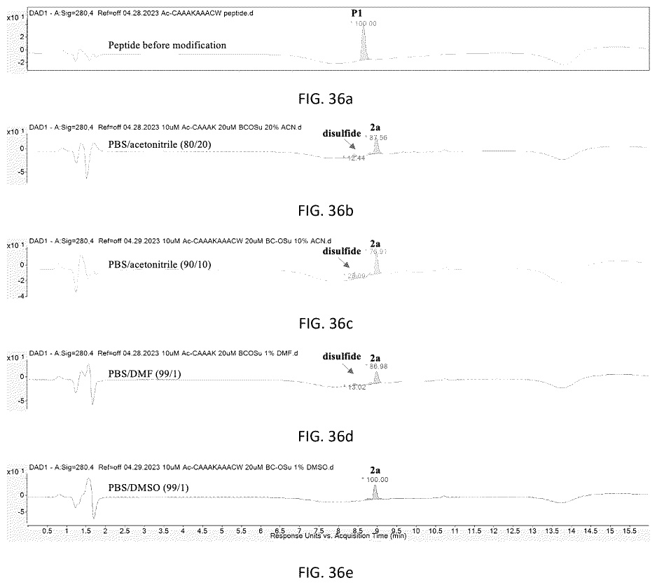

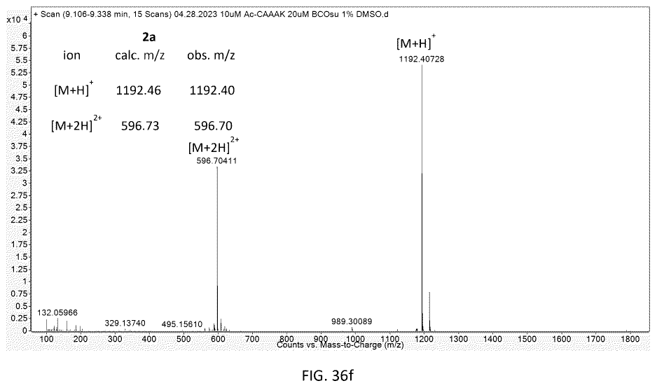

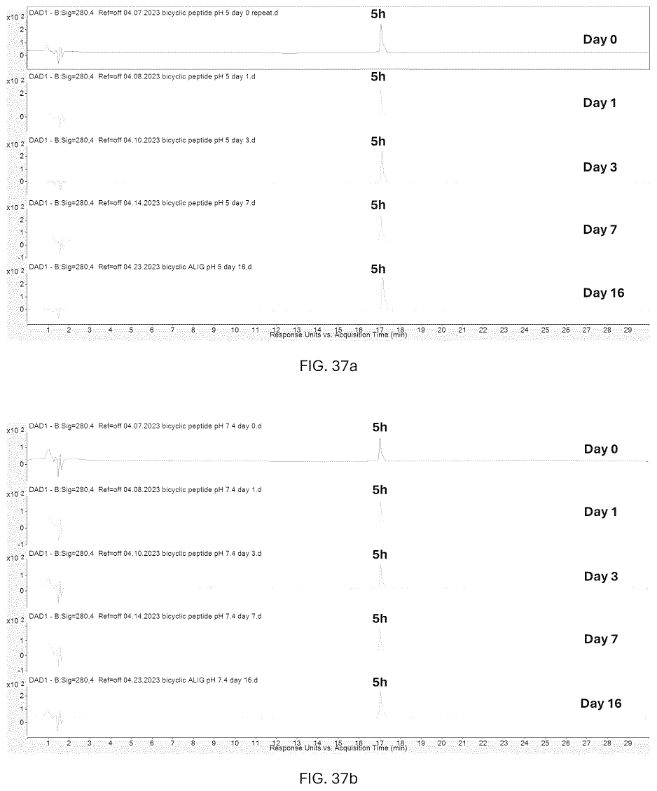

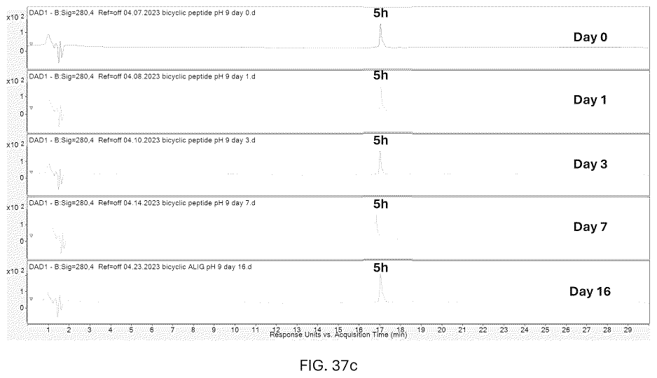

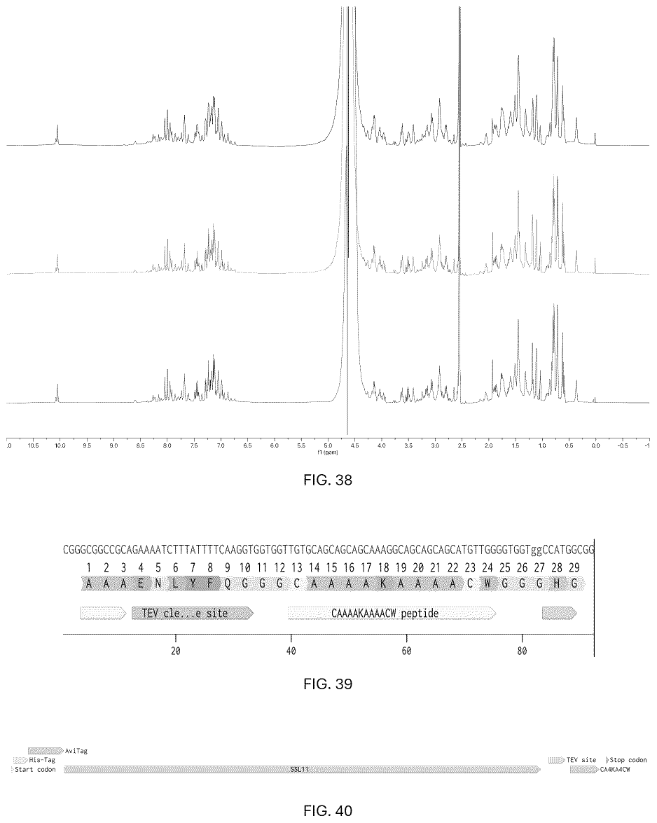

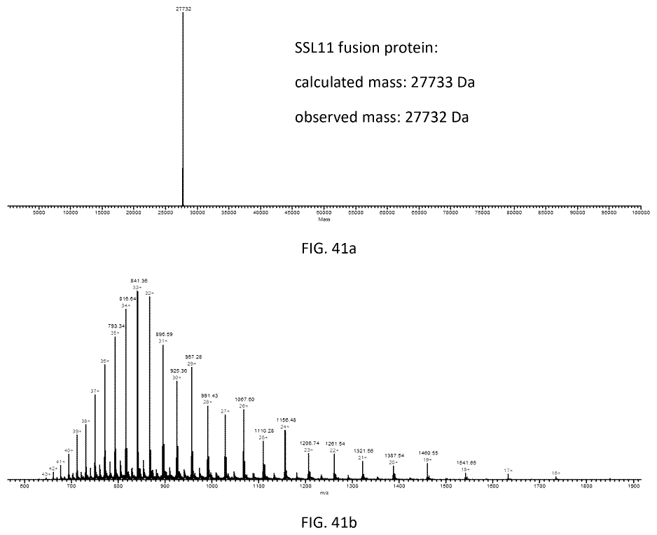

Although the above-mentioned examples were all carried out at low μM concentrations, the bicyclization protocol does allow reaction scaling up, producing comparable yield at mM concentrations of peptides (5h in FIG. 3C; FIGS. 34a-34b). Furthermore, efficient bicyclization can be achieved with the crosslinker stock solution prepared in various solvents, which afford mild and biocompatible bicyclization conditions including 1% DMSO (FIGS. 36a-36f). Importantly, tris (2-carboxyethyl) phosphine (TCEP) is well tolerated by the bicyclization protocol: disulfide cyclized peptides could be readily reduced by TCEP reduction and then subjected to bicyclization without purification to give the desired bicyclic products in high yields (FIG. 3D; FIGS. 35a-35c). Finally, and expectedly, peptides bicyclized using the protocol demonstrate robust chemical stability in aqueous media of varied pH (FIGS. 37a-37c; FIG. 28).

### Example 10

### Site-Specific Bicyclization of a Fusion Protein

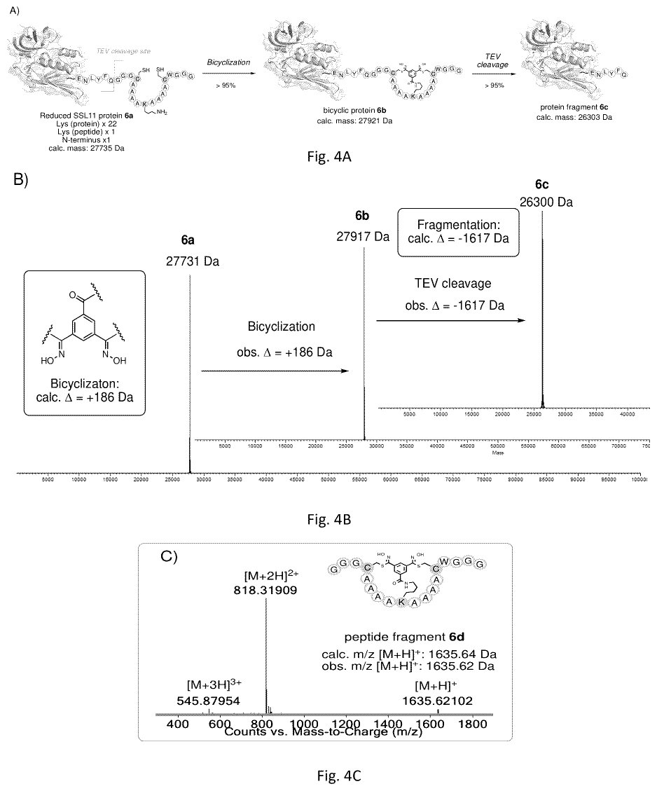

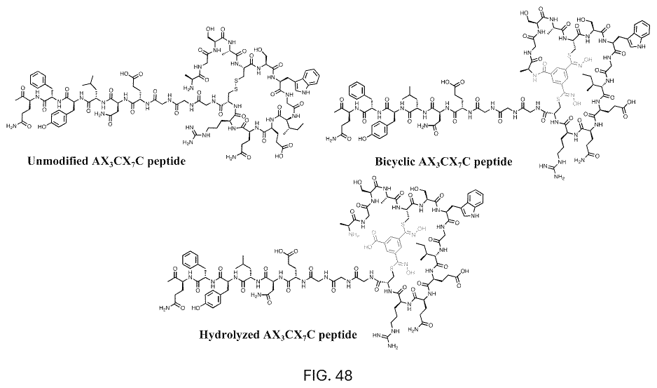

To examine the site-specificity of the bicyclization protocol, the bicyclization on protein-fused peptides was conducted (FIGS. 4A-4C; 41a-41b; 42a-42b; 43a-43b; 44a-44c; and 45a-45b). Specifically, a Staphylococcal Superantigen-like Protein 11 (SSL11)^{[27]} protein which fuses a model peptide (GGCA₄KA₄CWGGG (SEQ ID NO: 37)) to its C-terminus with a Tobacco Etch Virus (TEV) cleavage site (ENLYFQ/G) (SEQ ID NO: 38) in between^{[28]} were designed and expressed. The fusion protein also carries an N-terminal His-tag to facilitate purification. In addition to the lysine residue at the model peptide tail, this fusion SSL11 protein has 22 surface-exposed lysine residues and one free N-terminus (histidine), which could be the competitive nucleophiles for labeling. After reducing the disulfide with TCEP, the fusion SSL11 protein 6a was incubated with 2 equivalents of crosslinker 3b under the mild condition (8% acetone in PBS, pH 7.4, room temperature) (FIG. 4A). The reaction progress was monitored by LC-MS analysis. After incubation for 15 min, protein 6a was completely consumed, and a modified protein 6b with the molecular weight increased by 186 Da was obtained (FIG. 4B). This mass difference aligns with the calculated mass change for the peptide bicyclization (+186 Da). Over modification of the protein by the crosslinker was not observed. Similarly, the crosslinker 3a also elicited rapid and clean bicyclization of the reduced protein (FIG. 48). Treating the bicyclized protein with TEV protease cleaved the bicyclic peptide off the modified protein 6b. Deconvoluted mass revealed the formation of a protein fragment 6c with lysine residues and free N-terminus intact (FIG. 4B; FIGS. 44a-44b), as well as the desired bicyclic peptide 6d (FIG. 4C; FIG. 44c). These results indicated the bicyclization occurred exclusively at the cysteine-adjacent lysine residue located at the peptide tail, while the remote lysine residues and N-terminus were unmodified. These experiments demonstrated the crucial role of the cysteine-directed strategy for site-specific lysine bicyclization in complex protein modification.

### Example 11

### Peptide Bicyclization on Bacteriophage

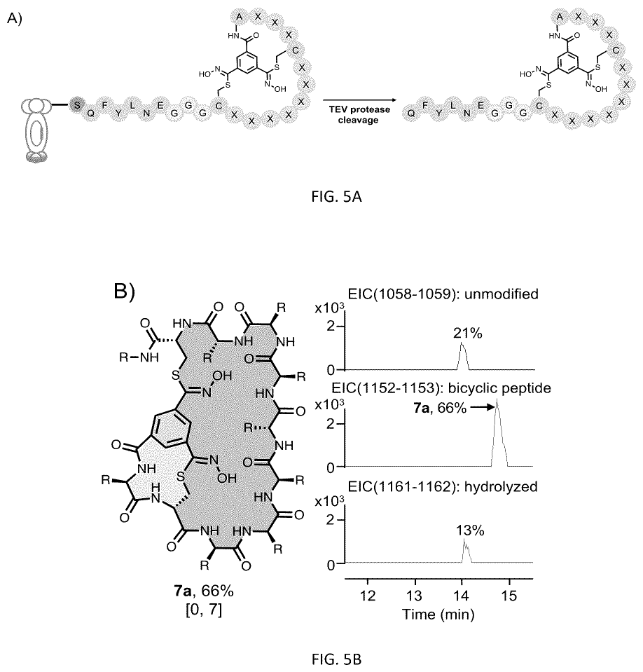

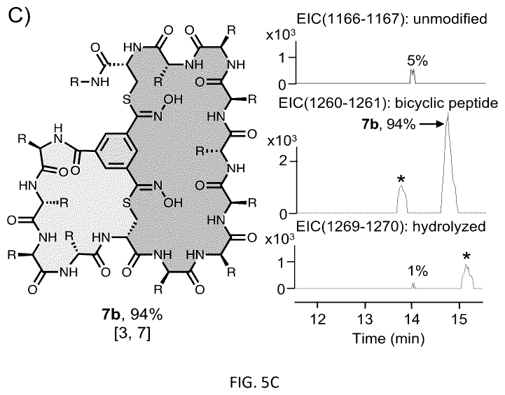

To test the potential of the peptide bicyclization strategy in constructing phage libraries, two M13 bacteriophage variants were constructed that each incorporated a TEV protease cleavage site (ENLYFQ/S (SEQ ID NO: 39)) as well as either an ACX₇C or AX₃CX₇C motif to its N-terminus. Two phage colonies were picked up as representative examples for detailed analysis, which display a peptide sequence of ACSWGIEQRC (SEQ ID NO: 1) (ACX₇C) and AGSACSWGIEQRC (SEQ ID NO: 2) (AX₃CX₇C), respectively. These phage variants were subjected to TCEP reduction and then treated with 20 μM of crosslinker BC-OSu (3b) for bicyclization under mild conditions (2% DMF in PBS, pH 7.4, room temperature) for 20 minutes. Upon completion, the reaction was quenched with 1 mM cysteine for 30 minutes and then the phage was precipitated out of solution using 20% polyethylene glycol-8000 (PEG)/2.5 M NaCl. After treating the modified phage with TEV protease, the modified peptide fragment was separated from the phage particles through centrifugal filtration (FIG. 5A). The modification efficiency was determined by LC-MS analysis using extracted ion chromatogram (EIC) of the unmodified, bicyclic, and hydrolyzed peptides species (FIGS. 49a-49d; FIGS. 50a-50s; FIGS. 51a-51d; FIGS. 52a-52d; FIGS. 53a-53d). As shown in FIG. 5B, the ACX₇C phage construct was modified using the bicyclization protocol with 3b to give 66% of bicyclic peptide, along with 21% of the unmodified peptide and 13% of hydrolytic side-product. Interestingly, when the AX₃CX₇C phage construct with extended spacer was modified with 3b, a high efficiency (94%) of bicyclization was achieved, with only 5% of the sample remained unmodified, 1% of the sample was hydrolyzed (FIG. 5C). These results indicate that the phage-displayed peptide was successfully modified to form a desired bicyclic product through N-terminus-Cys-Cys stapling. These model studies suggest that a bicyclic AX₃CX₇C phage library could be readily constructed using the cysteine-directed proximity-driven bicyclization protocol, providing new structure diversity for peptide therapeutics.

### Example 12

### Bicyclic Phage Screening Against the SARS-CoV-2 Spike RBD Protein

A bicyclic phage screening was performed against the SARS-CoV-2 spike RBD protein. To modify the AX₃CX₇C phage library, the phage were subjected to TCEP reduction (1 mM final concentration in 20 mM NH₄HCO₃ pH 8) for 1 hour and then the phage were precipitated using 20% PEG-800/2.5 M NaCl. The precipitated phage were resuspended in PBS pH 7.4 and then modified with the BC-OSu crosslinker (10 μM final concentration) for 20 minutes and then quenched with cysteine (1 mM final concentration) for 30 minutes. The modified phage were precipitated and then used in the phage screening against the spike protein. For the screening, the biotinylated spike protein (1 ug) was bound to streptavidin magnetic beads (10 μL) for 30 minutes and the unbound protein were washed away. Separately the modified phage library and spike protein on streptavidin beads were blocked with BSA (1 mg/mL final concentration) for 30 minutes to help eliminate non-specific binding. The modified phage were then added to the spike protein and incubated for 30 minutes. The unbound phage were washed away using 0.1% Tween-20 in PBS for 10 washes. The bound phage were eluted by acid release (0.2 M Glycine-HCl, pH 2.2) for 10 minutes and then neutralized (1 M Tris-HCl pH 9.1). The phage output was then amplified in the specific E. coli strain, ER2738 and subsequent rounds of screening were performed using the same protocol. The phage outputs for each round were titered and sequenced to determine if there were any potential peptide hits identified.

Through these screening efforts, multiple peptide hits were identified and characterized using an ELISA. In the ELISA, a 96-well plate was coated with 100 nM of SARS-CoV-2 Spike RBD (100 μL in 100 mM NaHCO₃ pH 8.5) overnight and the following day the wells were washed 3 times with 150 μL of 0.1% Tween in PBS to remove the unbound protein. The wells were blocked with BSA (20 mg/mL in PBST) for 3 hours and then the biotinylated peptide samples at increasing concentrations were added to the wells and incubated for 1 hour. The wells were washed 4 times and then incubated for 1 hour with streptavidin-HRP (100 μL at a 1:100 dilution in blocking buffer). The wells were washed 4 times and then incubated with 50 uL of TMB as the HRP substrate for 15 minutes in the dark. The reaction was quenched using 2 M sulfuric acid and the absorbance at 450 nm was read. The two most potent bicyclic peptide binders identified were, AIDSCRYNHGWQC (SEQ ID NO: 40) (NP8) with a Kd of 3 μM and AISECNHIAEWMC (SEQ ID NO: 41) (NP10) with a Kd of 400 nM.

### REFERENCES

[1] T. Dang, R. D. Sussmuth, Acc. Chem. Res. 2017, 50, 1566-1576.
[2] C. Morrison, Nat. Rev. Drug Discov. 2018, 17, 531-533.
[3] a) C. A. Rhodes, D. Pei, Chem. Eur. J. 2017, 23, 12690-12703; b) V. Baeriswyl, C. Heinis, ChemMedChem 2013, 8, 377-384; c) H. Zhang, S. Chen, RSC Chem. Biol. 2022, 3, 18-31.
[4] I. R. Rebollo, A. Angelini, C. Heinis, MedChemComm 2013, 4, 145-150.
[5] C. Heinis, G. Winter, Curr. Opin. Chem. Biol. 2015, 26, 89-98.
[6] H. Dotter, M. Boll, M. Eder, A.-C. Eder, Biotechnol. Adv. 2021, 47, 107699.
[7] a) L. Reguera, D. G. Rivera, Chem. Rev. 2019, 119, 9836-9860; b) I. V. Smolyar, A. K. Yudin, V. G. Nenajdenko, Chem. Rev. 2019, 119, 10032-10240; c) C. J. White, A. K. Yudin, Nat. Chem. 2011, 3, 509-524; d) J. Ceballos, E. Grinhagena, G. Sangouard, C. Heinis, J. Waser, Angew. Chem. Int. Ed. 2021, 60, 9022-9031.
[8] a) M. Muttenthaler, G. F. King, D. J. Adams, P. F. Alewood, Nat. Rev. Drug Discovery 2021, 20, 309-325; b) C. Bechtler, C. Lamers, RSC Med. Chem. 2021, 12, 1325-1351.
[9] a) C. Heinis, T. Rutherford, S. Freund, G. Winter, Nat. Chem. Biol. 2009, 5, 502-507; b) Schmid et al J. Mol. Biol. 2005, 354, 666-678. c) I. Rentero Rebollo, C. Heinis, Methods 2013, 60, 46-54; d) I. Rentero Rebollo, S. McCallin, D. Bertoldo, J. M. Entenza, P. Moreillon, C. Heinis, ACS Med. Chem. Lett. 2016, 7, 606-611; e) A. Angelini, L. Cendron, S. Chen, J. Touati, G. Winter, G. Zanotti, C. Heinis, ACS Chem. Biol. 2012, 7, 817-821. f) S. Chen, D. Bertoldo, A. Angelini, F. Pojer, C. Heinis, Angew. Chem. Int. Ed. 2014, 53, 1602-1606; g) S. Chen, J. Morales-Sanfrutos, A. Angelini, B. Cutting, C. Heinis, ChemBioChem 2012, 13, 1032-1038.
[10] J. Y. K. Wong, R. Mukherjee, J. Miao, O. Bilyk, V. Triana, M. Miskolzie, A. Henninot, J. J. Dwyer, S. Kharchenko, A. lampolska, D. M. Volochnyuk, Y.-S. Lin, L.-M. Postovit, R. Derda, Chem. Sci. 2021, 12, 9694-9703.
[11] S. Chen, I. Rentero Rebollo, S. A. Buth, J. Morales-Sanfrutos, J. Touati, P. G. Leiman, C. Heinis, J. Am. Chem. Soc. 2013, 135, 6562-6569.
[12] a) Y. Zheng, Z. Li, J. Ren, W. Liu, Y. Wu, Y. Zhao, C. Wu. Chem Sci. 2017, 8, 2547-2552. b) S. Lu, Y. Wu, J. Li, X. Meng, C. Hu, Y. Zhao, C. Wu. J. Am. Chem. Soc. 2020, 142, 16285-16291.
[13] a) H. Dong, J. Li, H. Liu, S. Lu, J. Wu, Y. Zhang, Y. Yin, Y. Zhao, C. Wu, J. Am. Chem. Soc. 2022, 144, 5116-5125; b) Y. Huang, M. M. Wiedmann, H. Suga, Chem. Rev. 2019, 119, 10360-10391.
[14] F.-J. Chen, J. Gao, Chem. Eur. J. 2022, 28, e202201843.
[15] F.-J. Chen, M. Zheng, V. Nobile, J. Gao, Chem. Eur. J. 2022, 28, e202200058.
[16] H. F. Motiwala, Y.-H. Kuo, B. L. Stinger, B. A. Palfey, B. R. Martin, J. Am. Chem. Soc. 2020, 142, 1801-1810.
[17] N. J. Smith, K. Rohlfing, L. A. Sawicki, P. M. Kharkar, S. J. Boyd, A. M. Kloxin, J. M. Fox, Org. Biomol. Chem 2018, 16, 2164-2169.
[18] a) M. Haque, N. Forte, J. R. Baker, Chem. Commun. 2021, 57, 10689-10702; b) K. C. Tang, J. Cao, L. M. Boatner, L. Li, J. Farhi, K. N. Houk, J. Spangle, K. M. Backus, M. Raj, Angew. Chem. Int. Ed. 2022, 61, e202112107.
[19] C. B. Rosen, M. B. Francis, Nat. Chem. Biol. 2017, 13, 697-705.
[20] a) Y. Zhang, Q. Zhang, C. T. T. Wong, X. Li, J. Am. Chem. Soc. 2019, 141, 12274-12279; b) M. Todorovic, K. D. Schwab, J. Zeisler, C. Zhang, F. Benard, D. M. Perrin, Angew. Chem. Int. Ed. 2019, 58, 14120-14124; c) K. Kubota, P. Dai, B. L. Pentelute, S. L. Buchwald, J. Am. Chem. Soc. 2018, 140, 3128-3133.
[21] D. E. Hacker, M. Almohaini, A. Anbazhagan, Z. Ma, M. C. Hartman, in Peptide Libraries: Methods and Protocols (Ed.: R. Derda), Springer New York, New York, NY, 2015, pp. 105-117.
[22] S. Chen, S. Lovell, S. Lee, M. Fellner, P. D. Mace, M. Bogyo, Nat. Biotechnol. 2020, 39, 490-498.
[23] a) R. J. B. Schsfer, M. R. Monaco, M. Li, A. Tirla, P. Rivera-Fuentes, H. Wennemers, J. Am. Chem. Soc. 2019, 141, 18644-18648; b) C. Kesornpun, T. Aree, C. Mahidol, S. Ruchirawat, P. Kittakoop, Angew. Chem. Int. Ed. 2016, 55, 3997-4001.
[24] P. S. R. ANJANEYULU, J. V. STAROS, Int. J. Pept. Protein Res. 1987, 30, 117-124.
[25] a) T. C. Bruice, in Methods Enzymol., Vol. 6, Academic Press, 1963, pp. 606-610; b) F. Schneider, Angew. Chem. Int. Ed. 1978, 17, 583-592.
[26] a) K. Bozovičar, T. Bratkovič, Int J Mol Sci 2021, 22, 1611; b) X. Yang, L. P. Beroske, J. Kemmink, D. T. S. Rijkers, R. M. J. Liskamp, Tetrahedron Lett. 2017, 58, 4542-4546.
[27] C. Yang, J. T. Barbieri, N. M. Dahms, C. Chen, Biochem. 2022, 61, 616-624.
[28] J. C. Carrington, W. G. Dougherty, Proc. Natl. Acad. Sci. USA 1988, 85, 3391-3395.

What is claimed is:

1. A method for constructing a bicyclic peptide, comprising:
a) providing a peptide precursor,
b) labeling cysteine to trigger proximity-driven amine-selective cyclization; and
c) yielding bicyclic peptides with a Cys-Lys-Cys, Lys-Cys-Lys, or N-terminus-Cys-Cys stapling pattern.

2. The method of claim 1, wherein cysteine is conjugated to a panel of multi-functional crosslinkers.

3. The method of claim 2, wherein the multi-functional crosslinker is chlorooxime-based and sets up proximity-driven conjugation with amines to provide bicyclic peptides.

4. The method of claim 3, wherein the multi-functional crosslinker is selected from the group consisting of BC-TFP (3a), BC-Osu(3b), BC-DFP (3c) and CO-Bis-Osu (3h)

5. The method of claim 3, wherein the bicyclic peptide is generated through proximity-driven lysine condensation by Cys labeling, and wherein the crosslinker is installed to the peptide precursor to favor intramolecular amide bond formation.

6. The method of claim 1, wherein the peptide precursor is a natural linear peptide or a protein-fused peptide.

7. A phage library comprising bacteriophage variants comprising bicyclization peptides generated by the method of claim 1.

8. The phage library of claim 7, wherein the bicyclization peptide comprises an amino acid sequence of ACX₇C or AX₃CX₇C, wherein X represents randomized proteinogenic amino acids.

9. The phage library of claim 7, wherein the bacteriophage variants are M13 bacteriophage variants.

10. A method of constructing a phage library comprising bicyclization peptides, said method comprising:
a) constructing a phage library comprising a protease cleavage site and bicyclizable peptide motifs attached to a N-terminus of a phage coat protein;
b) treating the constructed phage library from a) with a multi-functional crosslinker for bicyclization;
c) quenching treated phage variants from b) with cysteine and then selecting the phage variants;
d) treating randomly selected phage variants with protease and centrifugal filtrating the treated phage variants; and
e) analyzing a peptide filtrate to confirm efficient bicyclization of the phage variants.

11. The method of claim 10, wherein the multi-functional crosslinker is chlorooxime-based that is set up proximity-driven conjugation with amines.

12. The method of claim 11, wherein the multi-functional crosslinker is selected from the group consisting of BC-TFP (3a), BC-Osu(3b), BC-DFP (3c) and CO-Bis-Osu (3h).

13. The method of claim 11, wherein the multi-functional crosslinker is BC-Osu(3b).

14. The method of claim 10, wherein the bicyclic peptide motif displays an amino acid sequence of ACX₇C or AX₃CX₇C, wherein X represents randomized proteinogenic amino acids.

15. A method for screening a bicyclic phage of claim 7 for a peptide therapeutics against a protein.

16. The method of claim 15, wherein the protein is SARS-CoV-2 spike RBD protein.

17. The method of claim 16, wherein the peptide therapeutics comprises an amino acid sequence of AIDSCRYNHGWQC (SEQ ID NO: 40, NP8).

18. The method of claim 16, wherein the peptide therapeutics comprises an amino acid sequence of AISECNHIAEWMC (SEQ ID NO: 41, NP10).
